SEQUENCE LISTING (21 sequences)

SEQ ID NO: 1 - protein, 5 aa, Mus musculus
DYAMH

SEQ ID NO: 2 - protein, 17 aa, Mus musculus
LISIYSDHTNYNQKFQG

SEQ ID NO: 3 - protein, 12 aa, Mus musculus
NNYGNYGWYFDV

SEQ ID NO: 4 - protein, 10 aa, Mus musculus
SASSSVSYMH

SEQ ID NO: 5 - protein, 7 aa, Mus musculus
DTSKLAS

SEQ ID NO: 6 - protein, 9 aa, Mus musculus
FQGSGYPYT

SEQ ID NO: 7 - protein, 121 aa, Artificial Sequence
  H2L5 Vh
QVQLVQSGAEVKKPGSSVKVSCKASGYTFTDYAMHWVRQAPGQGLEWMGLISIYSDHTNYNQKFQGRVTITADKSTSTAYMELSSLRSEDTAVYYCGRNNYGNYGWYFDVWGQGTTVTVSS

SEQ ID NO: 8 - protein, 106 aa, Artificial Sequence
  H2L5 Vl
EIVLTQSPATLSLSPGERATLSCSASSSVSYMHWYQQKPGQAPRLLIYDTSKLASGIPARFSGSGSGTDYTLTISSLEPEDFAVYYCFQGSGYPYTFGQGTKLEIK

SEQ ID NO: 9 - protein, 168 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKM

SEQ ID NO: 10 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 11 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 12 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 13 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 14 - protein, 116 aa, Artificial Sequence
  37A10S713 Vh
EVQLVESGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 15 - protein, 111 aa, Artificial Sequence
  37A10S713 Vl
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 16 - protein, 10 aa, Artificial Sequence
  37A10S713 Vh CDR1
GFTFSDYWMD

SEQ ID NO: 17 - protein, 17 aa, Artificial Sequence
  37A10S713 Vh CDR2
NIDEDGSITEYSPFVKG

SEQ ID NO: 18 - protein, 8 aa, Artificial Sequence
  37A10S713 Vh CDR3
WGRFGFDS

SEQ ID NO: 19 - protein, 15 aa, Artificial Sequence
  37A10S713 Vl CDR1
KSSQSLLSGSFNYLT

SEQ ID NO: 20 - protein, 7 aa, Artificial Sequence
  37A10S713 Vl CDR2
YASTRHT

SEQ ID NO: 21 - protein, 9 aa, Artificial Sequence
  37A10S713 Vl CDR3
HHHYNAPPT

(19) United States
(12) Patent Application Publication
(10) Pub. No.: US 2020/0190194 A1
(43) Pub. Date: Jun. 18, 2020

(54) COMBINATION THERAPY

(71) Applicant: GLAXOSMITHKLINE INTELLECTUAL PROPERTY DEVELOPMENT LIMITED, BRENTFORD (GB)

(72) Inventors: Meixia BI, Collegeville, PA (US); Christopher B. HOPSON, Collegeville, PA (US); Patrick A. MAYES, Devon, PA (US); Sapna YADA VILLI, Collegeville, PA (US)

(21) Appl. No.: 16/620,627

(22) PCT Filed: Jun. 8, 2018
(86) PCT No.: PCT/IB2018/054167

§ 371 Date: Dec. 9, 2019

Related U.S. Application Data
(60) Provisional application No. 62/517,309, filed on Jun. 9, 2017.
(60) Provisional application No. 62/662,278, filed on Apr. 25, 2018.

(51) Int. Cl. C07K 16/28; A61P 35/00

(52) U.S. Cl. CPC C07K 16/2818; C07K 16/2827; A61K 2039/507; C07K 2317/75; C07K 2317/76; A61P 35/00

(57) ABSTRACT

The present invention provides methods of treating cancer in a patient in need thereof, the method comprising administering to the patient an effective amount of an agent directed to human ICOS and an effective amount of an agent directed to human PD1 or human PD-L1 sequentially. The present invention also provides an anti-ICOS antibody or antigen binding fragment thereof and an anti-PD1 antibody or antigen binding fragment thereof for sequential use in treating cancer in a human in need thereof. The present invention provides an anti-ICOS antibody or antigen binding fragment thereof and an anti-PD-L1 antibody or antigen binding fragment thereof for sequential use in treating cancer in a human in need thereof.

### FIELD OF THE INVENTION

The present invention relates generally to immunotherapy in the treatment of human disease. More specifically, the present invention relates to the use of sequenced dosing of immunomodulators such as anti-ICOS antibodies, anti-PD1 antibodies, and anti-PDL1 antibodies in the treatment of cancer.

### BACKGROUND OF THE INVENTION

Cancer immunity is a multistep process that is tightly regulated by a series of negative immune checkpoint and positive co-stimulatory receptors that when effectively triggered can achieve antitumor response (Mellman, I., et al. (2011) Cancer Immunotherapy Comes of Age. Nature 480(7378), 480-489). However, tumors have established various mechanisms to circumvent immune clearance by altering the responsiveness of the immune infiltrate. In some instances, tumors will be highly dependent on a single mechanism, and in these cases, there is the potential to achieve significant clinical activity with single agent immunomodulatory therapy (Hoos, A. (2016). Development of immuno-oncology drugs—from CTLA4 to PD1 to the next generations. Nat Rev Drug Discov. 15(4), 235-47). However, as tumors often utilize multiple, overlapping and redundant mechanisms to block antitumor immune response, combination therapy will likely be required for durable efficacy across a wide range of tumor types. Therefore, new immune targeted therapies are needed to improve the treatment of all cancers.

Thus, there is a need for combination treatments and strategies for dosing of immunomodulators for the treatment of disease, in particular cancer.

### BRIEF DESCRIPTION OF THE DRAWINGS

FIG. 1 is a table showing the study design of anti-ICOS antibody/anti-PD1 antibody concurrent and phased dosing study described herein.

FIG. 2 is a schematic showing the study procedure of anti-ICOS antibody/anti-PD1 antibody concurrent and phased dosing study described herein. Shown at the bottom of FIG. 2 is a table listing antibodies used in the study.

FIG. 3 is a plot showing average tumor volume of mice groups treated with anti-ICOS antibody and anti-PD1 antibody concurrently or in sequential phases (e.g., lead-in dose/follow-up dose) and group(s) treated with control(s), as indicated in the figure legend.

FIG. 4 is a plot showing average tumor volume of mice groups treated with concurrent dosing of anti-ICOS antibody and anti-PD1 antibody, and group(s) treated with control(s), as indicated in the figure legend.

FIG. 5 is a plot showing average tumor volume of mice groups treated with phased dosing of anti-ICOS antibody and anti-PD1 antibody, and group(s) treated with control(s), as indicated in the figure legend.

FIG. 6 is a plot showing average tumor volume of mice groups treated with phased dosing of anti-PD1 antibody and anti-ICOS antibody, with anti-PD1 antibody as lead-in dose and anti-ICOS antibody as follow-up dose, and group(s) treated with control(s), as indicated in the figure legend.

FIG. 7 is a plot showing average tumor volume of mice groups treated with phased dosing of anti-ICOS antibody and anti-PD1 antibody, with anti-ICOS antibody as lead-in dose and anti-PD 1 antibody as follow-up dose, and group(s) treated with control(s), as indicated in the figure legend.

FIGS. 8A-8C are sets of plots showing individual tumor volumes of mice treated with concurrent dosing of anti-ICOS antibody and anti-PD1 antibody, and group(s) treated with control(s), as indicated in the corresponding figure legend(s). FIG. 8A shows individual tumor volumes of mice in Group 1 (left) and Group 2 (right). FIG. 8B shows individual tumor volumes of mice in Group 3 (top left), Group 4 (top right), and Group 5 (bottom). FIG. 8C shows individual tumor volumes of mice in Group 6 (left) and Group 7 (right).

FIGS. 9A-9C are sets of plots showing individual tumor volumes of mice treated with phased dosing of anti-ICOS antibody and anti-PD1 antibody, and group(s) treated with control(s), as indicated in the corresponding figure legend(s). FIG. 9A shows individual tumor volumes of mice in Group 1 (left) and Group 2 (right). FIG. 9B shows individual tumor volumes of mice in Group 8 (top left), Group 9 (top right), and Group 10 (bottom).

FIG. 9C shows individual tumor volumes of mice in Group 11 (top left), Group 12 (top right), and Group 13 (bottom).

FIG. 10 is a plot showing survival of mice in all groups (Groups 1-13). Mice in the groups were treated with concurrent or phased dosing of anti-ICOS antibody and anti-PD1 antibody or treated with control(s), as indicated in the figure legend.

FIG. 11 is a plot showing survival of mice groups treated with concurrent dosing of anti-ICOS antibody and anti-PD1 antibody, and group(s) treated with control(s), as indicated in the figure legend.

FIG. 12 is a plot showing survival of mice groups treated with phased dosing of anti-ICOS antibody and anti-PD1 antibody, and group(s) treated with control(s), as indicated in the figure legend.

FIG. 13 is a plot showing survival of mice groups treated with phased dosing of anti-PD1 antibody and anti-ICOS antibody, with anti-PD1 antibody as lead-in dose and anti-ICOS antibody as follow-up dose, and group(s) treated with control(s), as indicated in the figure legend.

FIG. 14 is a plot showing survival of mice groups treated with phased dosing of anti-ICOS antibody and anti-PD1 antibody, with anti-ICOS antibody as lead-in dose and anti-PD1 antibody as follow-up dose, and group(s) treated with control(s), as indicated in the figure legend.

FIG. 15: Development of an anti-human ICOS agonist monoclonal antibody

(A) H2L5 binding to dimeric human ICOS (B) human ICOS-L binding to dimeric human ICOS (C) Binding of H2L5 (20 μg/mL) to CD4 (**P=0.0011, t=4.183, df=13) and CD8 (**P=0.0078, t=3.686, df=7) T cells from healthy donors. Each symbol represents a separate human donor, horizontal lines indicate median, and bars are interquartile range (D) Representative Western Blot demonstrating induction of AKT signaling in purified activated T cells after treatment with H2L5 (E) Quantification of CD69+CD4 (*P=0.0142, t=3.416 df=6) or CD8 (**P=0.0012, t=5.734 df=6) T cells and (F) quantification of Ki67⁺ CD4 (*P=0.0190, t=3.809 df=4) or CD8 (*P=0.0255, t=3.474 df=4) T cells from healthy donor PBMC treated with (12.5 μg/mL) of bound H2L5 and anti-CD3 for 48 hours. (G, H) Quantification of soluble IFN-γ from (G) the culture supernatant of PBMC from healthy subjects treated with (12.5 μg/mL) of bound H2L5 and anti-CD3 for 24 hours **P=0.0041, t=4.510 df=6 or 48 hrs *P=0.0375, t=2.661 df=6 (H) the supernatant of NSCLC cancer patient PBMC treated with (10 μg/mL) bound H2L5 and anti-CD3 for 72 hours. (I, K) Quantification of RNA expression of (I) T-Bet (TBX21) (*P=0.0156, t=2.974 df=9) and (J) Granzyme B (GZMB) (**P=0.0020, t=4.292 df=9) (K) L-Selectin (SELL) (*P=0.0161, t=2.955 df=9) from healthy donor CD3⁺ T cells following indicated treatments analyzed by a two-tailed, unpaired t-test. H2L5 induces concentration dependent increases in cytokine production and T-cell activation from disaggregated tumor cell suspensions. Plates were coated with H2L5+/−anti-CD3 or isotype control. (L) IFN-γ, (M) CD8+ OX40+, (N) CD8+ CD25+ following 6 days of culture. Bars=Group Medians p<0.05 by One Way Anova, **P<0.05 ***P<0.0005, ****P<0.000 by One Way Anova. Dashed line=CD3+isotype IgG4 10 μg/mL. See FIG. S7 for tumor types.

FIG. 16: Antibody isotype and FcγR-engagement is critical for H2L5 function

(A) PBMC from healthy subjects treated with soluble H2L5 of varying isotypes at 5 μg/ml for 6 days. Proliferation as measured by CFSE dilution relative to isotype control (fold change) (B, C) PBMCs from healthy subjects, with or without depletion of NK cells; treated with (B) soluble H2L5 of varying isotypes at (5 μg/mL) for 6 days. (C) Soluble H2L5 of varying isotypes (10 μg/mL) for 24 hours and percentage of dead cells determined by flow cytometry using NIR Live/Dead dye. An anti-CD52 antibody known to induce ADCC-mediated T-cell killing was included as a positive control. (D) ICOS Expression on freshly dissociated patient TIL. The median fluorescent intensity of ICOS from CD4, CD8, T_{reg}, and T_{eff} cell populations. (Tumor types Solid Triangle=NSCLC (6) Solid Circle=CRC (4) Solid Diamond=Bladder (2) Solid Square=Head/Neck (1) Open Triangle=RCC (4) Open Circle=Endometrial (2) Open Diamond=Prostate (1) Open Square=Thyroid (1); p<0.05 by One Way Anova). Insert shows histogram of ICOS expression on CD4 (Red), CD8 (orange) and T_{reg} (Blue) from a patient with endometrial cancer.
(E) Spearman correlation between total ICOS receptor numbers (calculated by multiplying the percent ICOS positive for each cell type by the ICOS receptor number per positive cell) and FcγRIIIA reporter assay fold induction in target cells isolated from PBMCs and patient tumors in presence of H2L5 IgG1 isotype relative to isotype control for all samples where both data points were available (r²=0.681, p<0.001). (F) Fold induction observed in an FcγRIIIA reporter assay using target cells isolated from NSCLC patient tumor 5001003 incubated with anti-ICOS antibodies. CD4 T_{eff}, CD8 T cells and T_{reg} were isolated from a dissociated patient tumor and utilized as target cells in the FcγRIIIA assay.

FIG. 17: H2L5 exhibits FcR dependent agonism to induce T-cell activation

(A) Isolated CD4 T cells from healthy subjects treated with indicated concentrations of H2L5 for 60 hours (bound isotype vs. bound H2L5 ***P=0.0006, t=9.777 df=4, soluble isotype vs. soluble H2L5 ***P=0.0003, t=11.50 df=4 and (#) bound H2L5 vs. soluble H2L5 **P=0.0017, t=7.530 df=4) (B) PBMC from a healthy subject treated with soluble H2L5 (ICOS IgG4PE) or H2L5 Fc-disabled at (10 μg/mL) for 3.5 days (isotype control vs. H2L5 **P=0.0056, t=5.426 df=4), (H2L5 vs. H2L5 Fc-disabled **P=0.0012, t=8.297 df=4) (C) MLR with anti-CD3 antibody followed by treatment with soluble H2L5 or H2L5 Fc-disabled antibody at (10 μg/mL) (isotype control vs. H2L5 *P=0.0166, t=3.966 df=4), (H2L5 vs. H2L5 Fc-disabled *P=0.0158, t=4.022 df=4) (D) Isolated T cells cultured with and without monocytes from the same donor followed by treatment with soluble H2L5 or H2L5 Fc-disabled at (10 μg/mL)+/−anti-CD32 or Fc-blocking antibody for 4 days. (#) ***P=0.0009, t=8.734 df=4, ($) **P=0.0031, t=6.405 df=4, (&) *P=0.0389, t=3.026 df=4, (@) isotype control vs. H2L5 **P=0.0027, t=6.612 df=4, H2L5 vs. H2L5 Fc-disabled *P=0.0239, t=3.544 df=4, H2L5 (control) vs. H2L5 (anti-CD32) **P=0.0066, t=5.184 df=4, H2L5 (anti-CD32) vs. H2L5 (Fc block) **P=0.0013, t=8.047 df=4 and H2L5 (control) vs. H2L5 (Fc block) *P=0.0446, t=2.889 df=4. (E, F) Human T cells pre-stimulated with anti-CD3 for 48 hours and added to a co-culture with human DC. AlexaFlour488-labeled H2L5 IgG4PE added at 3 μg/mL to co-cultures on ice then moved to 37° C. for indicated timepoints. Arrows indicate T cells activated in response to H2L5 treatment, polarization and mobilization towards neighboring dendritic cell. Data representative of three separate experiments performed using different donor cells.

FIG. 18: H2L5 induces an EM phenotype and anti-tumor activity in humanized mouse model.

(A) Quantification of human CD45⁺CD3⁺ cells in the blood of mice H2L5 treatments as compared to isotype control IgG4PE (****P=<0.0001, F=33.57, df=24) (B) Quantification of human CD45⁺CD3⁺CD69⁺ cells from the blood of mice H2L5 (1.2 mg/kg) vs. isotype control IgG4PE (*P=0.0119, F=4.179, df=24) (C) Percentage of CD4 T_{CM} (0.04 mg/kg **P=0.0038, 0.4 mg/kg ***P=0.0002, 1.2 mg/kg ***P=0.0005, F=8.172, df=20. This is equivalent to 0.8, 8 and 24 μg per mouse respectively. (D) CD8⁺ Tₙₐᵢᵥₑ/terminally differentiated effector memory T_{TEMRA} (0.004 mg/kg **P=0.0036, 0.04 mg/kg and 0.4 mg/kg ****P=<0.0001, 1.2 mg/kg **P=0.0072, F=13.78, df=20) in the spleen of mice (E) The percentage of ICOS+ or PD-1+ T cells in mice implanted subcutaneously with A549 tumor and identified by using PE conjugated mouse anti human IgG4 by flow cytometry. (F) The ratio of CD8/T_{reg} cells in whole tumor tissues (G) HCT116 tumor volumes on day 13 (0.04 mg/kg) *P=0.0273, (0.4 mg/kg) *P=0.0432, F=2.788, df=36 (H) A549 tumor volumes on day 21 (0.4 mg/kg) *P=0.0056, F=3.906, df=36 (i) Kaplan-Meier survival curve of human PBMC engrafted NSG mice with A549 tumors (A-I) horizontal lines represent median values, error bars represent interquartile range. All statistical tests were one-way ANOVA.

FIG. 19: The isotype of the murine ICOS mAb influences efficacy in syngeneic tumors.

(A) Kaplan-Meier plots of mice with murine (A) EMT6 murine (B) CT26 syngeneic tumors treated with indicated doses (5, 100 or 200 μg corresponding to 0.5, 5 and 10 mg/Kg respectively of murine IgG1 or IgG2a versions of 7E.17G9 antibody twice weekly for 3 weeks or isotype control (200 μg or 10 mg/kg). Results are representative of two repeat experiments. Each symbol represents an individual mouse. Horizontal lines represent median values, error bars represent interquartile range. All statistical tests were one-way ANOVA, followed by specific treatment comparators. (C) The ratio of CD8⁺/T_{reg} in EMT6 or CT26 tumors determined at tumor size 100 mm³; (D) The percentage of ICOS+ CD4, CD8 and T_{reg} cells in tumors (closed circles) or spleens (open circles) of mice implanted with EMT6 tumors; MFI of ICOS on CD8, CD4 and T_{reg} in tumors (closed circles) or spleen (open circles) in mice implanted with (E) CT26 or (F) EMT6 tumors at tumor sizes of 100 mm³. (G) Histogram of representative flow plot comparing MFI of ICOS expression on CD4, CD8 and T_{reg} isolated from EMT6 and CT26 tumors; (H) Absolute number of TCR clones expanded in post-treatment with anti-ICOS 7E.17G9 blood that were also found in EMT6 tumor (10 □g *P=0.0173 and 100 g *P=0.0483; F=3.269 df=28).

FIG. 20: Evaluation of ICOS expression on different cell types in human cancers. (A) Expression of ICOS, ICOS-L and PD-L1 in different tumor types ranked by expression of ICOS from TCGA database. (B) The expression of ICOS+ cells by single plex IHC and correlation with expression of PD-L1, PD-1, CD4, CD8, FOXP3 and CD3 in NSCLC. (C) % CD45+ cells that are CD3+, B cells, monocytes, NK cells, macrophages, dendritic cells in disaggregated tumors from different solid tumor types. Solid Triangle=NSCLC (6) Solid Circle=CRC (4) Solid Diamond=Bladder (2) Solid Square=Head/Neck (1) Open Triangle=RCC (4) Open Circle=Endometrial (2) Open Diamond=Prostate (1) Open Square=Thyroid (1). (D) The percentage CD3+CD8+, CD3+CD4+Foxp3+ (T_{reg}) and the ratio of CD3+, CD8+: CD3+CD4+Foxp3 in different tumor types. Horizontal line shows median. (E) Quantification of the co-expression of CD3+PD-1+ICOS+ cells in tumor biopsies obtained from different tumour types by multiplex IHC. (F) Multiplex IHC of a Head and neck FFPE tumor sample co-stained for CD3, PD-1 and ICOS (G) Heatmap summarizing the differentially expressed genes in purified human T cells treated with H2L5 plus anti-CD3 mAb compared to anti-CD3 alone as determined by NanoString nCounter analysis System using Human PanCancer-Immune profiling panel (N=6 donors). (H) Gene expression changes (fold increase) common between anti-CD3 (0.6 μg/mL) plus H2L5 (10 μg/mL) activated human T cells (n=6 donors) and murine EMT6 transplantable tumors after surrogate anti-ICOS (7E.17G9 rat IgG2b) treatment.

FIG. 21: ICOS agonist mAb induce PD-1/PD-L1 expression and enhances activity of anti-PD-1

(A) Quantification of RNA expression of PD-L1 (CD274) (10 μg *P=0.0137 and 100 μg *P=0.0374; F=5.175 df=10) and (B) PD-1 (Pdcd1) (10 μg *P=0.0194 and 100 μg P=0.1626; F=3.911 df=10) in EMT6 following indicated treatments. Each symbol represents an individual mouse sample, horizontal lines represent median values, error bars represent interquartile range. All statistical tests were one-way ANOVA with square root transformed data to stabilize variances (C) Percentage of CD4⁺PD-1⁺ and CD8⁺PD-1⁺ T cells following treatment with isotype control or H2L5 at 10 μg/mL for 72 hours in PBMC from cancer patients CD4⁺ *P=0.0128, t=3.026 df=10; CD8⁺ **P=0.005, t=3.548, df=10. two-tailed, unpaired t-tests (D). Percentage of CD4+ ICOS+ in NSCLC or melanoma patients pre- and post-PD-1 therapy (either pembrolizumab or nivolumab) compared with healthy subjects. (E) Mice with EMT6 tumors treated with 7E.17G9 IgG1 (10 μg equivalent to 0.5 mg/kg), anti-PD-1 (200 μg equivalent to 10 mg/kg) or the combination of 7E.17G9 and anti-PD-1 dosed concomitantly, twice weekly for 3 weeks. (N=10 per treatment group) (F) A549 tumor volume in NSG mice reconstituted with human PBMC and treated with H2L5 at 0.8 μg mouse equivalent to 0.04 mg/kg, isotype 0.8 μg equivalent to 0.04 mg/kg or anti-PD-1 (pembrolizumab/Keytruda) 100 μg equivalent to 5 mg/kg or the combination of both antibodies. (G) Quantification of IFN-γ from disseminated NSCLC patient tumors treated with anti-CD3 and H2L5 (10 μg/mL) for 24 hours. (#) **P=0.0100 ($) ****P=<0.0001 (&) ***P=0.002, F=15.8, df=20. Horizontal lines represent median values, error bars represent interquartile range (H) MLR assay evaluating ICOS+pembrolizumab vs. ICOS **P=0.0036, IgG4PE ICOS+pembrolizumab vs. pembrolizumab **P=0.0090, ICOS+pembrolizumab vs. 2× IgG4PE ***P=0.0009, F=7.324, df=10. Bars represent mean of triplicate measurement and error bars represent standard deviation (C-E) All statistical tests were one-way ANOVA)

FIG. 22: H2L5 IgG4PE epitope binding (A) An ICOS-L competition assay by MSD demonstrates that H2L5 IgG4PE partially competes with ICOS-L for binding to human ICOS receptor. (B) Activated T cells were incubated with different concentrations of recombinant ICOS-L (R&D systems) and then incubated with H2L5 and MFI of ICOS CD4+ and CD8+ cells determined by flow cytometry.

FIG. 23: H2L5IgG4PE causes dose dependent increases in (A) cytokine production IFNγ, IL-17, IL-10, IL-4, IL-13, IL-5, IL-2, IL-6, TNFα measured by MSD (B) activation marker OX40, CD25 and CD69 on CD4 and CD8 T cells. PBMC were cultured for 48 h with anti-CD3 (0.6 ug/ml) and different concentrations of H2L5IgG4PE or isotype control and supernatants harvested for cytokine analysis and cells for flow cytometry.

FIG. 24: H2L5 induces concentration dependent increases in cytokine production from disaggregated tumor cell suspensions from different cancer patients. Disaggregated tumor cells suspensions were cultured with plate bound H2L5IgG4PE or isotype control in the presence or absence of anti-CD3 following 6 day in vitro stimulation with plate bound anti-CD3 (0.6 μg/mL) and IL2 (100 ng/mL) followed by analysis of (A) IL17, (B) IL10, (C) IL5, (D) IL13 cytokines in the supernatants by MSD.

FIG. 25: H2L5 induces concentration dependent increases on percentage of (A) CD8+LAG3+, p<0.005 by One Way Anova (B) CD8+ PD-1+, (C) ICOS L+cells and (D) (CD4+, CD25+ Foxp3+) p<0.05 by One Way Anova from disaggregated tumor cell suspensions from different cancer patients. Disaggregated tumor cells suspensions were cultured with plate bound H2L5 (ICOS) IgG4PE or isotype control in the presence or absence of anti-CD3 following 6 day in vitro stimulation with plate bound anti-CD3 (0.6 μg/mL) and IL-2(100 ng/mL) followed by flow cytometry. Dashed line=CD3+IgG4 isotype 10 μg/mL Horizontal bars represent median.

FIG. 26: H2L5 IgG1 induces signaling via the major activating FcγR (FcγRIIIa) responsible for ADCC in humans. (A) Treatment of Jurkat-FcγRIIIA-NFAT-luciferase effector cells and primary human CD4⁺ T cells at a ratio of 6:1 with soluble H2L5 of varying isotypes for 6 hrs. An anti-CD52 antibody known to induce ADCC-mediated T cell killing was included as a positive control (B) Treatment of Jurkat-FcγRIIIA-NFAT-luciferase effector cells and purified primary human ex vivo tumor derived CD4, CD8 and Tregs at a ratio of 6:1 with soluble H2L5 IgG1 for 6 hrs Fold change in luciferase induction produced by Jurkat-FcγRIIIA-NFAT-luciferase effector cells relative to isotype control.

FIG. 27: H2L5 causes dose dependent binding to ICOS expressing T cells in blood and tumor. The percentage of ICOS+ or PD-1+ T cells in whole blood (A) and tumor tissues (B) within each group, 48 hours post 4^{th} dose identified using PE conjugated mouse anti human IgG4 by flow cytometry. Bars represent the median values for each group.

FIG. 28: Characterization of an anti-murine ICOS agonist antibody. Anti-mouse ICOS agonist antibody (7E.17G9) induces IFNγ production in disseminated mouse splenocytes cultured ex vivo for 60 hours.

FIG. 29: Tumor growth for (A) EMT6 or (B) CT26 murine syngeneic tumors treated with 10 (0.5 mg/kg), 100 (5 mg/kg) or 200 μg (10 mg/kg) doses of murine IgG1 or Ig2a variants of 7E.17G9 antibody or isotype control (200 g (10 mg/kg) twice weekly for 3 weeks. *(numbers) indicate the number of mice with minimally detectable or non-detectable tumors at study endpoint.

FIG. 30: % ICOS+ cells within CD4, CD8 and T_{reg} populations in tumors (closed circles) and spleens (open circles) of mice bearing ˜100 mm3 CT26 tumors.

FIG. 31: (A) absolute number of TCR clones contracted in post-treatment with Anti-ICOS 7E17G9 antibody blood relative to pre-treatment blood (10 μg *P=0.0327 and 100 μg *P=0.0497; F=3.033 df=28) (B) absolute number of TCR clones expanded in post-treatment blood relative to pre-treatment blood (10 μg P=0.0975 and 100 μg P=0.1915; F=1.958 df=28) (C) Mean T cell fraction estimate vs Mean productive clonality

FIG. 32: Expression of ICOS positive cells in NSCLC, Breast cancer and CRC by IHC singleplex Immunohistochemical detection of ICOS in non-small cell lung cancer (NSCLC), breast cancer (BrCA) TNBrCa, and colorectal cancer (CRC), using a rabbit anti-human CD278 Monoclonal antibody clone SP98 (Spring Biosciences). Assay was carried out on the Leica Bond RX with associated platform reagents. DAB (3, 3′-diaminobenzidine) was used for target detection. Sections were counter stained with Hematoxylin (All scale bars=20 um).

FIG. 33: Changes on cytokine levels from healthy human donor PBMC in response to treatment with anti-CD3 plus isotype control or H2L5 IgG4PE antibody at 12.5 μg/mL

FIG. 34: Cytokine induction of PBMC from NSCLC patients following treatment with isotype control or H2L5 IgG4PE antibody at 10 μg/mL for 72 hrs.

FIG. 35: Binding affinity of different isotype variants of humanized H2L5 antibody to human FcgR.

FIG. 36: Binding affinity of different isotype variants 7E-17G9 to murine FcR

FIG. 37: mRNA Expression of ICOS positive cells in different tumor pathologies from TCGA

FIG. 38: Gene expression changes with anti CD3+H2L5 treatment compared to CD3 alone in human T cells as measured by Nanostring

### SUMMARY OF THE INVENTION

In one aspect, the present invention provides methods of treating cancer in a patient in need thereof comprising administering to the patient an effective amount of an agent directed to human ICOS and an effective amount of an agent directed to human PD1 or human PD-L1 sequentially, wherein administration of the agent directed to human ICOS is followed by administration of the agent directed to human PD1 or human PD-L1. In one embodiment, the agent directed to human ICOS is an ICOS agonist. In one embodiment, the agent directed to human PD1 or human PD-L1 is a PD1 antagonist.

In one aspect, the present invention provides an anti-ICOS antibody or antigen binding fragment thereof and an anti-PD1 antibody or antigen binding fragment thereof for sequential use in treating cancer in a human in need thereof, wherein administration of the anti-ICOS antibody or antigen binding fragment thereof is followed by administration of the anti-PD1 antibody or antigen binding fragment thereof. In one embodiment, the anti-PD1 antibody or antigen binding fragment thereof is a PD1 antagonist. In one embodiment, the anti-ICOS antibody or antigen binding fragment thereof is an ICOS agonist.

In one aspect, the present invention provides an anti-ICOS antibody or antigen binding fragment thereof and an anti-PD-L1 antibody or antigen binding fragment thereof for sequential use in treating cancer in a human in need thereof, wherein administration of the anti-ICOS antibody or antigen binding fragment thereof is followed administration of the anti-PD-L1 antibody or antigen binding fragment thereof. In one embodiment, the anti-PDL1 antibody or antigen binding fragment thereof is a PD1 antagonist. In one embodiment, the anti-ICOS antibody or antigen binding fragment thereof is an ICOS agonist.

### DETAILED DESCRIPTION OF THE INVENTION

### Definitions

As used herein “ICOS” means any Inducible T-cell costimulator protein. Pseudonyms for ICOS (Inducible T-cell COStimulator) include AILIM; CD278; CVID1, JTT-1 or JTT-2, MGC39850, or 8F4. ICOS is a CD28-superfamily costimulatory molecule that is expressed on activated T cells. The protein encoded by this gene belongs to the CD28 and CTLA-4 cell-surface receptor family. It forms homodimers and plays an important role in cell-cell signaling, immune responses, and regulation of cell proliferation. The amino acid sequence of human ICOS (isoform 2) (Accession No.: UniProtKB-Q9Y6W8-2) is shown below as SEQ ID NO:9.

(SEQ ID NO: 9)
MKSGLWYFFLFCLRIKVLTGEINGSANYEMFIFHNGGVQILCKYPDIV
QQFKMQLLKGGQILCDLTKTKGSGNTVSIKSLKFCHSQLSNNSVSFFL
YNLDHSHANYYFCNLSIFDPPPFKVTLTGGYLHIYESQLCCQLKFWLP
IGCAAFVVVCILGCILICWLTKKM

The amino acid sequence of human ICOS (isoform 1) (Accession No.: UniProtKB-Q9Y6W8-1) is shown below as SEQ ID NO: 10.

| (SEQ ID NO: 10) | | | | |
|---|---|---|---|---|
| MKSGLWYFFL | FCLRIKVLTG | EINGSANYEM | FIFHNGGVQI | LCKYPDIVQQ |
| FKMQLLKGGQ | ILCDLTKTKG | SGNTVSIKSL | KFCHSQLSNN | SVSFFLYNLD |
| HSHANYYFCN | LSIFDPPPFK | VTLTGGYLHI | YESQLCCQLK | FWLPIGCAAF |
| VVVCILGCIL | ICWLTKKKYS | SSVHDPNGEY | MFMRAVNTAK | KSRLTDVTL |

Activation of ICOS occurs through binding by ICOS-L (B7RP-1/B7-H2). Neither B7-1 nor B7-2 (ligands for CD28 and CTLA4) bind or activate ICOS. However, ICOS-L has been shown to bind weakly to both CD28 and CTLA-4 (Yao S et al., “B7-H2 is a costimulatory ligand for CD28 in human”, Immunity, 34(5); 729-40 (2011)). Expression of ICOS appears to be restricted to T cells. ICOS expression levels vary between different T cell subsets and on T cell activation status. ICOS expression has been shown on resting TH17, T follicular helper (TFH) and regulatory T (Treg) cells; however, unlike CD28; it is not highly expressed on naïve T_{H}1 and T_{H}2 effector T cell populations (Paulos C M et al., “The inducible costimulator (ICOS) is critical for the development of human Th17 cells”, Sci Transl Med, 2(55); 55ra78 (2010)). ICOS expression is highly induced on CD4+ and CD8+ effector T cells following activation through TCR engagement (Wakamatsu E, et al., “Convergent and divergent effects of costimulatory molecules in conventional and regulatory CD4+ T cells”, Proc Natal Acad Sci USA, 110(3); 1023-8 (2013)). Co-stimulatory signalling through ICOS receptor only occurs in T cells receiving a concurrent TCR activation signal (Sharpe A H and Freeman G J. “The B7-CD28 Superfamily”, Nat. Rev Immunol, 2(2); 116-26 (2002)). In activated antigen specific T cells, ICOS regulates the production of both T_{H}1 and T_{H}2 cytokines including IFN-γ, TNF-α, IL-10, IL-4, IL-13 and others. ICOS also stimulates effector T cell proliferation, albeit to a lesser extent than CD28 (Sharpe A H and Freeman G J. “The B7-CD28 Superfamily”, Nat. Rev Immunol, 2(2); 116-26 (2002)). Antibodies to ICOS and methods of using in the treatment of disease are described, for instance, in WO2012/131004, US20110243929, and US20160215059. US20160215059 is incorporated by reference herein. CDRs for murine antibodies to human ICOS having agonist activity are shown in PCT/EP2012/055735 (WO 2012/131004). Antibodies to ICOS are also disclosed in WO 2008/137915, WO 2010/056804, EP 1374902, EP1374901, and EP1125585. Agonist antibodies to ICOS or ICOS binding proteins are disclosed in WO2012/13004, WO2014/033327, WO2016/120789, US20160215059, and US20160304610. Exemplary antibodies in US2016/0304610 include 37A10S713. Sequences of 37A10S713 are reproduced below as SEQ ID NOS: 14-21.

| 37A10S713 heavy chain variable region: | | | | |
|---|---|---|---|---|
| (SEQ. ID NO : 14) | | | | |
| EVQLVESGG | LVQPGGSLRL | SCAASGFTFS | DYWMDWVRQA | PGKGLVWVSN |
| IDEDGSITEY | SPFVKGRFTI | SRDNAKNTLY | LQMNSLRAED | TAVYYCTRWG |
| RFGFDSWGQG | TLVTVSS | | | |
| 37A10S713 light chain variable region: | | | | |
| (SEQ. ID NO: 15) | | | | |
| DIVMTQSPDS | LAVSLGERAT | INCKSSQSLL | SGSFNYLTWY | QQKPGQPPKL |
| LIFYASTRHT | GVPDRFSGSG | SGTDFTLTIS | SLQAEDVAVY | YCHHHYNAPP |
| TFGPGTKVDI | K | | | |

37A10S713 V_{H} CDR1:
(SEQ. ID NO: 16)
GFTFSDYWMD
37A10S713 V_{H} CDR2:
(SEQ. ID NO: 17)
NIDEDGSITEYSPFVKG
37A10S713 V_{H} CDR3:
(SEQ. ID. NO: 18)
WGRFGFDS
37A10S713 V_{L} CDR1:
(SEQ. ID NO: 19)
KSSQSLLSGSFNYLT
37A10S713 V_{L} CDR2:
(SEQ. ID NO: 20)
YASTRHT
37A10S713 V_{L} CDR3:
(SEQ. ID NO: 21)
HHHYNAPPT

By “agent directed to ICOS” is meant any chemical compound or biological molecule capable of binding to ICOS. In some embodiments, the agent directed to ICOS is an ICOS binding protein. In some other embodiments, the agent directed to ICOS is an ICOS agonist.

The term “ICOS binding protein” as used herein refers to antibodies and other protein constructs, such as domains, which are capable of binding to ICOS. In some instances, the ICOS is human ICOS. The term “ICOS binding protein” can be used interchangeably with “ICOS antigen binding protein.” Thus, as is understood in the art, anti-ICOS antibodies and/or ICOS antigen binding proteins would be considered ICOS binding proteins. As used herein, “antigen binding protein” is any protein, including but not limited to antibodies, domains and other constructs described herein, that binds to an antigen, such as ICOS. As used herein “antigen binding portion” of an ICOS binding protein would include any portion of the ICOS binding protein capable of binding to ICOS, including but not limited to, an antigen binding antibody fragment.

In one embodiment, the ICOS antibodies of the present invention comprise any one or a combination of the following CDRs:

CDRH1:
(SEQ ID NO: 1)
DYAMH
CDRH2:
(SEQ ID NO: 2)
LISIYSDHTNYNQKFQG
CDRH3:
(SEQ ID NO: 3)
NNYGNYGWYFDV
CDRL1:
(SEQ ID NO: 4)
SASSSVSYMH
CDRL2:
(SEQ ID NO: 5)
DTSKLAS
CDRL3:
(SEQ ID NO: 6)
FQGSGYPYT

In some embodiments, the anti-ICOS antibodies of the present invention comprise a heavy chain variable region having at least 90% sequence identity to SEQ ID NO:7. Suitably, the ICOS binding proteins of the present invention may comprise a heavy chain variable region having about 85%, 86%, 87%, 88%, 89%, 90%, 91%, 92%, 93%, 94%, 95%, 96%, 97%, 98%, 99%, or 100% sequence identity to SEQ ID NO:7.

| Humanized Heavy Chain (Vu) Variable Region (H2): | | | |
|---|---|---|---|
| (SEQ ID NO: 7) | | | |
| QVQLVQSGAE VKKPGSSVKV | SCKASGYTFT | DYAMHWVRQA | PGQGLEWMGL |
| ISIYSDHTNY NQKFQGRVTI | TADKSTSTAY | MELSSLRSED | TAVYYCGRNN |
| YGNYGWYFDV WGQGTTVTVS | S | | |

In one embodiment of the present invention the ICOS antibody comprises CDRL1 (SEQ ID NO:4), CDRL2 (SEQ ID NO:5), and CDRL3 (SEQ ID NO:6) in the light chain variable region having the amino acid sequence set forth in SEQ ID NO:8. ICOS binding proteins of the present invention comprising the humanized light chain variable region set forth in SEQ ID NO:8 are designated as “L5.” Thus, an ICOS binding protein of the present invention comprising the heavy chain variable region of SEQ ID NO:7 and the light chain variable region of SEQ ID NO:8 can be designated as H2L5 herein.

In some embodiments, the ICOS binding proteins of the present invention comprise a light chain variable region having at least 90% sequence identity to the amino acid sequence set forth in SEQ ID NO:8. Suitably, the ICOS binding proteins of the present invention may comprise a light chain variable region having about 85%, 86%, 87%, 88%, 89%, 90%, 91%, 92%, 93%, 94%, 95%, 96%, 97%, 98%, 99%, or 100% sequence identity to SEQ ID NO:8.

| Humanized Light Chain (VI) Variable Region (L5) | | | | |
|---|---|---|---|---|
| (SEQ ID NO: 8) | | | | |
| EIVLTQSPAT | LSLSPGERAT | LSCSASSSVS | YMHWYQQKPG | QAPRLLIYDT |
| SKLASGIPAR | FSGSGSGTDY | TLTISSLEPE | DFAVYYCFQG | SGYPYTFGQG |
| TKLEIK | | | | |

CDRs or minimum binding units may be modified by at least one amino acid substitution, deletion or addition, wherein the variant antigen binding protein substantially retains the biological characteristics of the unmodified protein, such as an antibody comprising SEQ ID NO:7 and SEQ ID NO:8.

It will be appreciated that each of CDR H1, H2, H3, L1, L2, L3 may be modified alone or in combination with any other CDR, in any permutation or combination. In one embodiment, a CDR is modified by the substitution, deletion or addition of up to 3 amino acids, for example 1 or 2 amino acids, for example 1 amino acid. Typically, the modification is a substitution, particularly a conservative substitution, for example as shown in Table 1 below.

TABLE 1

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys. Arg |
| Residues that influence chain orientation | Gly, Pro |
| Aromatic | Trp, Tyr, Phe |

The subclass of an antibody in part determines secondary effector functions, such as complement activation or Fc receptor (FcR) binding and antibody dependent cell cytotoxicity (ADCC) (Huber, et al., Nature 229(5284): 419-20 (1971); Brunhouse, et al., Mol Immunol 16(11): 907-17 (1979)). In identifying the optimal type of antibody for a particular application, the effector functions of the antibodies can be taken into account. For example, hIgG1 antibodies have a relatively long half life, are very effective at fixing complement, and they bind to both FcγRI and FcγRII. In contrast, human IgG4 antibodies have a shorter half life, do not fix complement and have a lower affinity for the FcRs. Replacement of serine 228 with a proline (S228P) in the Fc region of IgG4 reduces heterogeneity observed with hIgG4 and extends the serum half life (Kabat, et al., “Sequences of proteins of immunological interest” 5.sup.th Edition (1991); Angal, et al., Mol Immunol 30(1): 105-8 (1993)). A second mutation that replaces leucine 235 with a glutamic acid (L235E) eliminates the residual FcR binding and complement binding activities (Alegre, et al., J Immunol 148(11): 3461-8 (1992)). The resulting antibody with both mutations is referred to as IgG4PE. The numbering of the hIgG4 amino acids was derived from EU numbering reference: Edelman, G. M. et al., Proc. Natl. Acad. USA, 63, 78-85 (1969). PMID: 5257969. In one embodiment of the present invention the ICOS antibody is an IgG4 isotype. In one embodiment, the ICOS antibody comprises an IgG4 Fc region comprising the replacement S228P and L235E may have the designation IgG4PE.

As used herein “ICOS-L” and “ICOS Ligand” are used interchangeably and refer to the membrane bound natural ligand of human ICOS. ICOS ligand is a protein that in humans is encoded by the ICOSLG gene. ICOSLG has also been designated as CD275 (cluster of differentiation 275). Pseudonyms for ICOS-L include B7RP-1 and B7-H2.

As used herein, an “agent directed to PD-1” or “agent directed to PD1” means any chemical compound or biological molecule capable of binding to PD1. In some embodiments, the agent directed to PD1 is a PD1 antagonist.

The term “PD1 binding protein” or “PD-1 binding protein” as used herein refers to antibodies and other protein constructs, such as domains, which are capable of binding to PD1. In some instances, the PD1 is human PD1. The term “PD1 binding protein” can be used interchangeably with “PD1 antigen binding protein.” Thus, as is understood in the art, anti-PD1 antibodies and/or PD1 antigen binding proteins would be considered PD1 binding proteins. As used herein, “antigen binding protein” is any protein, including but not limited to antibodies, domains and other constructs described herein, that binds to an antigen, such as PD1. As used herein “antigen binding portion” of a PD1 binding protein would include any portion of the PD1 binding protein capable of binding to PD1, including but not limited to, an antigen binding antibody fragment.

The protein Programmed Death 1 (PD-1) is an inhibitory member of the CD28 family of receptors, that also includes CD28, CTLA-4, ICOS and BTLA. PD-1 is expressed on activated B cells, T cells, and myeloid cells (Agata et al., supra; Okazaki et al. (2002) Curr. Opin. Immunol 14:391779-82; Bennett et al. (2003) J Immunol 170:711-8) The initial members of the family, CD28 and ICOS, were discovered by functional effects on augmenting T cell proliferation following the addition of monoclonal antibodies (Hutloff et al. (1999) Nature 397:263-266; Hansen et al. (1980) Immunogenics 10:247-260). PD-1 was discovered through screening for differential expression in apototic cells (Ishida et al. (1992) EMBO J 11:3887-95) The other members of the family, CTLA-4, and BTLA were discovered through screening for differential expression in cytotoxic T lymphocytes and TH1 cells, respectively. CD28, ICOS and CTLA-4 all have an unpaired cysteine residue allowing for homodimerization. In contrast, PD-1 is suggested to exist as a monomer, lacking the unpaired cysteine residue characteristic in other CD28 family members. PD-1 antibodies and methods of using in treatment of disease are described in U.S. Pat. Nos.: U.S. Pat. Nos. 7,595,048; 8,168,179; 8,728,474; 7,722,868; 8,008,449; 7,488,802; 7,521,051; 8,088,905; 8,168,757; 8,354,509; and US Publication Nos. US20110171220; US20110171215; and US20110271358. Combinations of CTLA-4 and PD-1 antibodies are described in U.S. Pat. No. 9,084,776.

In some embodiments, the agent directed to PD1 is a PD1 antagonist and blocks binding of PD-L1 expressed on a cancer cell to PD-1 expressed on an immune cell (T cell, B cell or NKT cell) and may also block binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. Human PD-1 amino acid sequences can be found in NCBI Locus No.: NP_005009. The amino acid sequence in NCBI Locus No.: NP_005009 is reproduced below:

(SEQ ID NO: 11)
mqipqapwpv vwavlqlgwr pgwfldspdr pwnpptfspa
llvvtegdna tftcsfsnts esfvlnwyrm spsnqtdkla
afpedrsqpg qdcrfrvtql pngrdfhmsv vrarrndsgt
ylcgaislap kaqikeslra elrvterrae vptahpspsp
rpagqfqtlv vgvvggllgs lvllvwvlav icsraargti
garrtgqplk edpsavpvfs vdygeldfqw rektpeppvp
cvpeqteyat ivfpsgmgts sparrgsadg prsaqplrpe
dghcswpl

Human PD-L1 and PD-L2 amino acid sequences can be found in NCBI Locus No.: NP_054862 and NP_079515, respectively.
The amino acid sequence in NCBI Locus No.: NP_054862 is reproduced below:

| (SEQ ID NO: 12) | | | | | |
|---|---|---|---|---|---|
| mrifavfifm | tywhllnaft | vtvpkdlyvv | eygsnmtiec | kfpvekqldl | aalivyweme |
| dkniiqfvhg | eedlkvqhss | yrqrarllkd | qlslgnaalq | itdvklqdag | vyrcmisygg |
| adykritvkv | napynkinqr | ilvvdpvtse | heltcqaegy | pkaeviwtss | dhqvlsgktt |
| ttnskreekl | fnvtstlrin | tttneifyct | frrldpeenh | taelvipelp | lahppnerth |
| lvilgaillc | lgvaltfifr | lrkgrmmdvk | kcgiqdtnsk | kqsdthleet | |

The amino acid sequence in NCBI Locus No.: NP_079515 is reproduced below:

| (SEQ ID NO: 13) | | | | | |
|---|---|---|---|---|---|
| miflllmlsl | elqlhqiaal | ftvtvpkely | iiehgsnvtl | ecnfdtgshv | nlgaitaslq |
| kvendtsphr | eratlleeql | plgkasfhip | qvqvrdegqy | qciiiygvaw | dykyltlkvk |
| asyrkinthi | lkvpetdeve | ltcqatgypl | aevswpnvsv | pantshsrtp | eglyqvtsvl |
| rlkpppgrnf | scvfwnthvr | eltlasidlq | sqmeprthpt | wllhifipfc | iiafifiatv |
| ialrkqlcqk | lysskdttkr | pvtttkrevn | sai | | |

Agents directed to PD-1 in any of the aspects or embodiments of the present invention include a monoclonal antibody (mAb), or antigen binding fragment thereof, which specifically binds to PD-1. In some embodiments, the mAb to PD-1 specifically binds to human PD-1. The mAb may be a human antibody, a humanized antibody or a chimeric antibody, and may include a human constant region. In some embodiments, the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

Examples of mAbs that bind to human PD-1, and useful in the various aspects and embodiments of the present invention, are described in U.S. Pat. Nos. 8,552,154; 8,354,509; 8,168,757; 8,008,449; 7,521,051; 7,488,802; WO2004072286; WO2004056875; and WO2004004771.

Other PD-1 binding proteins useful in any of the aspects and embodiments of the present invention include an immunoadhesin that specifically binds to PD-1, and preferably specifically binds to human PD-1, e.g., a fusion protein containing the extracellular or PD-1 binding portion of PD-L1 or PD-L2 fused to a constant region such as an Fc region of an immunoglobulin molecule. Examples of immunoadhesin molecules that specifically bind to PD-1 are described in WO2010027827 and WO2011066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

OPDIVO/nivolumab is a fully human monoclonal antibody marketed by Bristol Myers Squibb directed against the negative immunoregulatory human cell surface receptor PD-1 (programmed death-1 or programmed cell death-1/PCD-1) with immunopotentiation activity. Nivolumab binds to and blocks the activation of PD-1, an Ig superfamily transmembrane protein, by its ligands PD-L1 and PD-L2, resulting in the activation of T-cells and cell-mediated immune responses against tumor cells or pathogens. Activated PD-1 negatively regulates T-cell activation and effector function through the suppression of P13k/Akt pathway activation. Other names for nivolumab include: BMS-936558, MDX-1106, and ONO-4538. The amino acid sequence for nivolumab and methods of using and making are disclosed in U.S. Pat. No. 8,008,449.

KEYTRUDA/pembrolizumab is an anti-PD-1 antibodies marketed for the treatment of lung cancer by Merck. The amino acid sequence of pembrolizumab and methods of using are disclosed in U.S. Pat. No. 8,168,757.

By “agent directed to PD-L1” is meant any chemical compound or biological molecule capable of binding to PD-L1. In some embodiments, the agent directed to PD-L1 is a PD-L1 binding protein.

The term “PDL1 binding protein” or “PD-L1 binding protein” as used herein refers to antibodies and other protein constructs, such as domains, which are capable of binding to PD-L1. In some instances, the PD-L1 is human PD1. The term “PD-L1 binding protein” can be used interchangeably with “PD-L1 antigen binding protein.” Thus, as is understood in the art, anti-PD-L1 antibodies and/or PD-L1 antigen binding proteins would be considered PD-L1 binding proteins. As used herein, “antigen binding protein” is any protein, including but not limited to antibodies, domains and other constructs described herein, that binds to an antigen, such as PD-L1. As used herein “antigen binding portion” of a PD-L1 binding protein would include any portion of the PD-L1 binding protein capable of binding to PD-L1, including but not limited to, an antigen binding antibody fragment.

In some embodiments, the agent directed to PD-L1 is a PD1 antagonist and blocks binding of PD-L1 expressed on a cancer cell to PD-1 expressed on an immune cell (T cell, B cell or NKT cell) and may also block binding of PD-L2 expressed on a cancer cell to the immune-cell expressed PD-1.

PD-L1 is a B7 family member that is expressed on many cell types, including APCs and activated T cells (Yamazaki et al. (2002) J. Immunol. 169:5538). PD-L1 binds to both PD-1 and B7-1. Both binding of T-cell-expressed B7-1 by PD-L1 and binding of T-cell-expressed PD-L1 by B7-1 result in T cell inhibition (Butte et al. (2007) Immunity 27:111). There is also evidence that, like other B7 family members, PD-L1 can also provide costimulatory signals to T cells (Subudhi et al. (2004) J. Clin. Invest. 113:694; Tamura et al. (2001) Blood 97:1809). PD-L1 (human PD-L1 cDNA is composed of the base sequence shown by EMBL/GenBank Acc. No. AF233516 and mouse PD-L1 cDNA is composed of the base sequence shown by NM.sub.—021893) that is a ligand of PD-1 is expressed in so-called antigen-presenting cells (APCs) such as activated monocytes and dendritic cells (Journal of Experimental Medicine (2000), vol. 19, issue 7, p 1027-1034). These cells present interaction molecules that induce a variety of immuno-inductive signals to T lymphocytes, and PD-L1 is one of these molecules that induce the inhibitory signal by PD-1. It has been revealed that PD-L1 ligand stimulation suppressed the activation (cellular proliferation and induction of various cytokine production) of PD-1 expressing T lymphocytes. PD-L1 expression has been confirmed in not only immunocompetent cells but also a certain kind of tumor cell lines (cell lines derived from monocytic leukemia, cell lines derived from mast cells, cell lines derived from hepatic carcinomas, cell lines derived from neuroblasts, and cell lines derived from breast carcinomas) (Nature Immunology (2001), vol. 2, issue 3, p. 261-267).

Anti-PD-L1 antibodies and methods of making the same are known in the art. Such antibodies to PD-L1 may be polyclonal or monoclonal, and/or recombinant, and/or humanized, and/or fully human. PD-L1 antibodies are in development as immuno-modulatory agents for the treatment of cancer.

Exemplary PD-L1 antibodies are disclosed in U.S. Pat. Nos. 9,212,224; 8,779,108; 8,552,154; 8,383,796; 8,217,149; US Patent Publication No. 20110280877; WO2013079174; and WO2013019906. Additional exemplary antibodies to PD-L1 (also referred to as CD274 or B7-H1) and methods for use are disclosed in U.S. Pat. Nos. 8,168,179; 7,943,743; 7,595,048; WO2014055897; WO2013019906; and WO2010077634. Specific anti-human PD-L1 monoclonal antibodies useful as a PD-1 antagonist in the treatment method, medicaments and uses of the present invention include MPDL3280A, BMS-936559, MEDI4736, MSB0010718C.

Atezolizumab is a fully humanized monoclonal anti-PD-L1 antibody commercially available as TECENTRIQ. Atezolizumab is indicated for the treatment of some locally advanced or metastatic urothelial carcinomas. Atezolizumab blocks the interaction of PD-L1 with PD-1 and CD80.

Durvalumab (previously known as MEDI4736) is a human monoclonal antibody directed against PD-L1. Durvalumab blocks the interaction of PD-L1 with PD-1 and CD80. Durvalumab is commercially available as IMFINZI™.

Antibodies to PD-L1 (also referred to as CD274 or B7-H1) and methods for use are disclosed in U.S. Pat. Nos. 7,943,743; 8,383,796; US20130034559, WO2014055897, U.S. Pat. Nos. 8,168,179; and 7,595,048. PD-L1 antibodies are in development as immuno-modulatory agents for the treatment of cancer.

As used herein the term “agonist” refers to an antigen binding protein including but not limited to an antibody, which upon contact with a co-signalling receptor causes one or more of the following (1) stimulates or activates the receptor, (2) enhances, increases or promotes, induces or prolongs an activity, function or presence of the receptor and/or (3) enhances, increases, promotes or induces the expression of the receptor. Agonist activity can be measured in vitro by various assays know in the art such as, but not limited to, measurement of cell signalling, cell proliferation, immune cell activation markers, cytokine production. Agonist activity can also be measured in vivo by various assays that measure surrogate end points such as, but not limited to the measurement of T cell proliferation or cytokine production.

As used herein the term “antagonist” refers to an antigen binding protein including but not limited to an antibody, which upon contact with a co-signalling receptor causes one or more of the following (1) attenuates, blocks or inactivates the receptor and/or blocks activation of a receptor by its natural ligand, (2) reduces, decreases or shortens the activity, function or presence of the receptor and/or (3) reduces, descrease, abrogates the expression of the receptor. Antagonist activity can be measured in vitro by various assays know in the art such as, but not limited to, measurement of an increase or decrease in cell signalling, cell proliferation, immune cell activation markers, cytokine production. Antagonist activity can also be measured in vivo by various assays that measure surrogate end points such as, but not limited to the measurement of T cell proliferation or cytokine production.

As used herein the term “cross competes for binding” refers to any agent such as an antibody that will compete for binding to a target with any of the agents of the present invention. Competition for binding between two antibodies can be tested by various methods known in the art including Flow cytometry, Meso Scale Discovery and ELISA. Binding can be measured directly, meaning two or more binding proteins can be put in contact with a co-signalling receptor and bind may be measured for one or each. Alternatively, binding of molecules or interest can be tested against the binding or natural ligand and quantitatively compared with each other.

The term “binding protein” as used herein refers to antibodies and other protein constructs, such as domains, which are capable of binding to an antigen.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanized, multispecific antibodies, including bispecific antibodies, and heteroconjugate antibodies; a single variable domain (e.g., V_{H}, V_{HH}, VL, domain antibody (dAb™)), antigen binding antibody fragments, Fab, F(ab′)₂, Fv, disulphide linked Fv, single chain Fv, disulphide-linked scFv, diabodies, TANDABS™, etc. and modified versions of any of the foregoing.

Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an avimer or an EGF domain.

The term “domain” refers to a folded protein structure which retains its tertiary structure independent of the rest of the protein. Generally domains are responsible for discrete functional properties of proteins and in many cases may be added, removed or transferred to other proteins without loss of function of the remainder of the protein and/or of the domain.

The term “single variable domain” refers to a folded polypeptide domain comprising sequences characteristic of antibody variable domains. It therefore includes complete antibody variable domains such as V_{H}, V_{HH} and V_{L} and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A single variable domain is capable of binding an antigen or epitope independently of a different variable region or domain. A “domain antibody” or “dAb™” may be considered the same as a “single variable domain”. A single variable domain may be a human single variable domain, but also includes single variable domains from other species such as rodent nurse shark and Camelid V_{HH} dAbs™. Camelid V_{HH} are immunoglobulin single variable domain polypeptides that are derived from species including camel, llama, alpaca, dromedary, and guanaco, which produce heavy chain antibodies naturally devoid of light chains. Such V_{HH} domains may be humanized according to standard techniques available in the art, and such domains are considered to be “single variable domains”. As used herein V_{H} includes camelid V_{HH} domains.

An antigen binding fragment may be provided by means of arrangement of one or more CDRs on non-antibody protein scaffolds. “Protein Scaffold” as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions.

The protein scaffold may be an Ig scaffold, for example an IgG, or IgA scaffold. The IgG scaffold may comprise some or all the domains of an antibody (i.e. CH1, CH2, CH3, V_{H}, V_{L}). The antigen binding protein may comprise an IgG scaffold selected from IgG1, IgG2, IgG3, IgG4 or IgG4PE. For example, the scaffold may be IgG1. The scaffold may consist of, or comprise, the Fc region of an antibody, or is a part thereof.

Affinity is the strength of binding of one molecule, e.g. an antigen binding protein of the invention, to another, e.g. its target antigen, at a single binding site. The binding affinity of an antigen binding protein to its target may be determined by equilibrium methods (e.g. enzyme-linked immunoabsorbent assay (ELISA) or radioimmunoassay (RIA)), or kinetics (e.g. BIACORE™ analysis). For example, the BIACORE™ methods described in Example 5 may be used to measure binding affinity.

Avidity is the sum total of the strength of binding of two molecules to one another at multiple sites, e.g. taking into account the valency of the interaction.

By “isolated” it is intended that the molecule, such as an antigen binding protein or nucleic acid, is removed from the environment in which it may be found in nature. For example, the molecule may be purified away from substances with which it would normally exist in nature. For example, the mass of the molecule in a sample may be 95% of the total mass.

The term “expression vector” as used herein means an isolated nucleic acid which can be used to introduce a nucleic acid of interest into a cell, such as a eukaryotic cell or prokaryotic cell, or a cell free expression system where the nucleic acid sequence of interest is expressed as a peptide chain such as a protein. Such expression vectors may be, for example, cosmids, plasmids, viral sequences, transposons, and linear nucleic acids comprising a nucleic acid of interest. Once the expression vector is introduced into a cell or cell free expression system (e.g., reticulocyte lysate) the protein encoded by the nucleic acid of interest is produced by the transcription/translation machinery. Expression vectors within the scope of the disclosure may provide necessary elements for eukaryotic or prokaryotic expression and include viral promoter driven vectors, such as CMV promoter driven vectors, e.g., pcDNA3.1, pCEP4, and their derivatives, Baculovirus expression vectors, Drosophila expression vectors, and expression vectors that are driven by mammalian gene promoters, such as human Ig gene promoters. Other examples include prokaryotic expression vectors, such as T7 promoter driven vectors, e.g., pET41, lactose promoter driven vectors and arabinose gene promoter driven vectors. Those of ordinary skill in the art will recognize many other suitable expression vectors and expression systems.

The term “recombinant host cell” as used herein means a cell that comprises a nucleic acid sequence of interest that was isolated prior to its introduction into the cell. For example, the nucleic acid sequence of interest may be in an expression vector while the cell may be prokaryotic or eukaryotic. Exemplary eukaryotic cells are mammalian cells, such as but not limited to, COS-1, COS-7, HEK293, BHK21, CHO, BSC-1, HepG2, 653, SP2/0, NS0, 293, HeLa, myeloma, lymphoma cells or any derivative thereof. Most preferably, the eukaryotic cell is a HEK293, NS0, SP2/0, or CHO cell. E. coli is an exemplary prokaryotic cell. A recombinant cell according to the disclosure may be generated by transfection, cell fusion, immortalization, or other procedures well known in the art. A nucleic acid sequence of interest, such as an expression vector, transfected into a cell may be extrachromasomal or stably integrated into the chromosome of the cell.

A “chimeric antibody” refers to a type of engineered antibody which contains a naturally-occurring variable region (light chain and heavy chains) derived from a donor antibody in association with light and heavy chain constant regions derived from an acceptor antibody.

A “humanized antibody” refers to a type of engineered antibody having its CDRs derived from a non-human donor immunoglobulin, the remaining immunoglobulin-derived parts of the molecule being derived from one or more human immunoglobulin(s). In addition, framework support residues may be altered to preserve binding affinity (see, e.g., Queen et al. Proc. Natl Acad Sci USA, 86:10029-10032 (1989), Hodgson, et al., Bio/Technology, 9:421 (1991)). A suitable human acceptor antibody may be one selected from a conventional database, e.g., the KABAT™ database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies—see, for example, EP-A-0239400 and EP-A-054951.

The term “fully human antibody” includes antibodies having variable and constant regions (if present) derived from human germline immunoglobulin sequences. The human sequence antibodies of the invention may include amino acid residues not encoded by human germline immunoglobulin sequences (e.g., mutations introduced by random or site-specific mutagenesis in vitro or by somatic mutation in vivo). Fully human antibodies comprise amino acid sequences encoded only by polynucleotides that are ultimately of human origin or amino acid sequences that are identical to such sequences. As meant herein, antibodies encoded by human immunoglobulin-encoding DNA inserted into a mouse genome produced in a transgenic mouse are fully human antibodies since they are encoded by DNA that is ultimately of human origin. In this situation, human immunoglobulin-encoding DNA can be rearranged (to encode an antibody) within the mouse, and somatic mutations may also occur. Antibodies encoded by originally human DNA that has undergone such changes in a mouse are fully human antibodies as meant herein. The use of such transgenic mice makes it possible to select fully human antibodies against a human antigen. As is understood in the art, fully human antibodies can be made using phage display technology wherein a human DNA library is inserted in phage for generation of antibodies comprising human germline DNA sequence.

The term “donor antibody” refers to an antibody that contributes the amino acid sequences of its variable regions, CDRs, or other functional fragments or analogs thereof to a first immunoglobulin partner. The donor, therefore, provides the altered immunoglobulin coding region and resulting expressed altered antibody with the antigenic specificity and neutralising activity characteristic of the donor antibody.

The term “acceptor antibody” refers to an antibody that is heterologous to the donor antibody, which contributes all (or any portion) of the amino acid sequences encoding its heavy and/or light chain framework regions and/or its heavy and/or light chain constant regions to the first immunoglobulin partner. A human antibody may be the acceptor antibody.

The terms “V_{H}” and “V_{L}” are used herein to refer to the heavy chain variable region and light chain variable region respectively of an antigen binding protein.

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and full length antibody sequences are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 5th Ed., U.S. Department of Health and Human Services, National Institutes of Health (1991).

It will be apparent to those skilled in the art that there are alternative numbering conventions for amino acid residues in variable domain sequences and full length antibody sequences. There are also alternative numbering conventions for CDR sequences, for example those set out in Chothia et al. (1989) Nature 342: 877-883. The structure and protein folding of the antibody may mean that other residues are considered part of the CDR sequence and would be understood to be so by a skilled person.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

“Percent identity” between a query nucleic acid sequence and a subject nucleic acid sequence is the “Identities” value, expressed as a percentage, that is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off.

“Percent identity” between a query amino acid sequence and a subject amino acid sequence is the “Identities” value, expressed as a percentage, that is calculated by the BLASTP algorithm when a subject amino acid sequence has 100% query coverage with a query amino acid sequence after a pair-wise BLASTP alignment is performed. Such pair-wise BLASTP alignments between a query amino acid sequence and a subject amino acid sequence are performed by using the default settings of the BLASTP algorithm available on the National Center for Biotechnology Institute's website with the filter for low complexity regions turned off.

The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

The % identity may be determined across the entire length of the query sequence, including the CDR(s). Alternatively, the % identity may exclude the CDR(s), for example the CDR(s) is 100% identical to the subject sequence and the % identity variation is in the remaining portion of the query sequence, so that the CDR sequence is fixed/intact.

In one aspect, methods of treating cancer in a patient in need thereof, comprising administering to the patient an effective amount of an agent directed to human ICOS and an effective amount of an agent directed to human PD1 or human PD-L1 sequentially are provided. In one embodiment, administration of the agent directed to human ICOS is followed by administration of the agent directed to human PD1 or human PD-L1. In one embodiment, the agent directed to human PD1 or human PD-L1 is administered concurrently with an agent directed to human ICOS in the phase following administration of the agent directed to human ICOS.

In another aspect, administration of the agent directed to human PD1 or human PD-L1 is followed by administration of the agent directed to human ICOS. In one embodiment, the agent directed to human ICOS is an anti-ICOS antibody or antigen binding portion thereof. In one embodiment, the agent directed to human ICOS is administered concurrently with an agent directed to human PD1 or human PD-L1 in the phase following administration of the agent directed to human PD1 or human PD-L1.

In one aspect, an anti-ICOS antibody or antigen binding fragment thereof and an anti-PD 1 antibody or antigen binding fragment thereof for sequential use in treating cancer in a human in need thereof are provided. In one embodiment, administration of the anti-ICOS antibody or antigen binding fragment thereof is followed by administration of the anti-PD1 antibody or antigen binding fragment thereof. In another embodiment, administration of the anti-PD1 antibody or antigen binding fragment thereof is followed by administration of the anti-ICOS antibody or antigen binding fragment thereof.

In one aspect, an anti-ICOS antibody or antigen binding fragment thereof and an anti-PD-L1 antibody or antigen binding fragment thereof for sequential use in treating cancer in a human in need thereof are provided. In one embodiment, administration of the anti-ICOS antibody or antigen binding fragment thereof is followed by administration of the anti-PD-L1 antibody or antigen binding fragment thereof. In another embodiment, administration of the anti-PD-L1 antibody or antigen binding fragment thereof is followed by administration of the anti-ICOS antibody or antigen binding fragment thereof.

In another aspect, use of an anti-ICOS antibody or antigen binding portion thereof and an anti-PD1 antibody or antigen binding portion thereof in the manufacture of a medicament for the treatment of cancer is provided, wherein the anti-ICOS antibody or antigen binding portion thereof and an anti-PD1 antibody or antigen binding portion thereof are sequentially administered, and wherein administration of the anti-ICOS antibody or antigen binding portion thereof is followed by administration of the anti-PD1 antibody or antigen binding portion thereof.

In another aspect, use of an anti-ICOS antibody or antigen binding portion thereof and an anti-PDL1 antibody or antigen binding portion thereof in the manufacture of a medicament for the treatment of cancer is provided, wherein the anti-ICOS antibody or antigen binding portion thereof and an anti-PDL1 antibody or antigen binding portion thereof are sequentially administered, and wherein administration of the anti-ICOS antibody or antigen binding portion thereof is followed by administration of the anti-PDL1 antibody or antigen binding portion thereof.

The present invention also provides polynucleotides encoding anti-ICOS antibodies, anti-PD1 antibodies, anti-PDL1 antibodies, or antigen binding portion of any one of said antibodies, of the present invention. In one embodiment, host cells are provided comprising polynucleotides encoding anti-ICOS antibodies, anti-PD1 antibodies, or anti-PDL1 antibodies, or antigen binding portions of any one of said antibodies, of the present invention. The present invention also provides methods of making an anti-ICOS antibody, anti-PD1 antibody, anti-PDL1 antibody, or an antigen binding portion of said antibody, comprising the steps of a) culturing host cell comprising a polynucleotide encoding an anti-ICOS antibody, anti-PD1 antibody, or anti-PDL1 antibody or an antigen binding portion of said antibody of the present invention under suitable conditions to express said anti-ICOS antibody, anti-PD1 antibody, or anti-PDL1 antibody or antigen binding portion of said antibody; and b) isolating said anti-ICOS, anti-PD1, or anti-PDL1 antibody or antigen binding portion of said antibody.

In another aspect, a polynucleotide encoding an anti-ICOS antibody or antigen binding portion thereof is provided, wherein the anti-ICOS antibody or antigen binding portion thereof is sequentially administered to a cancer patient with an anti-PD1 antibody or antigen binding portion thereof, and wherein administration of the anti-ICOS antibody or antigen binding portion thereof is followed by administration of the anti-PD1 antibody or antigen binding portion thereof.

In another aspect, a polynucleotide encoding an anti-ICOS antibody or antigen binding portion thereof is provided, wherein the anti-ICOS antibody or antigen binding portion thereof is sequentially administered to a cancer patient with an anti-PDL1 antibody or antigen binding portion thereof, and wherein administration of the anti-ICOS antibody or antigen binding portion thereof is followed by administration of the anti-PDL1 antibody or antigen binding portion thereof.

In yet another aspect, a polynucleotide encoding an anti-PD1 antibody or antigen binding portion thereof is provided, wherein the anti-PD1 antibody or antigen binding portion thereof is sequentially administered to a cancer patient with an anti-ICOS antibody or antigen binding portion thereof, and wherein administration of the anti-ICOS antibody or antigen binding portion thereof is followed by administration of the anti-PD1 antibody or antigen binding portion thereof.

In still another aspect, a polynucleotide encoding an anti-PDL1 antibody or antigen binding portion thereof is provided, wherein the anti-PDL1 antibody or antigen binding portion thereof is sequentially administered to a cancer patient with an anti-ICOS antibody or antigen binding portion thereof, and wherein administration of the anti-ICOS antibody or antigen binding portion thereof is followed by administration of the anti-PDL1 antibody or antigen binding portion thereof.

In another aspect, a vector comprising the polynucleotide of any one of the aspects herein is provided. In another aspect, a host cell comprising the vector of any one of the aspects herein is provided.

In yet another aspect, a method of making an anti-ICOS antibody or antigen binding portion thereof is provided, the method comprising a) culturing a host cell comprising the polynucleotide of any one of the aspects herein under suitable conditions to express the anti-ICOS antibody or antigen binding portion thereof; and b) isolating said anti-ICOS antibody or antigen binding portion thereof.

In another aspect, a method of making an anti-PD1 antibody or antigen binding portion thereof is provided, the method comprising a) culturing a host cell comprising the polynucleotide of any one of the aspects herein under suitable conditions to express the anti-PD 1 antibody or antigen binding portion thereof, and b) isolating said anti-PD1 antibody or antigen binding portion thereof.

In still another aspect, a method of making an anti-PDL1 antibody or antigen binding portion thereof is provided, the method comprising a) culturing a host cell comprising the polynucleotide of any one of the aspects herein under suitable conditions to express the anti-PDL1 antibody or antigen binding portion thereof, and b) isolating said anti-PDL1 antibody or antigen binding portion thereof.

In one embodiment of any one of the aspects herein, the anti-ICOS antibody is an ICOS agonist. In one embodiment, the anti-ICOS antibody comprises a V_{H} domain comprising an amino acid sequence at least 90% identical to the amino acid sequence set forth in SEQ ID NO:7; and a V_{L} domain comprising an amino acid sequence at least 90% identical to the amino acid sequence as set forth in SEQ ID NO:8. In another embodiment, the anti-ICOS antibody comprises a V_{H} domain comprising the amino acid sequence set forth in SEQ ID NO:7 and a V_{L} domain comprising the amino acid sequence as set forth in SEQ ID NO:8. In one embodiment, the anti-ICOS antibody comprises one or more of: CDRH1 as set forth in SEQ ID NO: 1; CDRH2 as set forth in SEQ ID NO:2; CDRH3 as set forth in SEQ ID NO:3; CDRL1 as set forth in SEQ ID NO:4; CDRL2 as set forth in SEQ ID NO:5 and/or CDRL3 as set forth in SEQ ID NO:6 or a direct equivalent of each CDR wherein a direct equivalent has no more than two amino acid substitutions in said CDR.

In one embodiment of any one of the aspects herein, the agent directed to human PD1 is an anti-PD1 antibody or antigen binding portion thereof. In one embodiment, the anti-PD1 antibody is a PD1 antagonist. In one embodiment, the anti-PD1 antibody is pembrolizumab. In another embodiment, the anti-PD1 antibody is nivolumab. In one embodiment of any one of the aspects herein, the agent directed to human PD-L1 is an anti-PD-L1 antibody or antigen binding portion thereof. In one embodiment, the anti-PD-L1 antibody is a PD1 antagonist. In one embodiment, the anti-PD-L1 antibody is durvalumab.

In one embodiment of any one of the aspects herein, the agent directed to human ICOS is administered for 2, 3, 4, 5, 6, 7, 8, 9, 10, 11, 12, 13, 14, 15, 16, 17, 18, 19, 20, 21, 22, 23, 24, 25, 26, 27, 28, 29, or 30 consecutive days. In one embodiment of any one of the aspects herein, the agent directed to human PD1 or human PD-L1 is administered for 2, 3, 4, 5, 6, 7, 8, 9, 10, 11, 12, 13, 14, 15, 16, 17, 18, 19, 20, 21, 22, 23, 24, 25, 26, 27, 28, 29, or 30 consecutive days.

In one aspect, the cancer is selected from the group consisting of colorectal cancer (CRC), gastric, esophageal, cervical, bladder, breast, head and neck, ovarian, melanoma, renal cell carcinoma (RCC), EC squamous cell, non-small cell lung carcinoma, mesothelioma, pancreatic, and prostate cancer.

In one aspect, the present invention provides a method of treating cancer in a human in need thereof, the method comprising administering to said human an anti-ICOS antibody or antigen binding fragment thereof and/or administering to said human an anti-PD1 antibody or antigen binding fragment thereof. In one embodiment, the anti-ICOS antibody or antigen binding fragment thereof induces T-cell proliferation, expansion, and tumor infiltration. In another embodiment, the anti-ICOS antibody or antigen binding fragment thereof increases PD-1 expression on a T-cell. In one embodiment, the anti-PD1 antibody or antigen binding fragment thereof increases ICOS expression on a T-cell. In one embodiment, the anti-ICOS antibody or antigen binding fragment thereof is an IgG4 isotype and reduces depletion of ICOS-positive T-cells. In another embodiment, the anti-ICOS antibody or antigen binding fragment thereof is an IgG4 isotype and results in increased anti-cancer efficacy when compared to an IgG1 isotype anti-ICOS antibody.

In another embodiment the cancer is selected from head and neck cancer, breast cancer, lung cancer, colon cancer, ovarian cancer, prostate cancer, gliomas, glioblastoma, astrocytomas, glioblastoma multiforme, Bannayan-Zonana syndrome, Cowden disease, Lhermitte-Duclos disease, inflammatory breast cancer, Wilm's tumor, Ewing's sarcoma, Rhabdomyosarcoma, ependymoma, medulloblastoma, kidney cancer, liver cancer, melanoma, pancreatic cancer, sarcoma, osteosarcoma, giant cell tumor of bone, thyroid cancer, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), and testicular cancer.

Some embodiments of the present invention further comprise administering at least one neo-plastic agent and/or at least one immunostimulatory agent to said human.

In one aspect the human has a solid tumor. In one aspect the tumor is selected from head and neck cancer, gastric cancer, melanoma, renal cell carcinoma (RCC), esophageal cancer, non-small cell lung carcinoma, prostate cancer, colorectal cancer, ovarian cancer and pancreatic cancer. In another aspect the human has a liquid tumor such as diffuse large B cell lymphoma (DLBCL), multiple myeloma, chronic lyphomblastic leukemia (CLL), follicular lymphoma, acute myeloid leukemia and chronic myelogenous leukemia.

The present disclosure also relates to a method for treating or lessening the severity of a cancer selected from: brain (gliomas), glioblastomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma, Hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

By the term “treating” and grammatical variations thereof as used herein, is meant therapeutic therapy. In reference to a particular condition, treating means: (1) to ameliorate the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy using the methods and/or compositions of the invention is also contemplated. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, the terms “cancer,” “neoplasm,” and “tumor” are used interchangeably and, in either the singular or plural form, refer to cells that have undergone a malignant transformation that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors.” Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent Bcell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphoma s(T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenstrom's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

As used herein the term “Compound A²” means an agent directed to human ICOS. In some embodiments, Compound A² is an antibody to human ICOS or the antigen binding 5 portion thereof. In some embodiments, Compound A² is an ICOS agonist. Suitably Compound A² means a humanized monoclonal antibody having a heavy chain variable region as set forth in SEQ ID NO:7 and a light chain variable region as set forth in SEQ ID NO:8.

As used herein the term “Compound B²” means an agent directed to human PD1 or an agent to directed to human PD-L1. In some embodiments, Compound B² is a PD1 antagonist. In some embodiments, Compound B² is an antibody to human PD1 or the antigen binding portion thereof. In some embodiments, Compound B² is an antibody to human PD-L1 or the antigen binding portion thereof. Suitably, Compound B² is nivolumab. Suitably, Compound B² is pembrolizumab.

Suitably, the combinations of this invention are administered within a “specified period”.

The term “specified period” and grammatical variations thereof, as used herein, means the interval of time between the administration of one of Compound A² and Compound B² and the other of Compound A² and Compound B².

Suitably, if the compounds are administered within a “specified period” and not administered simultaneously, they are both administered within about 24 hours of each other—in this case, the specified period will be about 24 hours; suitably they will both be administered within about 12 hours of each other—in this case, the specified period will be about 12 hours; suitably they will both be administered within about 11 hours of each other—in this case, the specified period will be about 11 hours; suitably they will both be administered within about 10 hours of each other—in this case, the specified period will be about 10 hours; suitably they will both be administered within about 9 hours of each other—in this case, the specified period will be about 9 hours; suitably they will both be administered within about 8 hours of each other—in this case, the specified period will be about 8 hours; suitably they will both be administered within about 7 hours of each other—in this case, the specified period will be about 7 hours; suitably they will both be administered within about 6 hours of each other—in this case, the specified period will be about 6 hours; suitably they will both be administered within about 5 hours of each other—in this case, the specified period will be about 5 hours; suitably they will both be administered within about 4 hours of each other—in this case, the specified period will be about 4 hours; suitably they will both be administered within about 3 hours of each other—in this case, the specified period will be about 3 hours; suitably they will be administered within about 2 hours of each other—in this case, the specified period will be about 2 hours; suitably they will both be administered within about 1 hour of each other—in this case, the specified period will be about 1 hour. As used herein, the administration of Compound A² and Compound B² in less than about 45 minutes apart is considered simultaneous administration.

Suitably, when the combination of the invention is administered for a “specified period”, the compounds will be co-administered for a “duration of time”.

The term “duration of time” and grammatical variations thereof, as used herein means that both compounds of the invention are administered for an indicated number of consecutive days. Unless otherwise defined, the number of consecutive days does not have to commence with the start of treatment or terminate with the end of treatment, it is only required that the number of consecutive days occur at some point during the course of treatment.

### Regarding “Specified Period” Administration:

Suitably, both compounds will be administered within a specified period for at least one day—in this case, the duration of time will be at least one day; suitably, during the course to treatment, both compounds will be administered within a specified period for at least 3 consecutive days—in this case, the duration of time will be at least 3 days; suitably, during the course to treatment, both compounds will be administered within a specified period for at least 5 consecutive days—in this case, the duration of time will be at least 5 days; suitably, during the course to treatment, both compounds will be administered within a specified period for at least 7 consecutive days—in this case, the duration of time will be at least 7 days; suitably, during the course to treatment, both compounds will be administered within a specified period for at least 14 consecutive days—in this case, the duration of time will be at least 14 days; suitably, during the course to treatment, both compounds will be administered within a specified period for at least 30 consecutive days—in this case, the duration of time will be at least 30 days.

Suitably, if the compounds are not administered during a “specified period”, they are administered sequentially. By the term “sequential administration”, and grammatical derivates thereof, as used herein is meant that one of Compound A² and Compound B² is administered for two or more consecutive days and the other of Compound A² and Compound B² is subsequently administered for two or more consecutive days. During the period of consecutive days in which Compound A² is administered, at least 1 dose, at least 2 doses, at least 3 doses, at least 4 doses, at least 5 doses, at least 6 doses, at least 7 doses, at least 8 doses, at least 9 doses, or at least 10 doses of Compound A² is administered. During the period of consecutive days in which Compound B² is administered, at least 1 dose, at least 2 doses, at least 3 doses, at least 4 doses, at least 5 doses, at least 6 doses, at least 7 doses, at least 8 doses, at least 9 doses, or at least 10 doses Compound B² is administered. During the period of consecutive days in which Compound A² is administered, Compound A² can be administered at least three times a day, at least twice a day, at least once a day, or less than once a day, e.g., once every 2 days, once every 3 days, once every week, once every 2 weeks, once every 3 weeks, or once every 4 weeks. During the period of consecutive days in which Compound B² is administered, Compound B² can be administered at least three times a day, at least twice a day, at least once a day, or less than once a day, e.g., once every 2 days, once every 3 days, once every week, once every 2 weeks, once every 3 weeks, or once every 4 weeks.

Also, contemplated herein is a drug holiday utilized between the sequential administration of one of Compound A² and Compound B² and the other of Compound A² and Compound B². As used herein, a drug holiday is a period of days after the sequential administration of one of Compound A² and Compound B² and before the administration of the other of Compound A² and Compound B² where neither Compound A² nor Compound B² is administered. Suitably the drug holiday will be a period of days selected from: 1 day, 2 days, 3 days, 4 days, 5 days, 6 days, 7 days, 8 days, 9 days, 10 days, 11 days, 12 days, 13 days and 14 days.

Sequential administration can also include one of Compound A² and Compound B² is administered for two or more consecutive days and then both of Compound A² and Compound B² is subsequently administered for two or more consecutive days. Sequential administration can include both of Compound A² and Compound B² being administered for two or more consecutive days and then one of Compound A² and Compound B² being subsequently administered for two or more consecutive days

### Regarding Sequential Administration:

Suitably, one of Compound A² and Compound B² is administered for from 1 to 30 consecutive days, followed by an optional drug holiday, followed by administration of the other of Compound A² and Compound B² for from 1 to 30 consecutive days. Suitably, one of Compound A² and Compound B² is administered for from 1 to 21 consecutive days, followed by an optional drug holiday, followed by administration of the other of Compound A² and Compound B² for from 1 to 21 consecutive days. Suitably, one of Compound A² and Compound B² is administered for from 1 to 14 consecutive days, followed by a drug holiday of from 1 to 14 days, followed by administration of the other of Compound A² and Compound B² for from 1 to 14 consecutive days. Suitably, one of Compound A² and Compound B² is administered for from 1 to 7 consecutive days, followed by a drug holiday of from 1 to 10 days, followed by administration of the other of Compound A² and Compound B² for from 1 to 7 consecutive days.

Suitably, Compound B² will be administered first in the sequence, followed by an optional drug holiday, followed by administration of Compound A². Suitably, Compound B² is administered for from 3 to 21 consecutive days, followed by an optional drug holiday, followed by administration of Compound A² for from 3 to 21 consecutive days. Suitably, Compound B² is administered for from 3 to 21 consecutive days, followed by a drug holiday of from 1 to 14 days, followed by administration of Compound A² for from 3 to 21 consecutive days. Suitably, Compound B² is administered for from 3 to 21 consecutive days, followed by a drug holiday of from 3 to 14 days, followed by administration of Compound A² for from 3 to 21 consecutive days. Suitably, Compound B² is administered for 21 consecutive days, followed by an optional drug holiday, followed by administration of Compound A² for 14 consecutive days. Suitably, Compound B² is administered for 14 consecutive days, followed by a drug holiday of from 1 to 14 days, followed by administration of Compound A² for 14 consecutive days. Suitably, Compound B² is administered for 7 consecutive days, followed by a drug holiday of from 3 to 10 days, followed by administration of Compound A² for 7 consecutive days. Suitably, Compound B² is administered for 3 consecutive days, followed by a drug holiday of from 3 to 14 days, followed by administration of Compound A² for 7 consecutive days. Suitably, Compound B² is administered for 3 consecutive days, followed by a drug holiday of from 3 to 10 days, followed by administration of Compound A² for 3 consecutive days.

It is understood that a “specified period” administration and a “sequential” administration can be followed by repeat dosing or can be followed by an alternate dosing protocol, and a drug holiday may precede the repeat dosing or alternate dosing protocol.

The methods of the present invention may also be employed with other therapeutic methods of cancer treatment.

Compound A² and Compound B² may be administered by any appropriate route. Suitable routes include oral, rectal, nasal, topical (including buccal and sublingual), intratumorally, vaginal, and parenteral (including subcutaneous, intramuscular, intravenous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that Compound A² and Compound B² may be compounded together in a pharmaceutical composition/formulation.

In one embodiment, one or more components of a combination of the invention are administered intravenously. In one embodiment, one or more components of a combination of the invention are administered orally. In another embodiment, one or more components of a combination of the invention are administered intratumorally. In another embodiment, one or more components of a combination of the invention are administered systemically, e.g., intravenously, and one or more other components of a combination of the invention are administered intratumorally. In any of the embodiments, e.g., in this paragraph, the components of the invention are administered as one or more pharmaceutical compositions.

In one aspect methods are provided for the treatment of cancer, comprising administering to a human in need thereof a therapeutically effective amount of (i) an anti-ICOS antibody or the antigen binding portion thereof, in addition to one of more diluents, vehicles, excipients and/or inactive ingredients, and (ii) an anti-PD1 antibody or the antigen binding portion thereof or an anti-PDL1 antibody or the antigen binding portion thereof, in addition to one of more diluents, vehicles, excipients and/or inactive ingredients. In one embodiment sequential administration of an anti-ICOS antibody or the antigen binding portion thereof and an anti-PD1 antibody or antigen binding portion thereof provides a synergistic effect compared to administration of either agent as monotherapy or concurrently. In one embodiment, sequential administration of an anti-ICOS antibody or the antigen binding portion thereof and an anti-PDL1 antibody or antigen binding portion thereof provides a synergistic effect compared to administration of either agent as monotherapy or concurrently.

In one embodiment, the anti-ICOS antibody or antigen binding portion thereof comprises a V_{H} domain comprising an amino acid sequence at least 90% identical to the amino acid sequence set forth in SEQ ID NO:7; and a V_{L} domain comprising an amino acid sequence at least 90% identical to the amino acid sequence as set forth in SEQ ID NO:8.

In one embodiment, methods of treating cancer are provided wherein the anti-ICOS antibody or antigen binding portion thereof is administered at a time interval selected from once every week, once every two weeks, once every three weeks, and once every four weeks. In another embodiment, the anti-PD1 antibody or antigen binding portion thereof or the anti-PDL1 antibody or antigen binding portion thereof is administered at a time interval selected from once every week, once every two weeks, once every three weeks, and once every four weeks. As is understood in the art the start of administration of either agent can be separated by an interstitial period. The interstitial period may be 12 hours, one to six days, one week, two weeks, three weeks, four weeks, five weeks, or six weeks. By way of example, an anti-ICOS antibody could be administered on Day 1 of treatment with an interstitial period of two weeks before the start of anti-PD1 antibody therapy which would start on Day 14. In one aspect, treatment with said anti-ICOS antibody could continue with administration of a single IV infusion at a time interval of, for example, every one, two, three or four weeks. Similarly, treatment with said anti-PD1 antibody could continue with administration of a single IV infusion at a time interval of, for example, every one, two, three or four weeks.

In one embodiment, the anti-ICOS antibody or antigen binding portion thereof is administered as an IV infusion. In one embodiment, the anti-PD1 antibody or antigen binding portion thereof is administered as an IV infusion. In one embodiment, the anti-PDL1 antibody or antigen binding portion thereof is administered as an IV infusion. In one aspect, the anti-ICOS antibody or antigen binding portion thereof is administered prior to the anti-PD1 antibody or the antigen binding portion thereof or the anti-PD1 antibody or the antigen binding portion thereof. In one embodiment, administration of the anti-PD1 antibody or antigen binding portion thereof or the anti-PDL1 antibody or antigen binding portion thereof is initiated at a time point selected from 1 week, 2 weeks, 3 weeks, and 4 weeks after the start of the administration of said anti-ICOS antibody or antigen binding portion thereof. In one aspect, the anti-PD1 antibody or antigen binding portion thereof or the anti-PDL1 antibody or antigen binding portion thereof is administered prior to the anti-ICOS antibody or the antigen binding portion thereof. In one embodiment, the interstitial period between the start of the anti-PD1 antibody or anti-PDL1 therapy and the start of the anti-ICOS antibody therapy is selected from 1 day, 1 week, 2 weeks, 3 weeks, 4 weeks, 5 weeks, and 6 weeks.

In one embodiment, the anti-ICOS antibody or antigen binding portion thereof and said anti-PD1 antibody or antigen binding portion thereof or anti-PDL1 antibody or antigen binding portion thereof are administered to said human until said human shows disease progression or unacceptable toxicity. In one embodiment, methods are provided for the treatment of cancer further comprising administering at least one anti neoplastic agent and/or at least one immuno-modulatory agent to said human.

Typically, any anti-neoplastic agent that has activity versus a susceptible tumor being treated may be co-administered in the treatment of cancer in the present invention. Examples of such agents can be found in Cancer Principles and Practice of Oncology by V. T. Devita, T. S. Lawrence, and S. A. Rosenberg (editors), 10^{th} edition (Dec. 5, 2014), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule or anti-mitotic agents such as diterpenoids and vinca alkaloids; platinum coordination complexes; alkylating agents such as nitrogen mustards, oxazaphosphorines, alkylsulfonates, nitrosoureas, and triazenes; antibiotic agents such as actinomycins, anthracyclins, and bleomycins; topoisomerase I inhibitors such as camptothecins; topoisomerase II inhibitors such as epipodophyllotoxins; antimetabolites such as purine and pyrimidine analogues and anti-folate compounds; hormones and hormonal analogues; signal transduction pathway inhibitors; non-receptor tyrosine kinase angiogenesis inhibitors; immunotherapeutic agents; proapoptotic agents; cell cycle signalling inhibitors; proteasome inhibitors; heat shock protein inhibitors; inhibitors of cancer metabolism; and cancer gene therapy agents such as genetically modified T cells.

Examples of a further active ingredient or ingredients for use in combination or co-administered with the present methods or combinations are anti-neoplastic agents. Examples of anti-neoplastic agents include, but are not limited to, chemotherapeutic agents; immuno-modulatory agents; immuno-modulators; and immunostimulatory adjuvants.

### EXAMPLES

The following examples illustrate various non-limiting aspects of this invention.

### Example 1

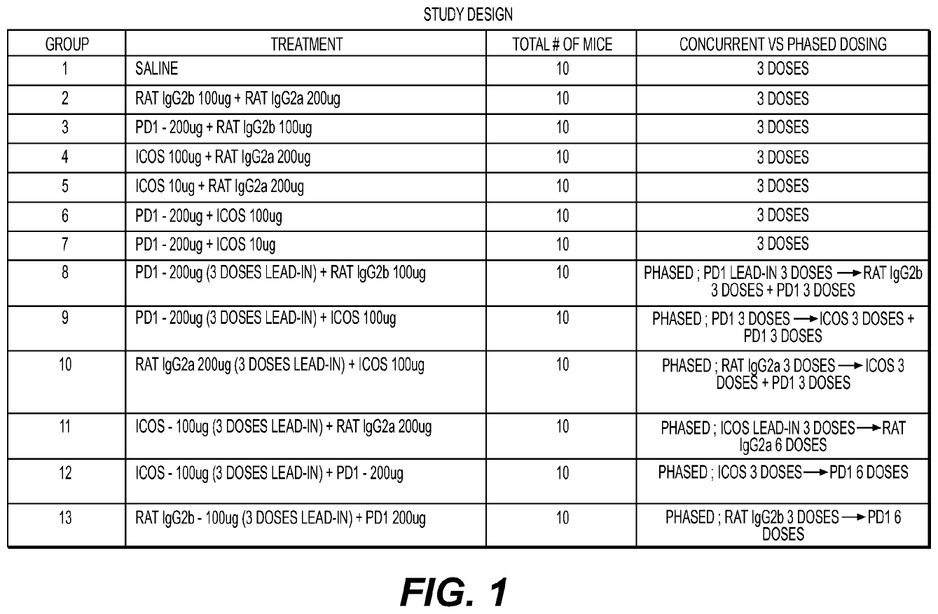

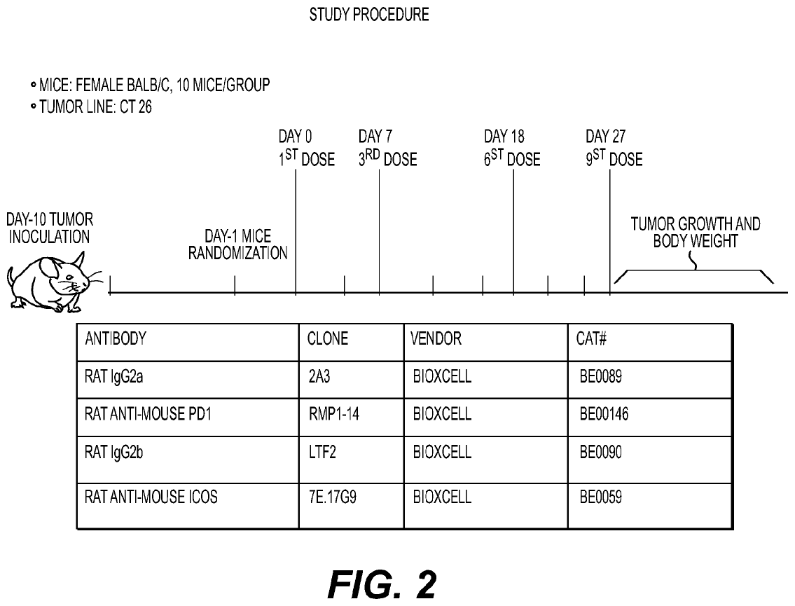

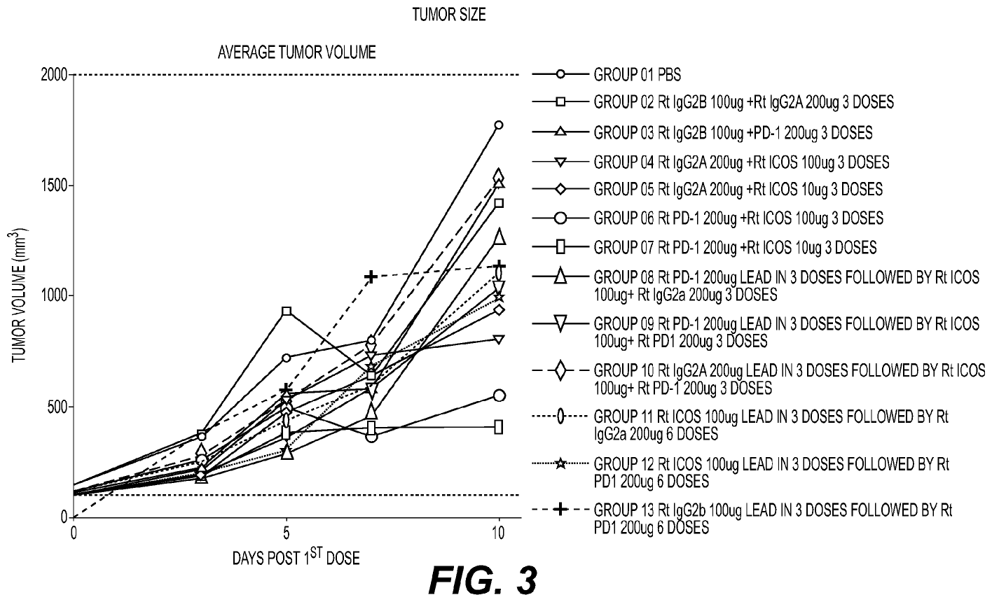

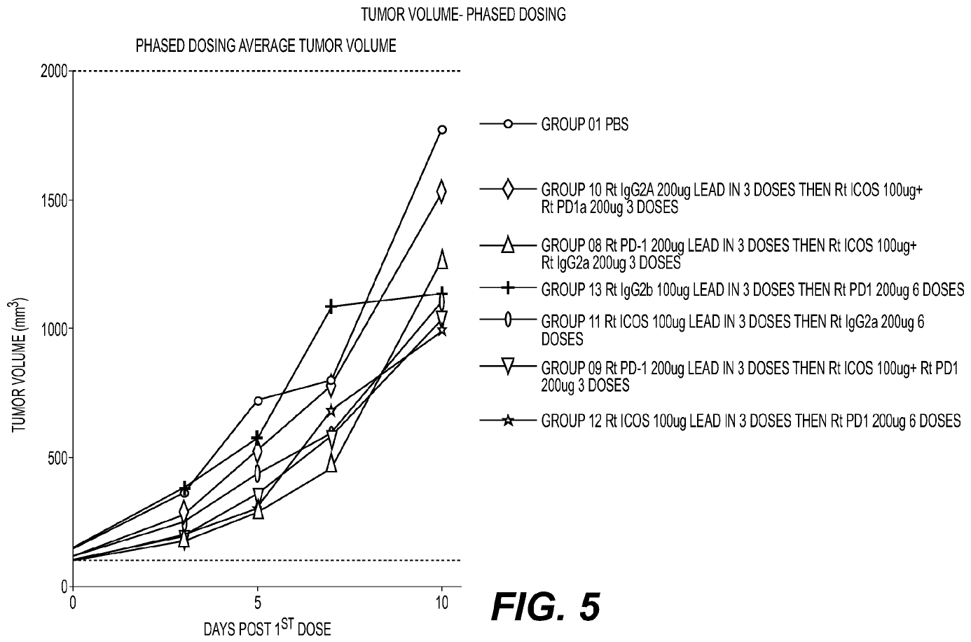

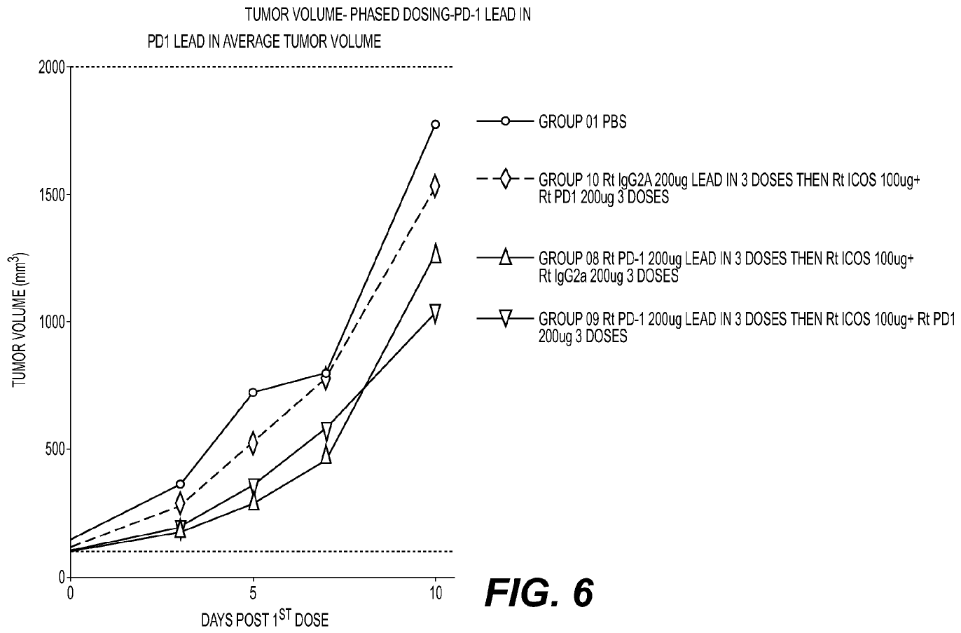

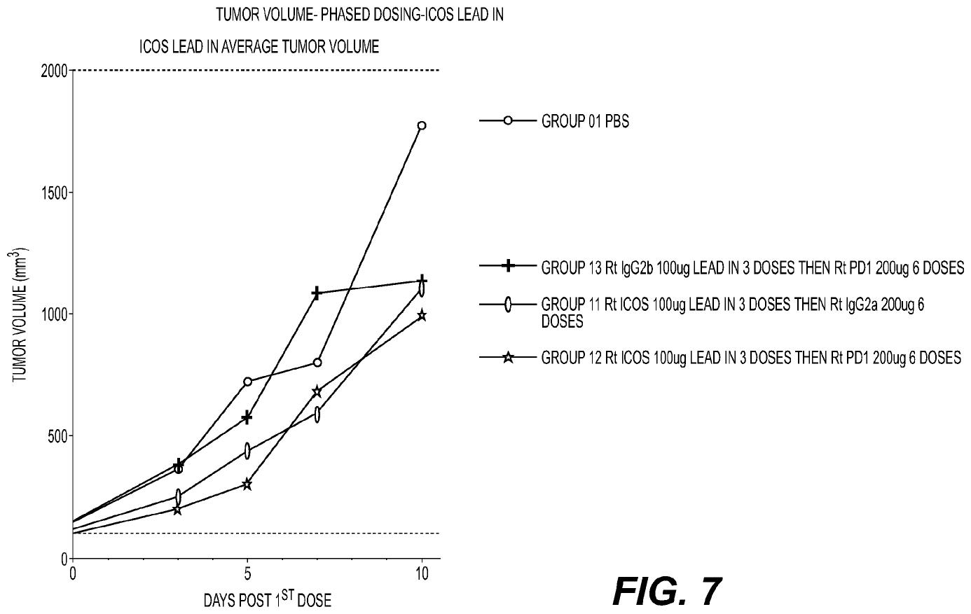

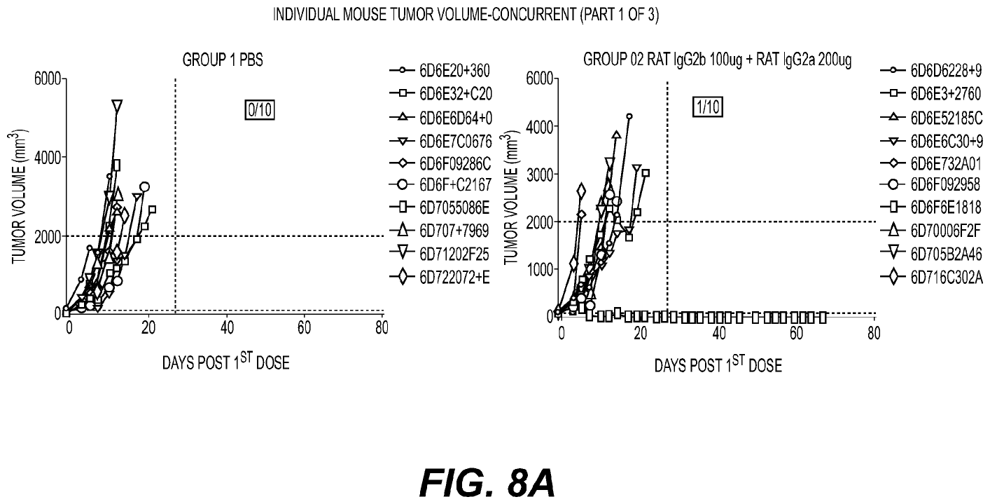

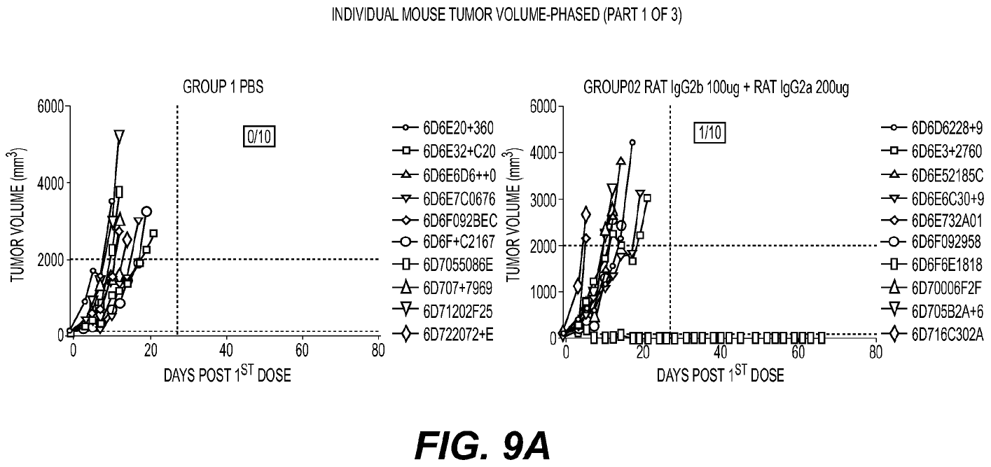

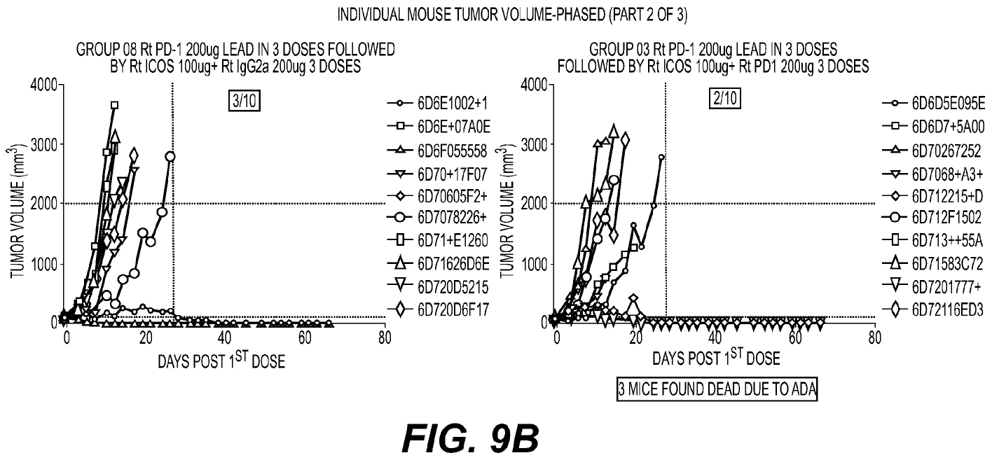

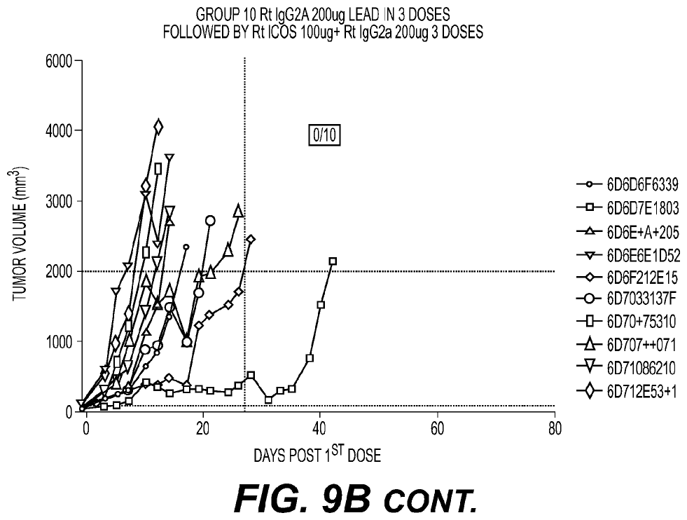

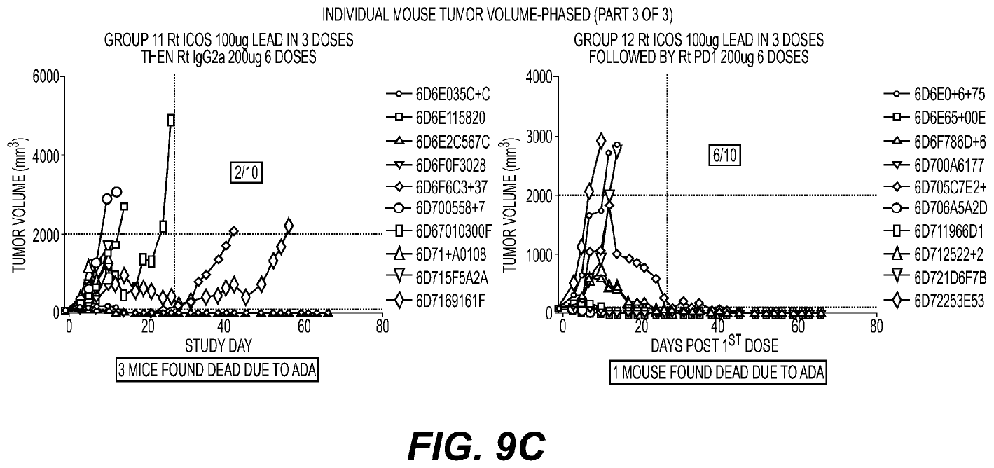

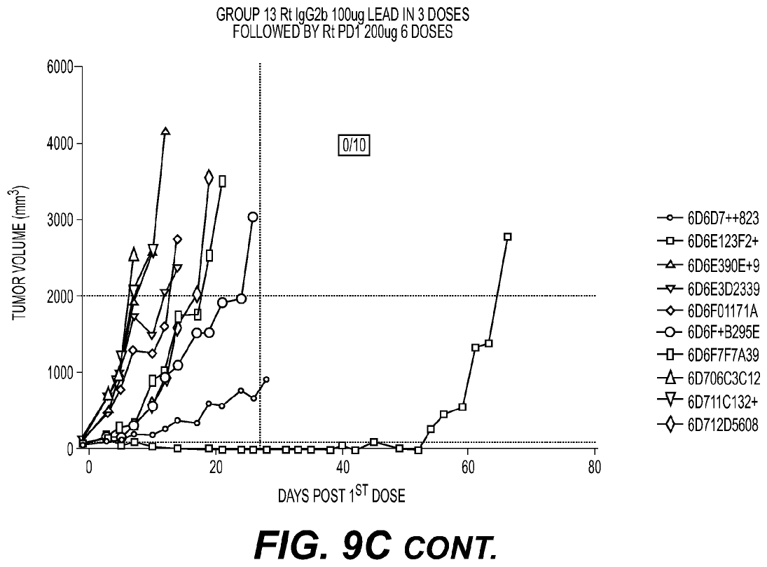

The study design of the anti-ICOS antibody/anti-PD1 antibody concurrent and phased dosing study conducted is shown in FIG. 1. FIG. 2 is a schematic showing the study procedure 5 of anti-ICOS antibody/anti-PD1 antibody concurrent and phased dosing study. Shown at the bottom of FIG. 2 is a table listing antibodies used in the study. In FIGS. 3-7, FIGS. 8A-8C, FIGS. 9A-9C, and FIGS. 10-14, “Rt ICOS” refers to “rat anti-ICOS antibody;” “Rt PD1” refers to “rat anti-PD1 antibody.” “Rt IgG2A” refers to “rat IgG2A;” “Rt IgG2B” refers to “rat IgG2B.”

### Monotherapy:

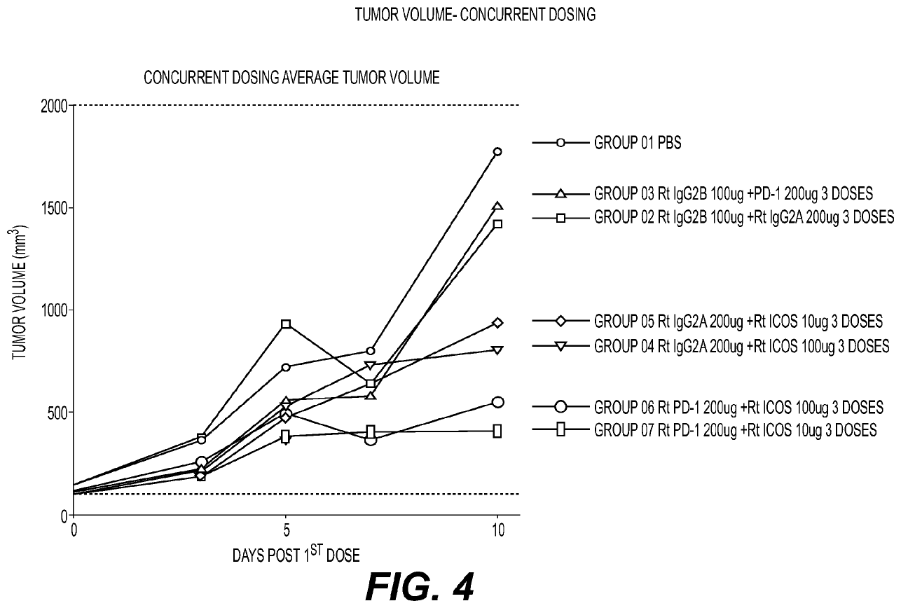

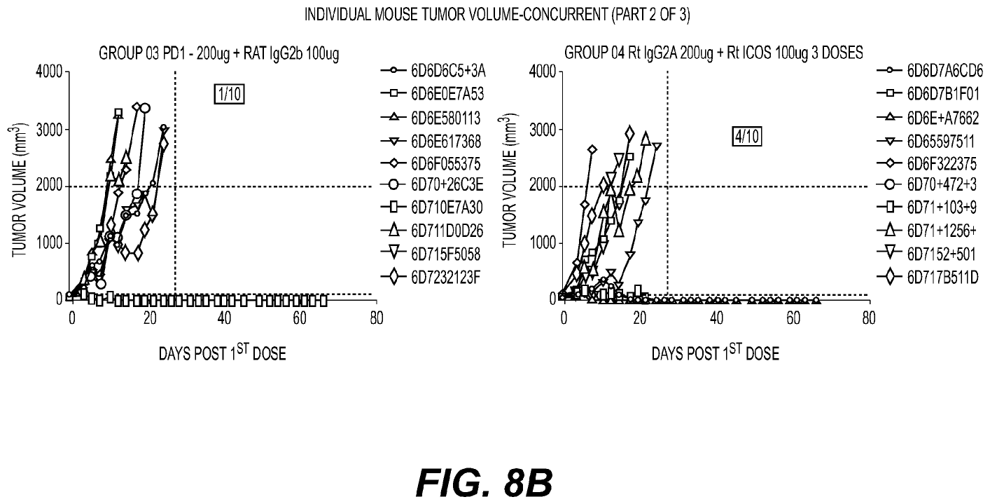

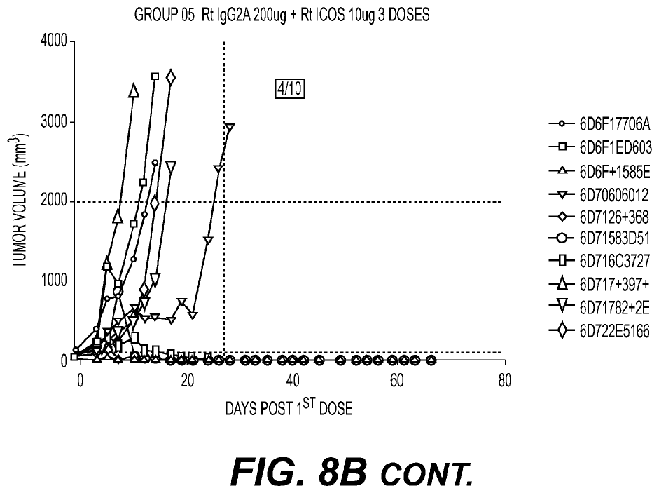

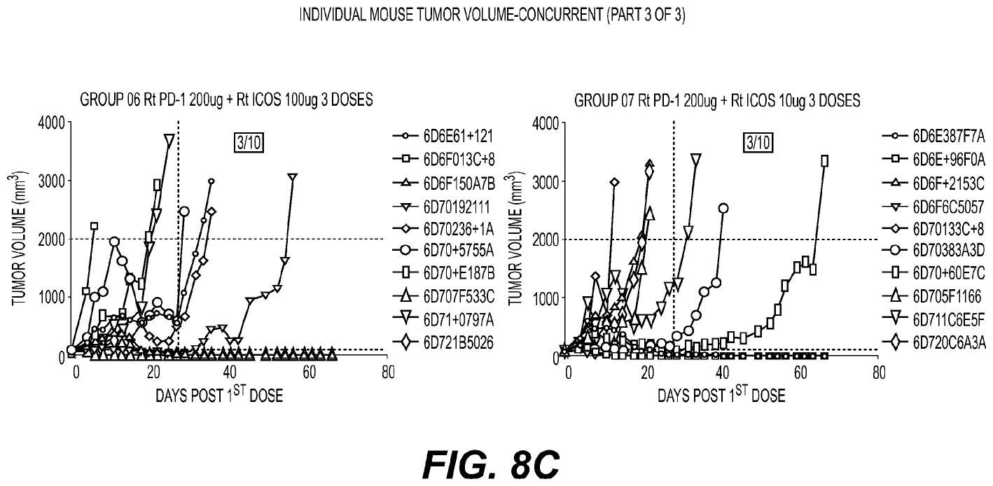

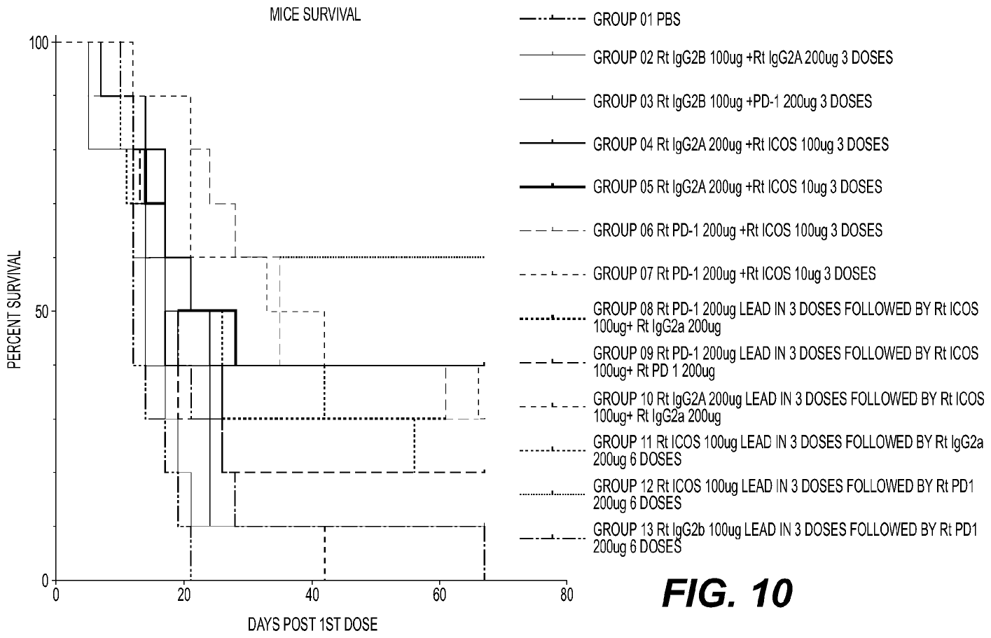

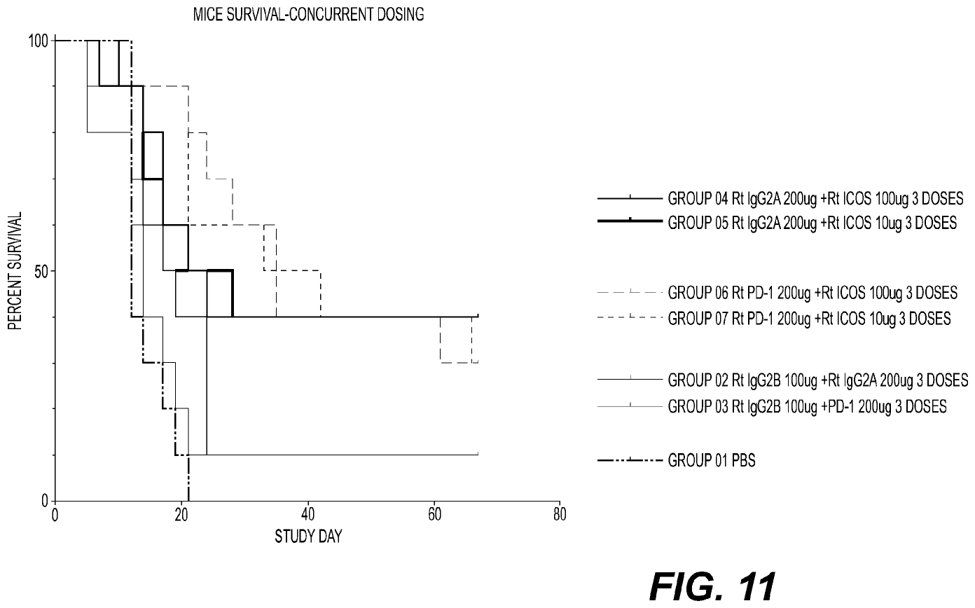

As shown in FIGS. 3, 4, 8B, 10 and 11, rat anti-mouse ICOS antibody (17G9) 100 μg or 10 μg showed similar tumor growth rate (FIG. 3, FIG. 4, FIG. 8B) and overall survival (40%) (FIG. 10, FIG. 11).

Rat anti-mouse anti-PD1 antibody (200 μg) had no effect on tumor growth rate (FIG. 3, FIG. 4, FIGS. 8A-8B). Overall survival was 10% (FIG. 10, FIG. 11).

### Combination:

At day 10, concurrent dosing of anti-ICOS antibody (100 μg or 10 μg) combined with anti-PD1 antibody showed synergistic anti-tumor efficacy compared to mono or phased dosing regimen (FIGS. 3-7, 8A-8C, 9A-9C).

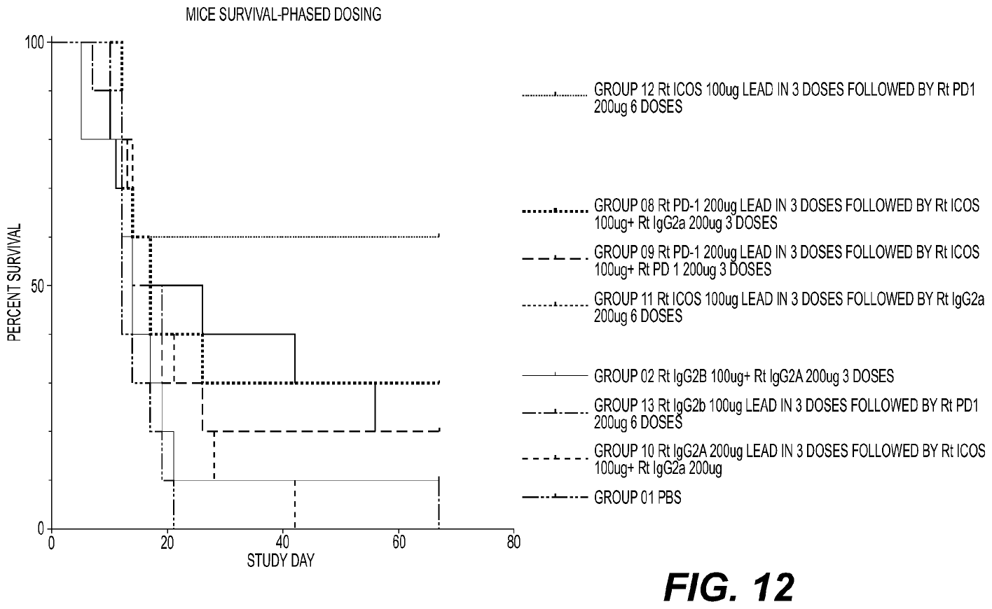

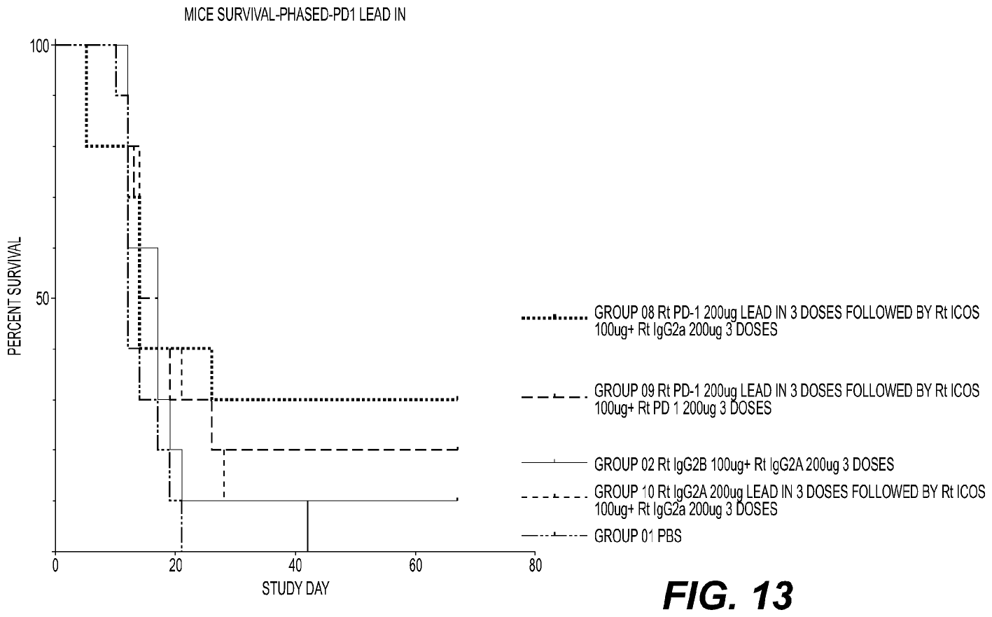

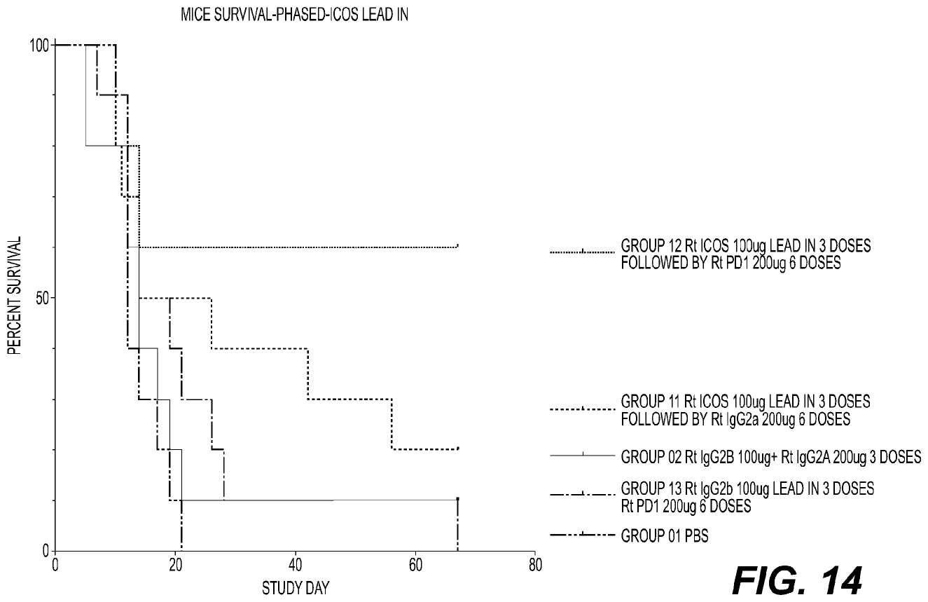

Mice in Group 12 treated with anti-ICOS lead-in/anti-PD1 follow up dosing showed surprising and unexpected increase in long term survival. Regarding mouse long term survival (day 67 post 1^{st} dose), 60% of mice from Group 12 (anti-ICOS lead in followed by 6 doses of anti-PD1) showed complete response (6 mice were tumor free, 1 mouse found dead due to anti-drug antibodies (ADA)) (FIG. 10, FIG. 12, FIG. 14). Twenty percent (20%) of mice from Group 11 (anti-ICOS lead in followed by 6 doses of rat IgG2A) showed complete response (3 mice were tumor free, 3 mice were found dead due to ADA) (FIG. 10, FIG. 12, FIG. 14); the data is comparable to the anti-ICOS monotherapy data. Thirty percent (30%) of mice from Group 8 (anti-PD1 lead in followed by 3 doses of anti-PD1+ rat IgG2b) showed complete response (3 mice are tumor free) (FIG. 10, FIG. 12, FIG. 13); this showed better overall survival than 3 doses of anti-PD1 (10%, 1 tumor free mouse). Twenty percent (20%) of mice from Group 9 (anti-PD1 lead in followed by 3 doses of anti-PD1+ anti-ICOS) showed complete response (3 mice were tumor free, 3 mice were found dead due to ADA) (FIG. 10, FIG. 12, FIG. 13). ADA occurred at the 4^{th} and 5^{th} doses.

The results described herein in Example 1 were obtained with the following materials and methods.

### Mice, Tumor Challenge and Treatment

All studies were conducted in accordance with the GSK Policy on the Care, Welfare and Treatment of Laboratory Animals and were reviewed by the Institutional Animal Care and Use Committee (IACUC) either at GSK or by the ethical review process at the institution where the work was performed. 6-8 week old female BALB/c mice (Envigo) were utilized for in vivo studies in a fully accredited AAALAC facility.

5.0×10⁴ cells/mouse of CT26 mouse colon carcinoma (ATCC CRL-2638) tumor cells were inoculated subcutaneously into the right flank. Tumor volume and body weight data were collected using the Study Director™ software package (Studylog Systems, South San Francisco, Calif., USA). Tumor volume was calculated using the formula: Tumor Volume (mm)³=0.52*1*w² where w=width and l=length, in mm, of the tumor. When tumors reached approximately 50-100 mm³, mice were randomized into various groups (n=10/treatment group) based on tumor volume using stratified sampling method in the Study Log™ software prior to initiation of treatment. Tumor measurement of greater than 2,000 mm³ for an individual mouse and/or development of open ulcerations in tumor and/or body weight loss greater than 20% resulted in mice being removed from study. Dosing started on randomization day. Mice received the mouse anti-ICOS (clone 7E.17G9) and/or mouse anti-PD1 (clone RMP1-14) antibodies or saline via intraperitoneal injection twice weekly starting on randomization day for a total of 3 doses of anti-ICOS and 3 or 6 doses of anti-PD1 for concurrent and sequential dosing respectively. In order to evaluate anti-tumor activity of combining the anti-ICOS and anti-PD-1 monoclonal antibodies, mice were dosed twice a week with either anti-ICOS (clone 7E.17G9, rat IgG2b 100 μg) or its isotype control (rat IgG2b 100 μg) along with anti-PD-1 (clone RMP1-14, rat IgG2a 200 μg) or its isotype control (rat IgG2a 200 μg) concurrently. For the experiments involving sequential dosing, either dosing with anti-ICOS antibody started after 3 doses of anti-PD1 which meant that the the last 3 of the 6 anti-PD1 doses were given in combination with anti-ICOS or anti-PD1 dosing started after all 3 doses of the anti-ICOS antibody were completed. Appropriate isotype controls were also employed in a similar dosing regimen.

Data are plotted using Graphpad™ software and Statistical analysis was performed by Statistician.

### Example 2

Characterization of an IgG4 Anti-ICOS Agonist Antibody that Elicits T-Cell Activation and Antitumor Responses Alone and with PD-1 Blockade

Described in Example 2 is the characterization of the immune-stimulatory and anti-tumor activity of a humanized non-depleting anti-ICOS agonist antibody, with an emphasis on the importance of isotype choice for optimal efficacy and provides strong rationale for exploring this in cancer patients as a single agent and in combination with PD-1 checkpoint blockade.

Inducible T-cell Co-Stimulator (ICOS) is a T-cell-restricted co-stimulatory receptor whose expression is induced on activated T cells upon T-cell receptor engagement. We demonstrate that antibody-mediated ICOS agonism elicits potent T-cell activation, mobilization of T cells to the tumor site, and antitumor responses in syngeneic mouse models. Our data indicate that the isotype choice for the agonist antibody is crucial to avoid Fc-dependent cytotoxicity and depletion of effector T cells (T_{eff}), as observed with an IgG1 version of the antibody tested. Furthermore, our data suggest that ICOS expression level on regulatory T cells (T_{reg}), albeit high, offers a narrow window for selective depletion of T_{regs} in most tumors, due to overlapping ICOS levels on T_{eff} and the upregulation of ICOS in the presence of checkpoint blockade. Exploration of isotypes led to the selection of a humanized IgG4 anti-ICOS agonist antibody (H2L5 IgG4PE) for clinical development. We present the characterization of the immunological activity and therapeutic potential of this ICOS agonist antibody, currently being investigated alone and in combination with pembrolizumab in a first-in-human clinical study.

### INTRODUCTION

Inducible T-cell co-stimulator (ICOS) is a co-stimulatory receptor with structural and functional homology to the CD28/CTLA-4-Ig superfamily (Hutloff, A. Nature 397:263-266 (1999)). ICOS expression is upregulated by antigen stimulation and ICOS signaling induces production of both T_{H}1 and T_{H}2 cytokines and effector T-cell (T_{eff}) proliferation. ICOS expression has been observed on resting T_{H}17, T follicular helper (T_{FH}) and regulatory T (T_{reg}) cells; however, unlike CD28, it is not highly expressed on most resting naïve and memory T-cell populations (Fazilleau, N. et al. Nat Immunol. 8(7):753-61. (2007), Paulos, C. M. et al. Sci Transl Med. 2(55): 55-78. (2010)). ICOS plays a crucial role in the survival and expansion of T_{eff} and T_{reg} during an immune response (Burmeister, Y. et al. J Immunol. 180(2): 774-82. (2008)) and has been shown to be critical for the development and function of T_{H}17 (Paulos, C. M. et al. Sci Transl Med. 2(55): 55-78. (2010), Guedan, S. et al. Blood 124(7): 1070-80. (2014)).

Emerging data from patients treated with anti-CTLA-4 antibodies suggest ICOS-expressing memory T cells may help mediate antitumor immune responses and long-term survival (Liakou, C. I. et al. Proc Natl Acad Sci USA. 105(39): 14987-92. (2008); Di Giacomo, A. M. et al. Cancer Immunol Immunother. 62(6): 1021-8. (2013); Carthon, B. C. et al. Clin Cancer Res. 16(10): 2861-71. (2010); Vonderheide, R. H. et al. Clin Cancer Res. 16(13): 3485-94. (2010)). ICOS has been shown to be critical for anti-CTLA-4 antitumor activity in mice (Fu, T. He, Q., Sharma, P. Cancer Res. 71(16): 5445-54. (2011); Fan, X, et al. J Exp Med. 211(4):715-25. (2014)) and prior reports support the concept that activating ICOS on CD4 and CD8 T cells using recombinant murine ICOS ligand has antitumor potential (Ara, G. et al. Int. J Cancer. 103(4): 501-7 (2003)). Human ICOS ligand (ICOS-L) has been shown to bind both CTLA-4 and CD28 in addition to ICOS, which limits the potential use of recombinant ICOS-L as a therapeutic in humans (Yao, S. et al. Immunity 34(5), 729-40. (2011)); necessitating an alternative therapeutic approach to activate ICOS in patients with cancer.

Here, we describe the immunologic and antitumor characterization of a first-in-class humanized IgG4 anti-ICOS agonist monoclonal antibody (mAb) H2L5 IgG4PE, designed to deliver optimal ICOS agonism via Fc gamma receptor (FcγR) cross-linking, with minimal antibody-dependent cellular cytotoxicity (ADCC) and phagocytosis activity; thereby reducing the risk of T_{eff} depletion. The comprehensive preclinical data described herein, support clinical testing of H2L5 IgG4PE, currently being investigated alone and in combination with pembrolizumab in a first-in-human clinical study.

### Results

Development of a Potent and Selective Anti-Human ICOS Agonist Monoclonal Antibody (mAb)

We undertook the generation of an agonistic anti-human ICOS mAb by immunizing mice with ICOS extracellular domain. One of these mAb was humanized and expressed as a human IgG4 with 2 Fc mutations (glutamic acid for leucine at residue 235) (Kabat, E. A., et al. Sequences of Proteins of Immunological Interest, 5th Ed. U.S. Dept. of Health and Human Services, Bethesda, Md., NIH Publication no. 91-3242. (1991)) and substitution of proline for serine at residue 228 (EU numbering) to reduce antigen binding fragment (Fab) arm exchange with native IgG4 (Manjula, P. et al. The Journal of Immunology. 164:1925-1933. (2000), Rispens, T. et al. J. Am. Chem. Soc. 133 (26):10302-10311. (2011)). The resulting H2L5 IgG4PE hereafter referred to as “H2L5”.

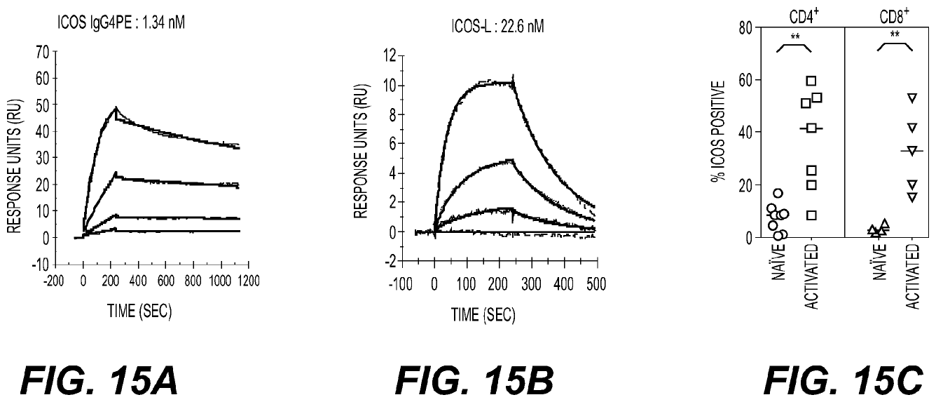

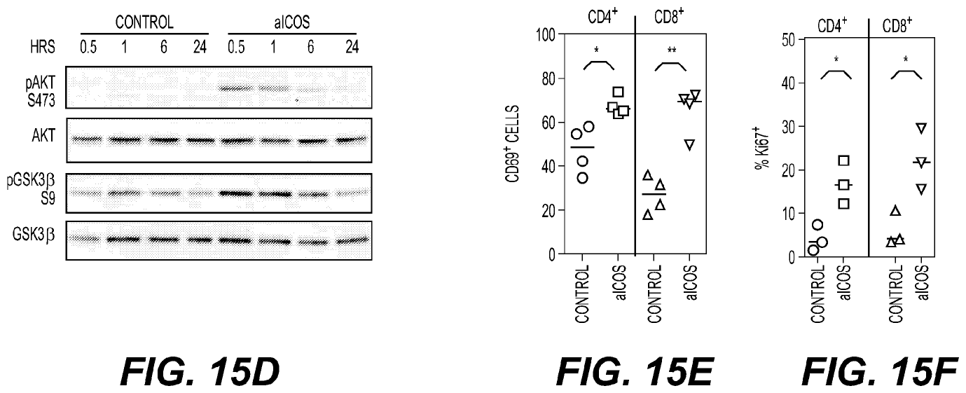

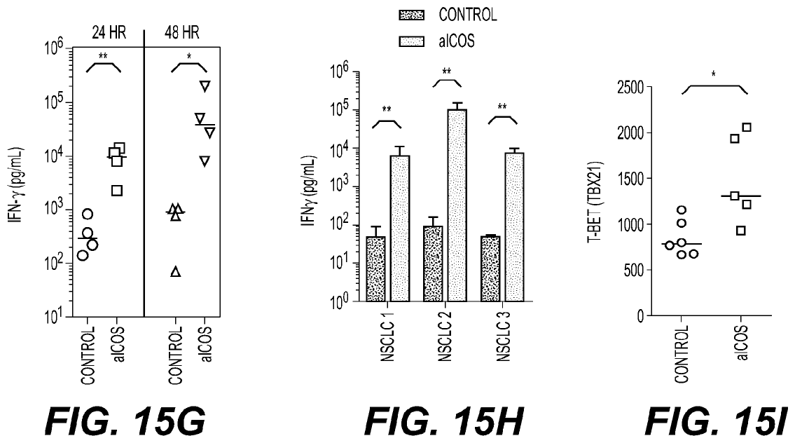

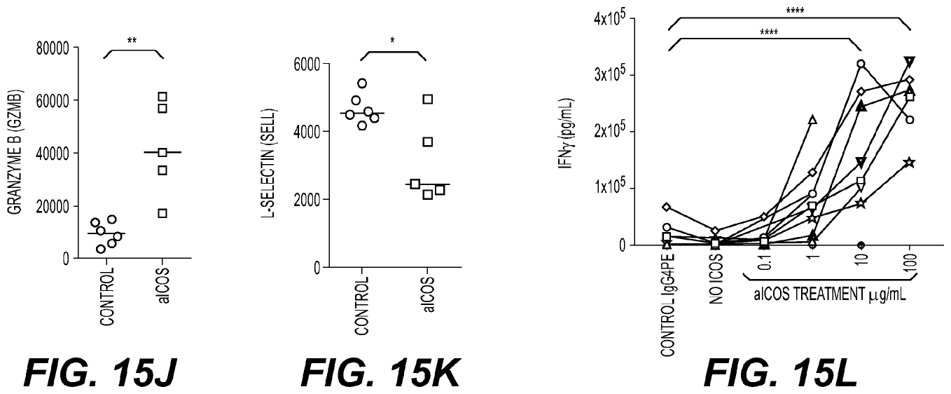

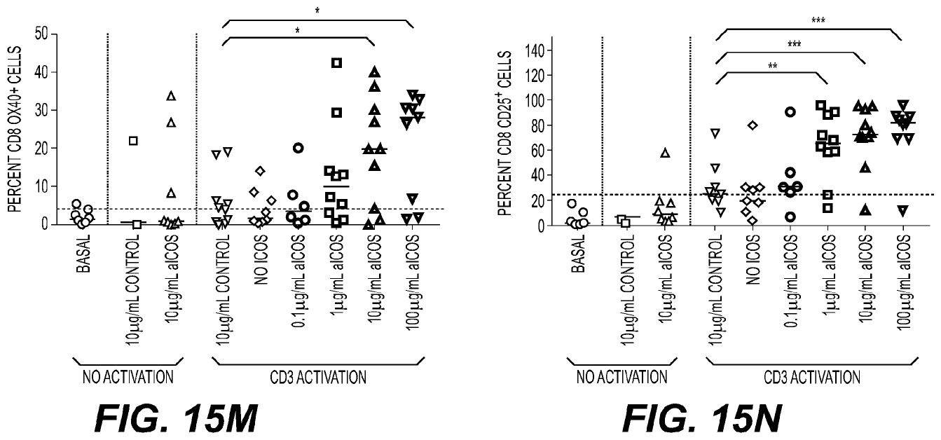

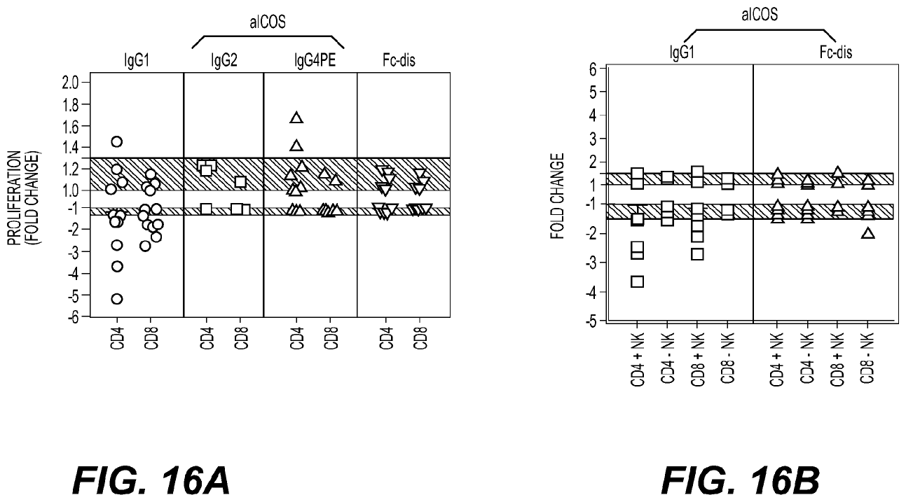

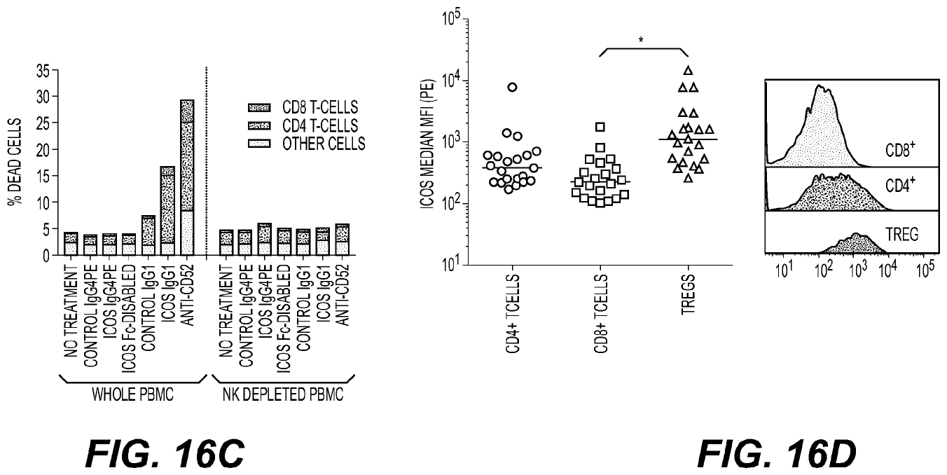

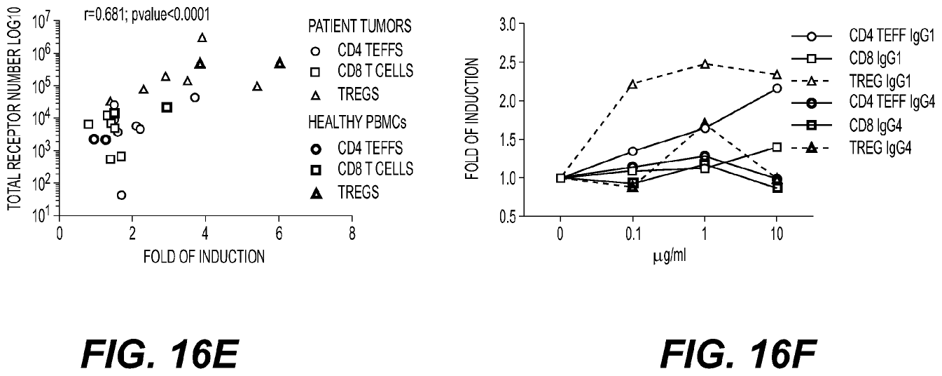

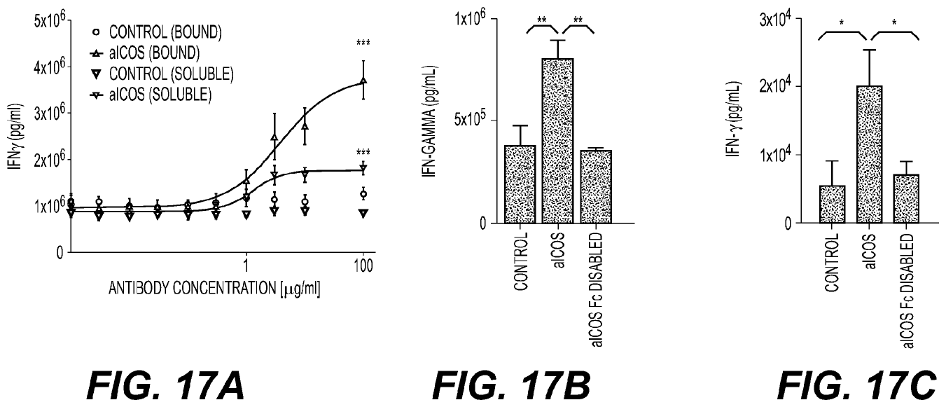

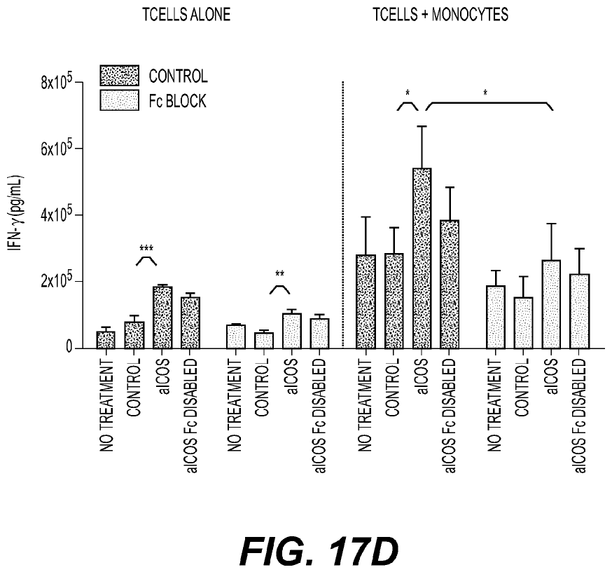

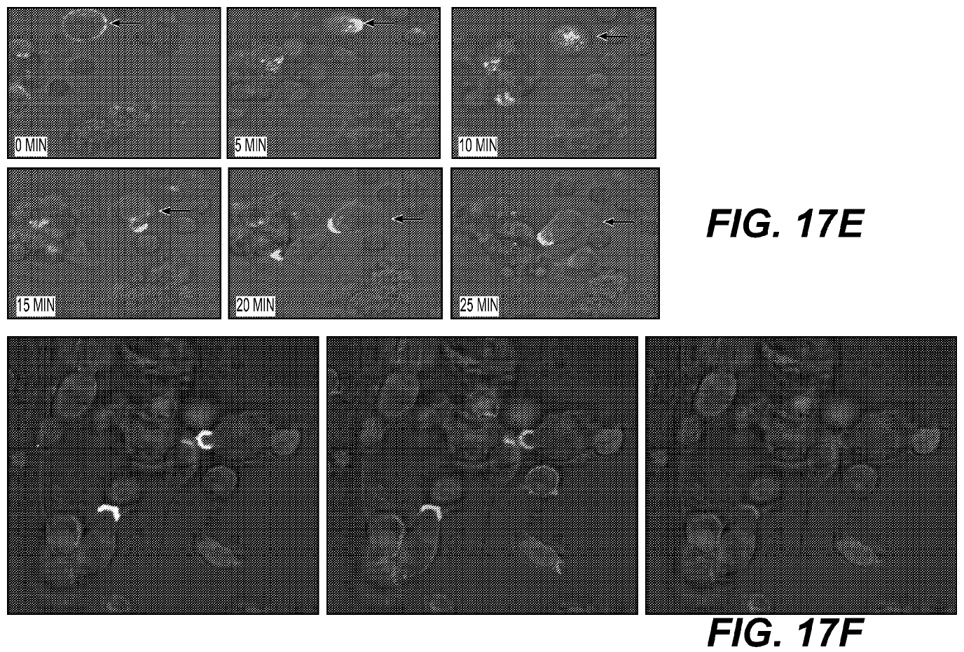

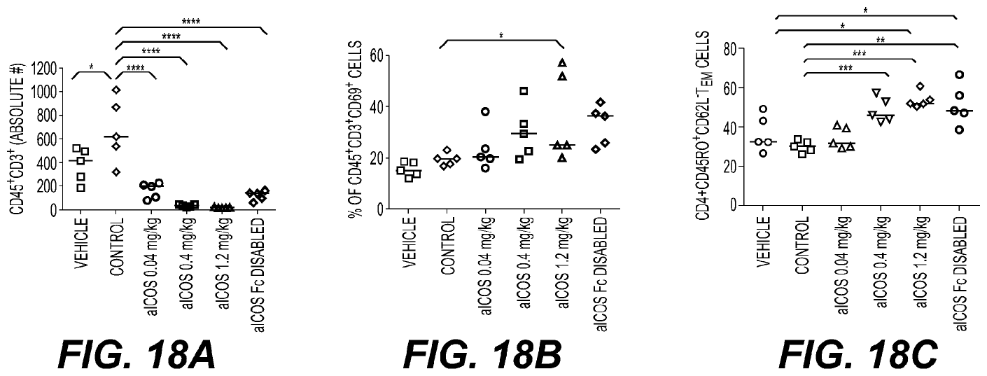

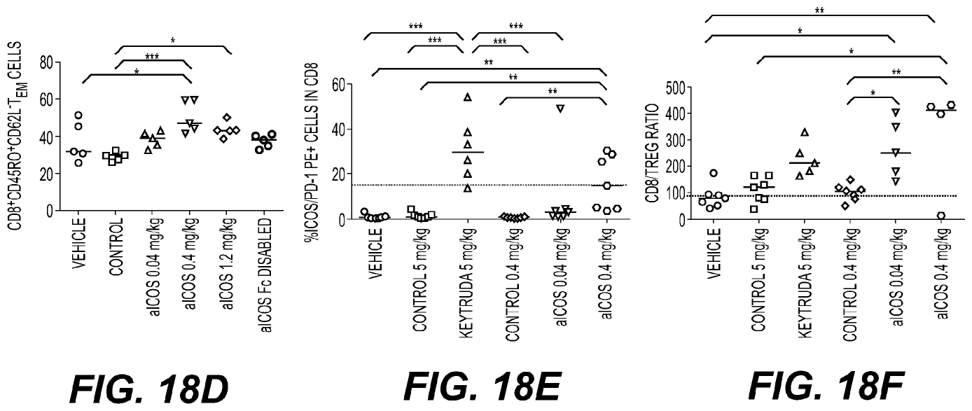

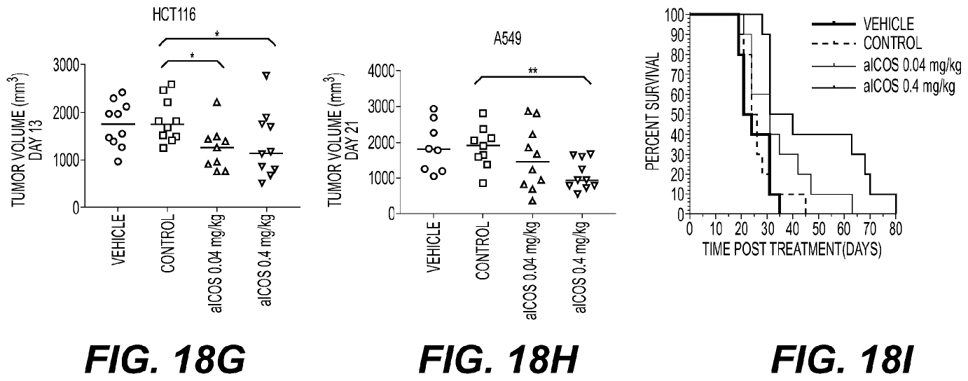

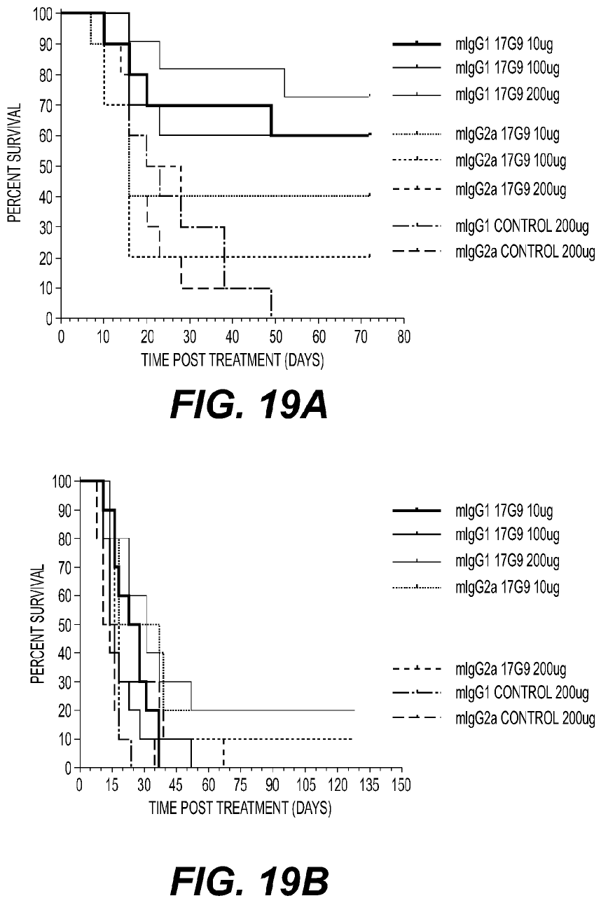

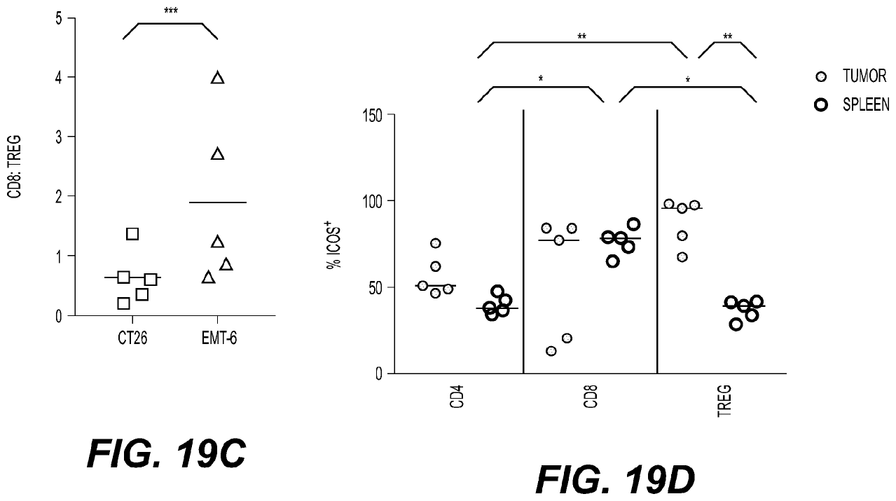

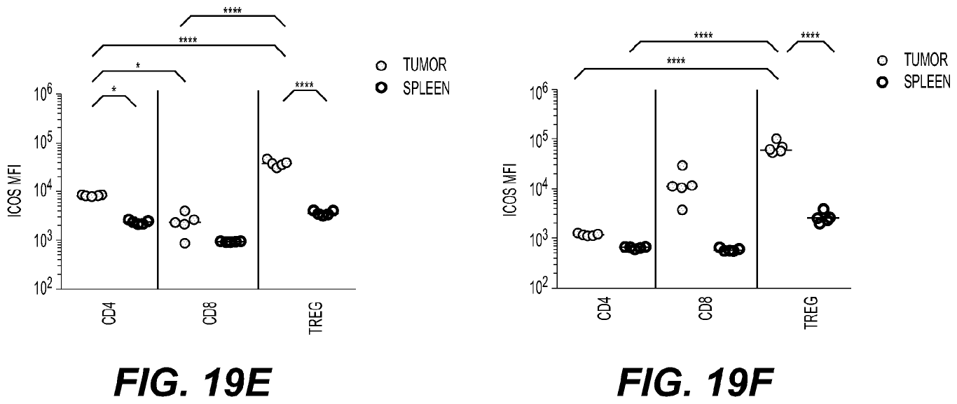

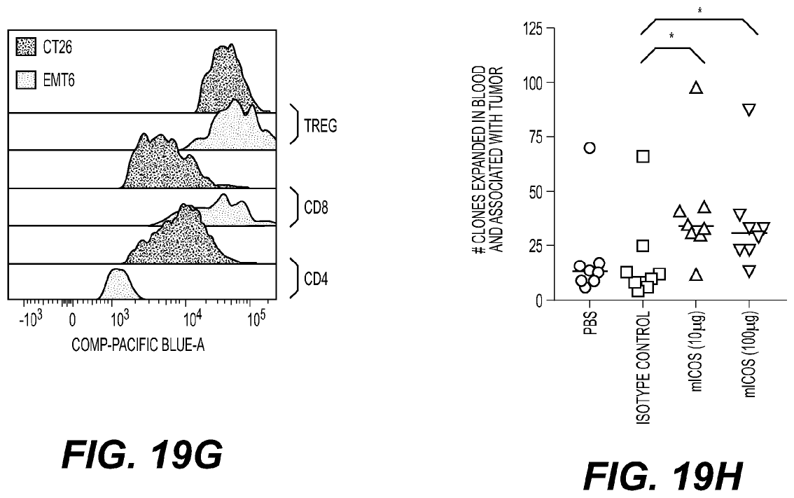

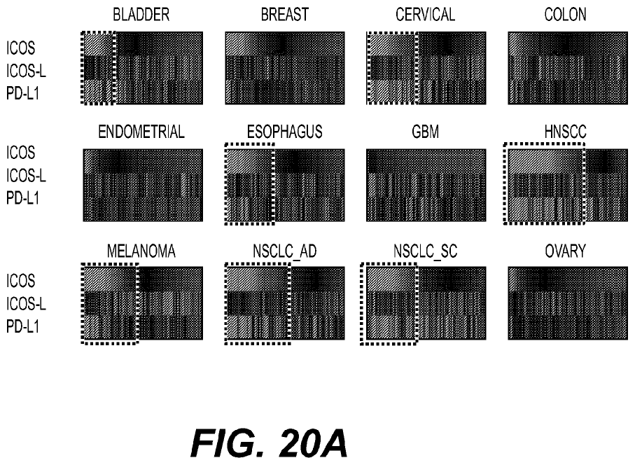

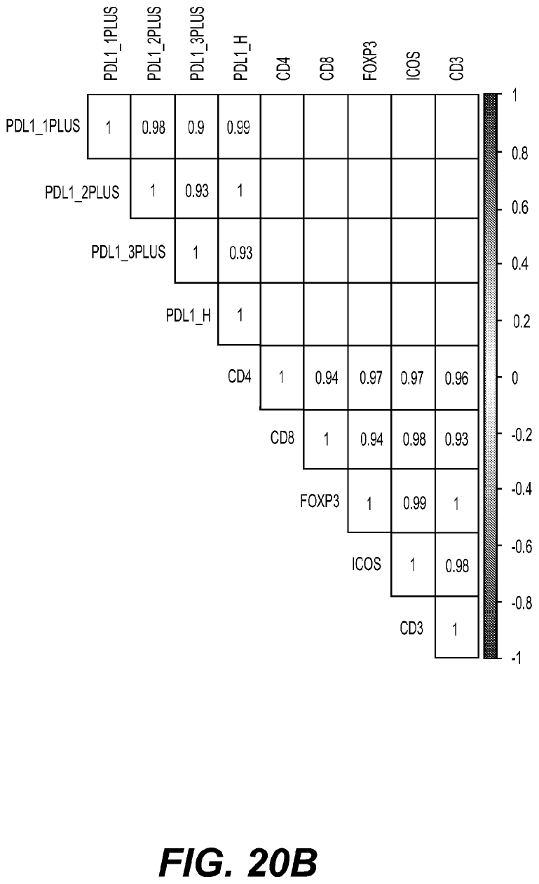

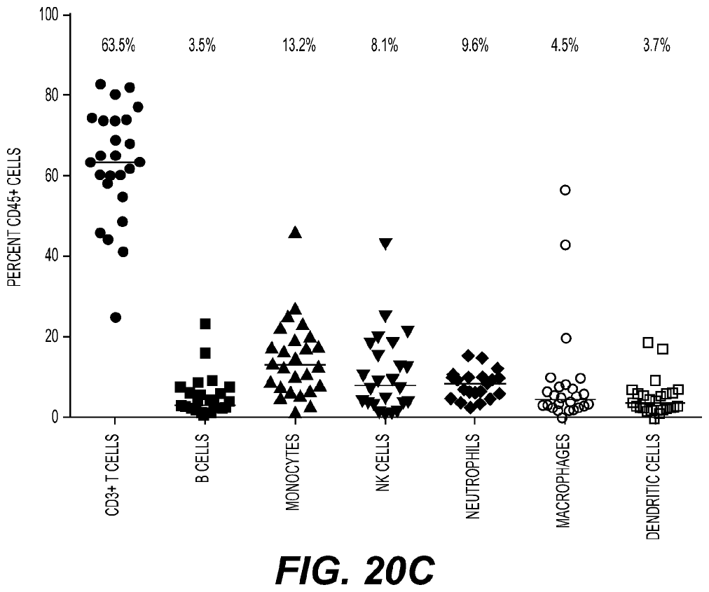

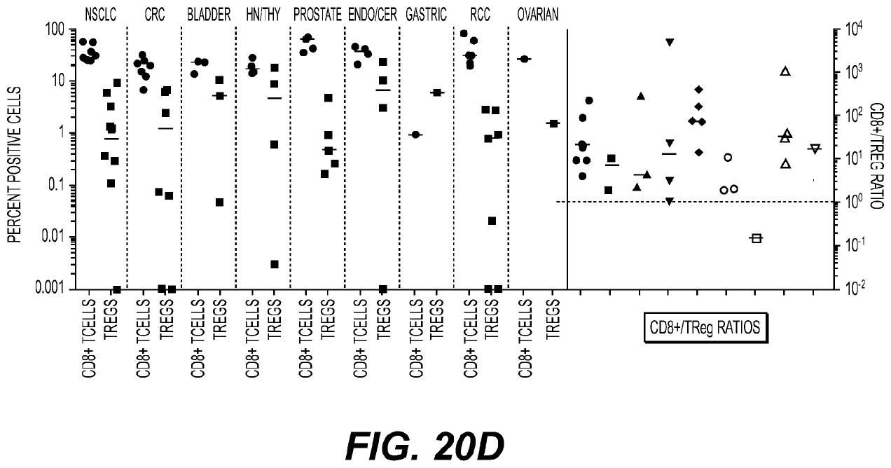

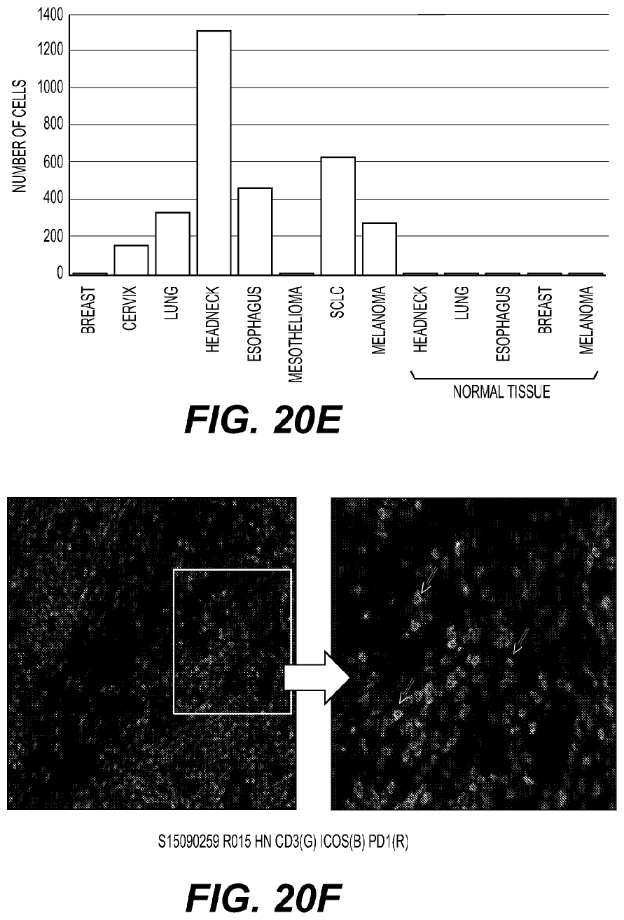

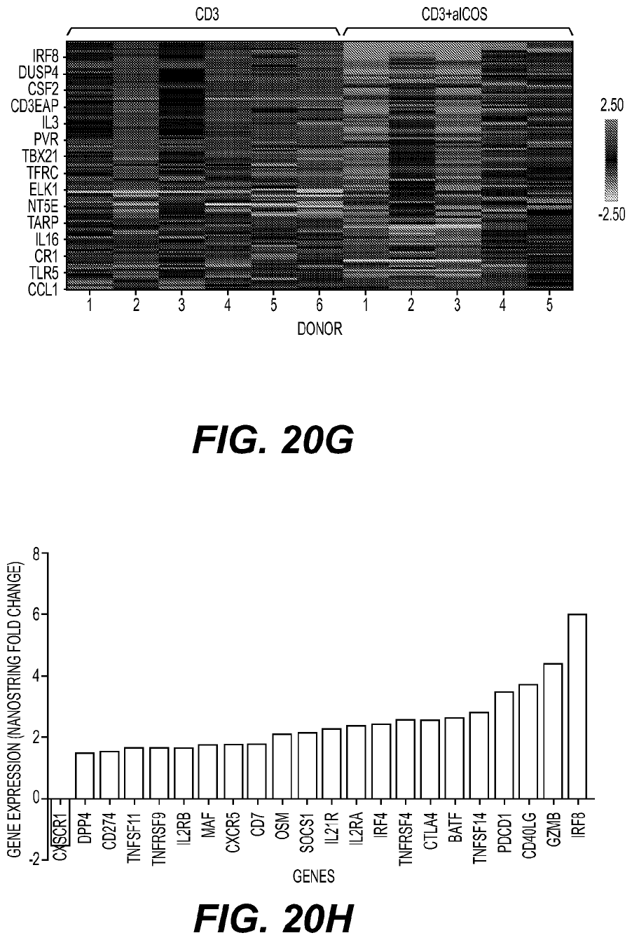

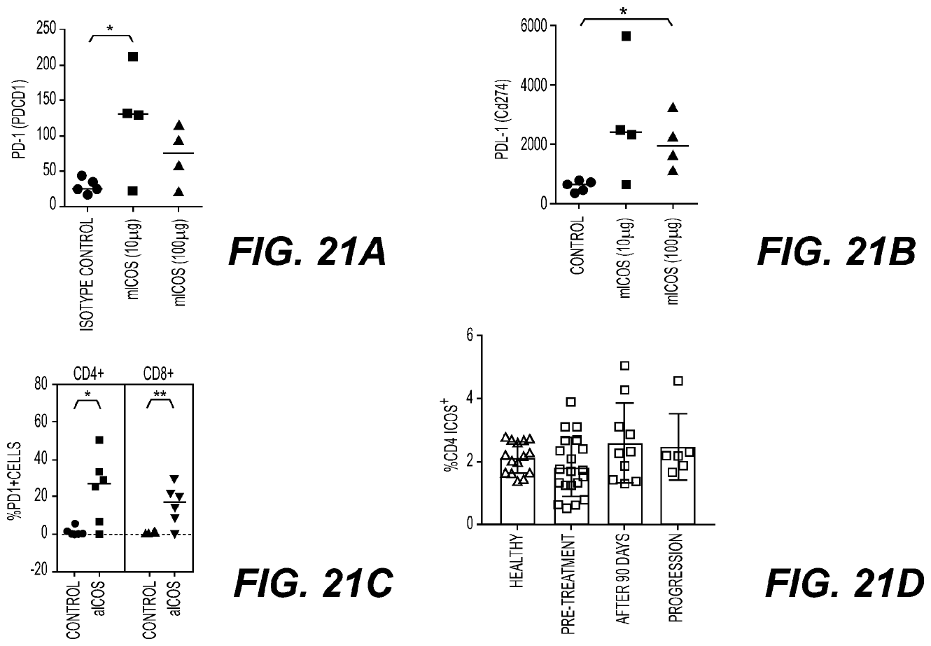

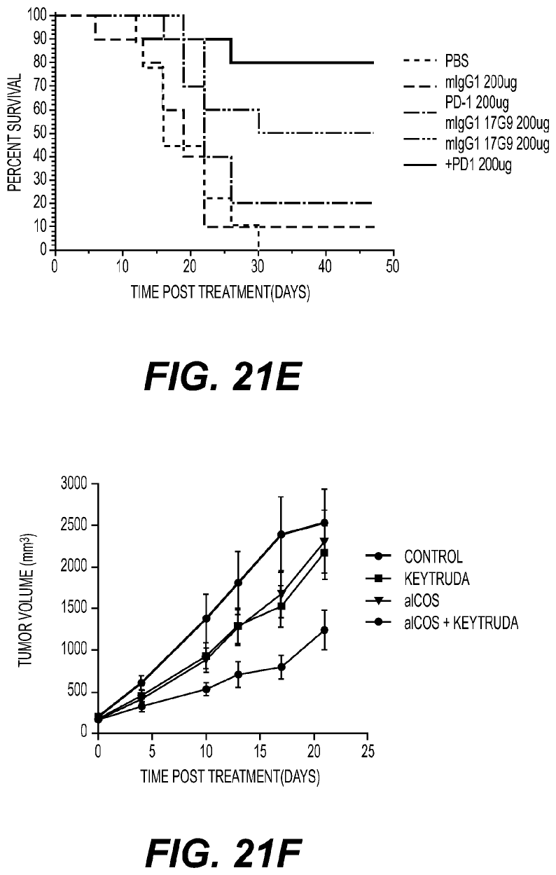

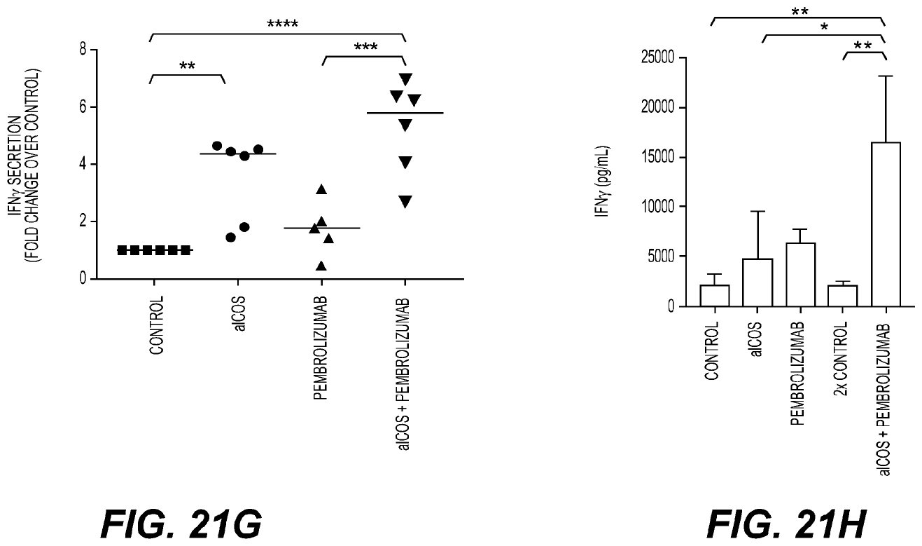

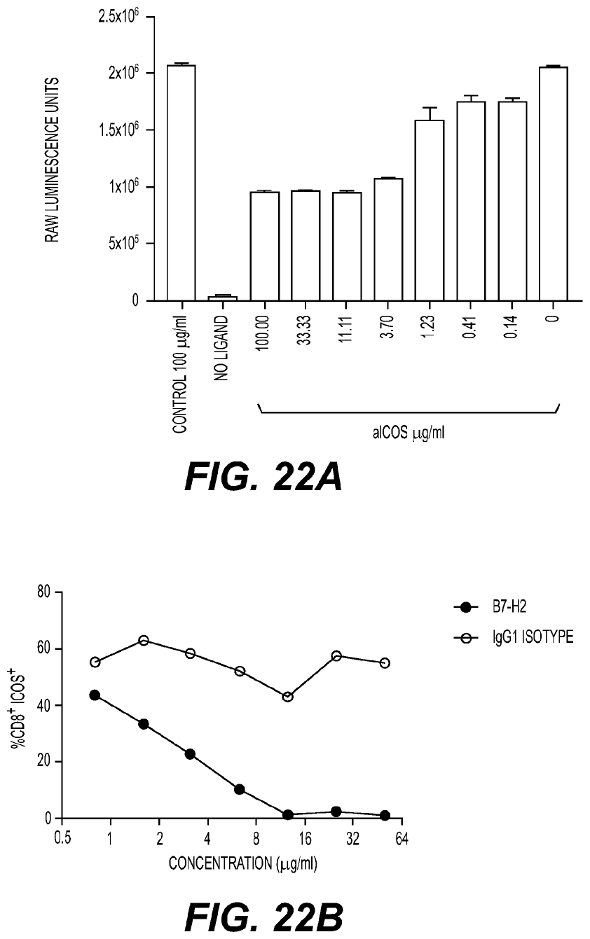

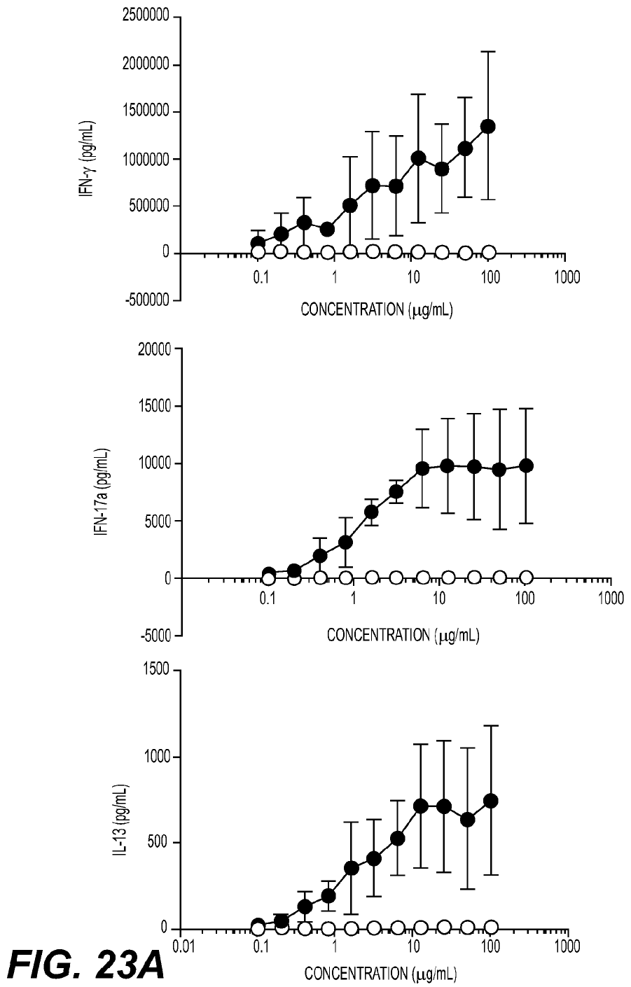

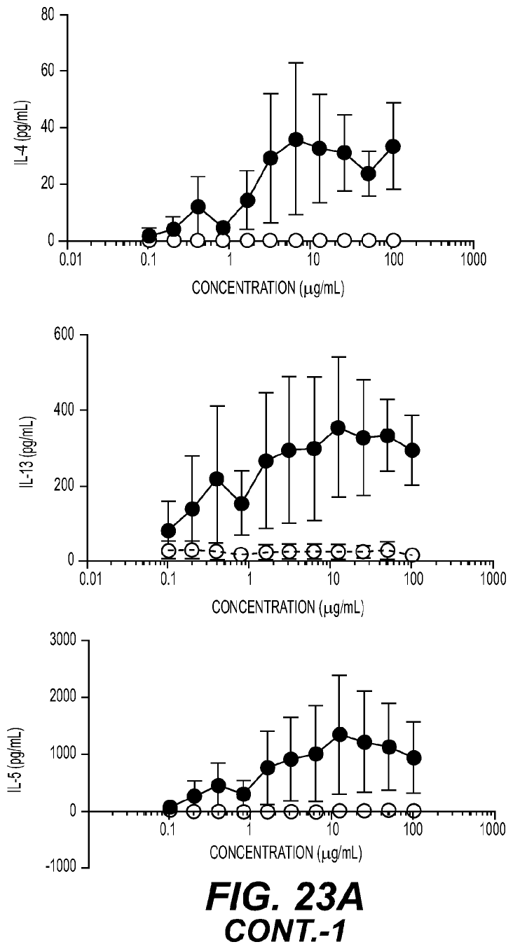

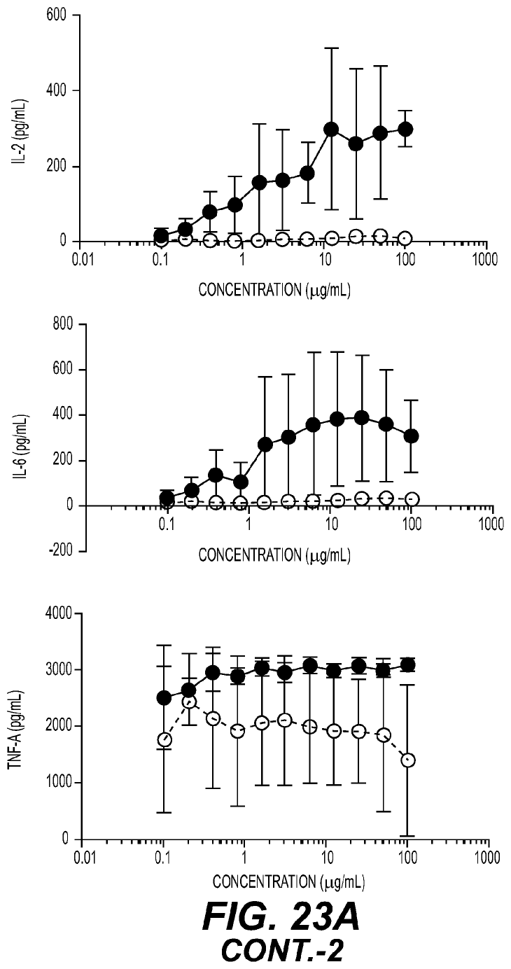

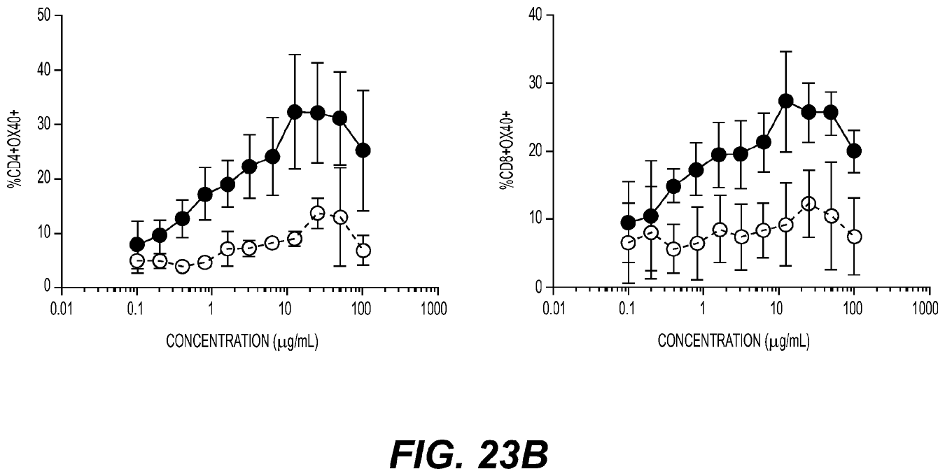

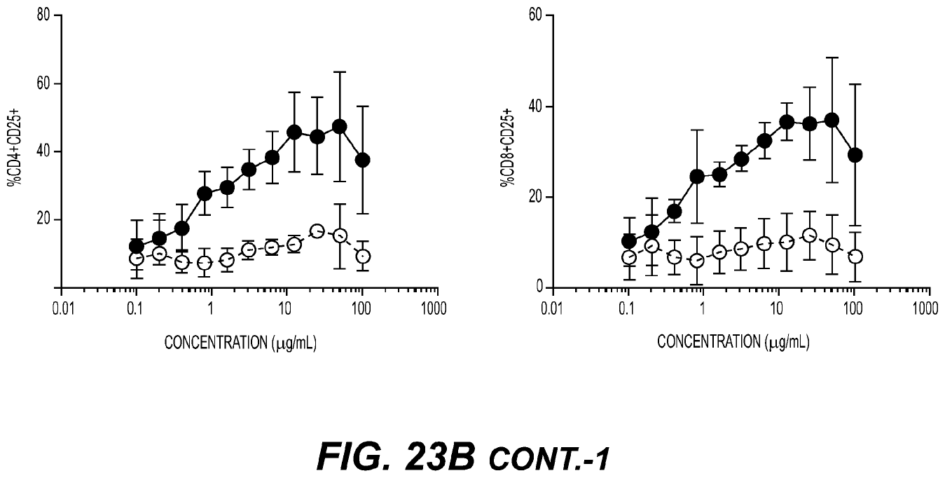

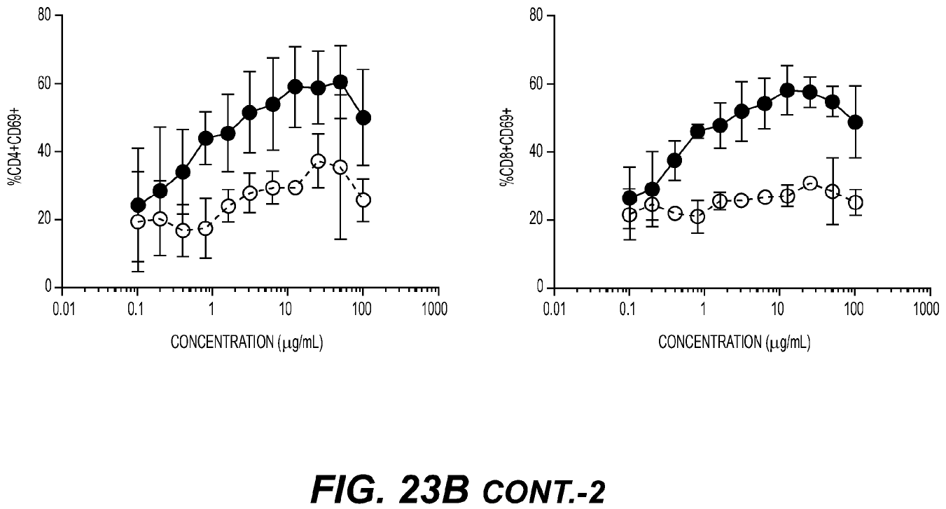

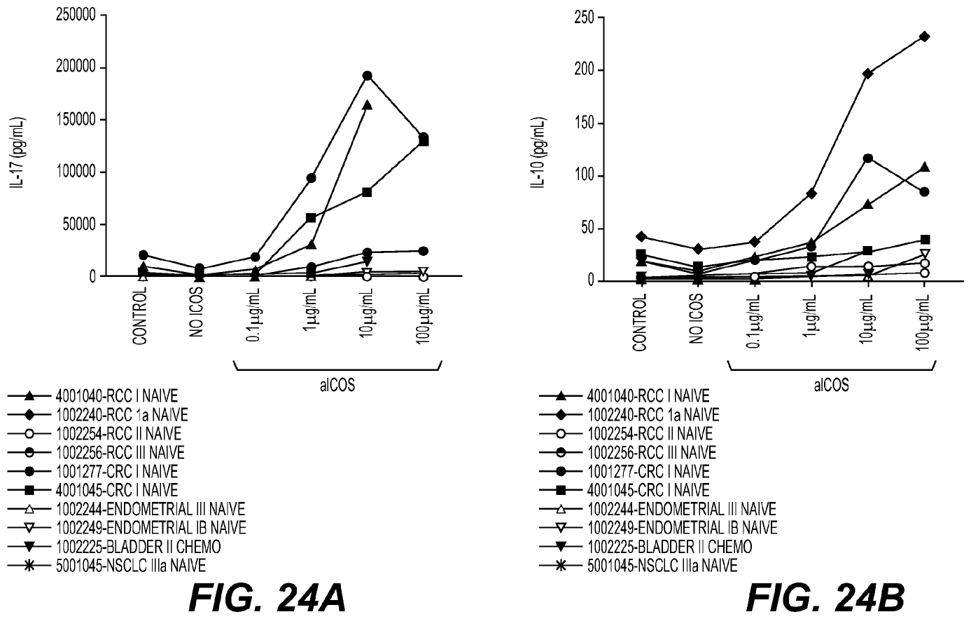

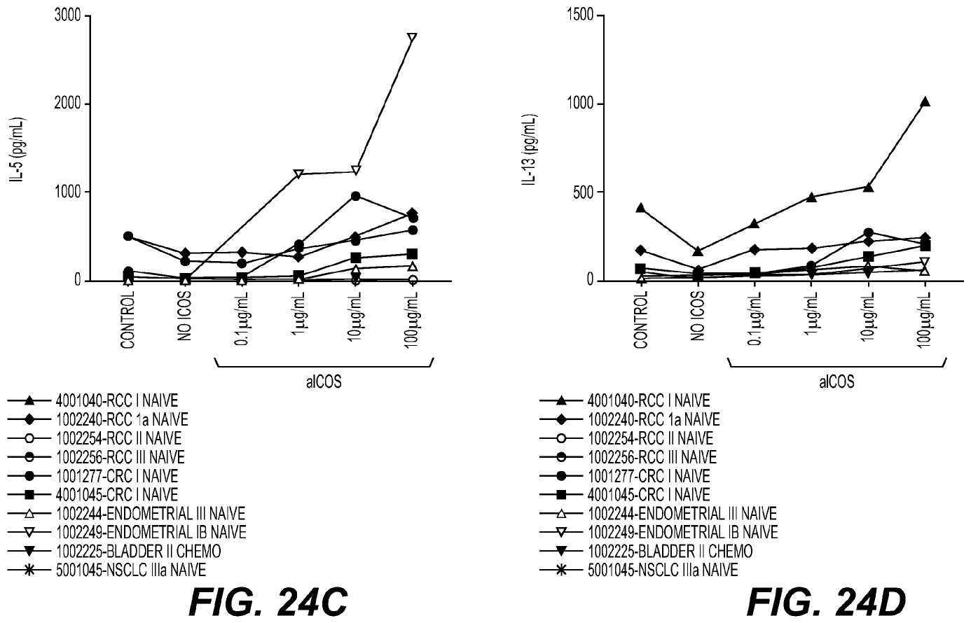

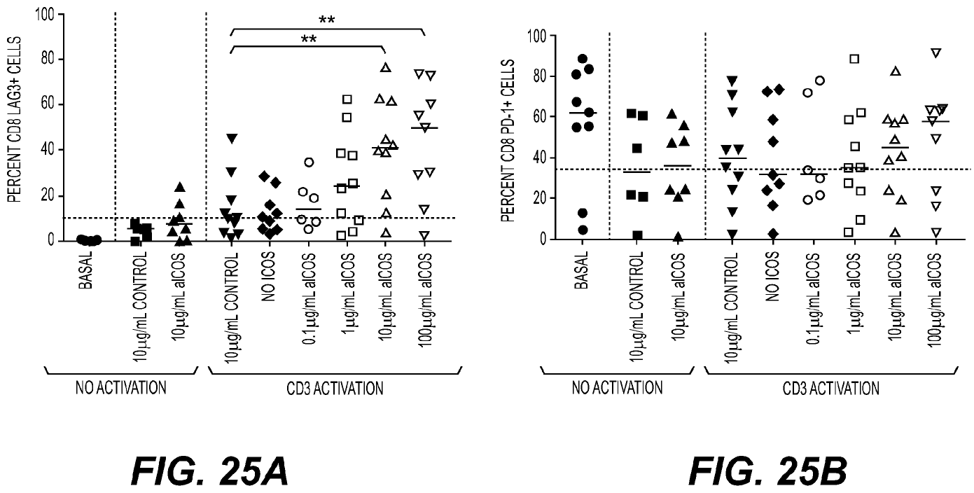

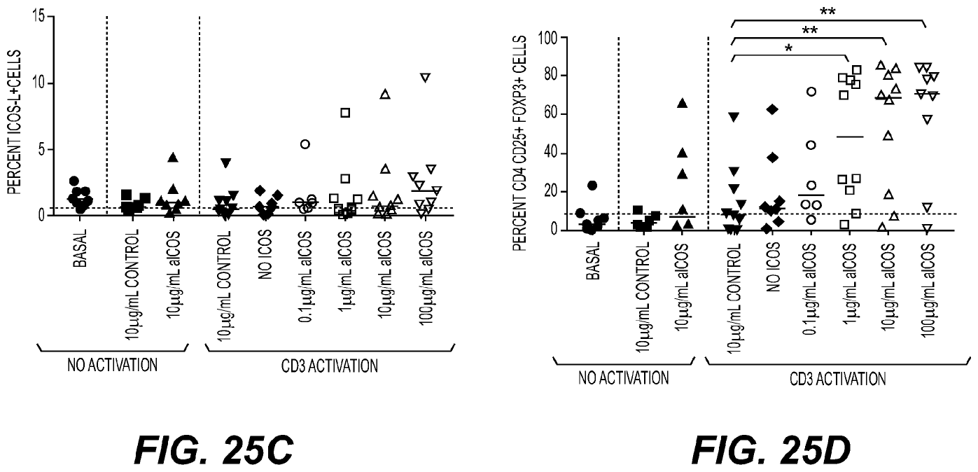

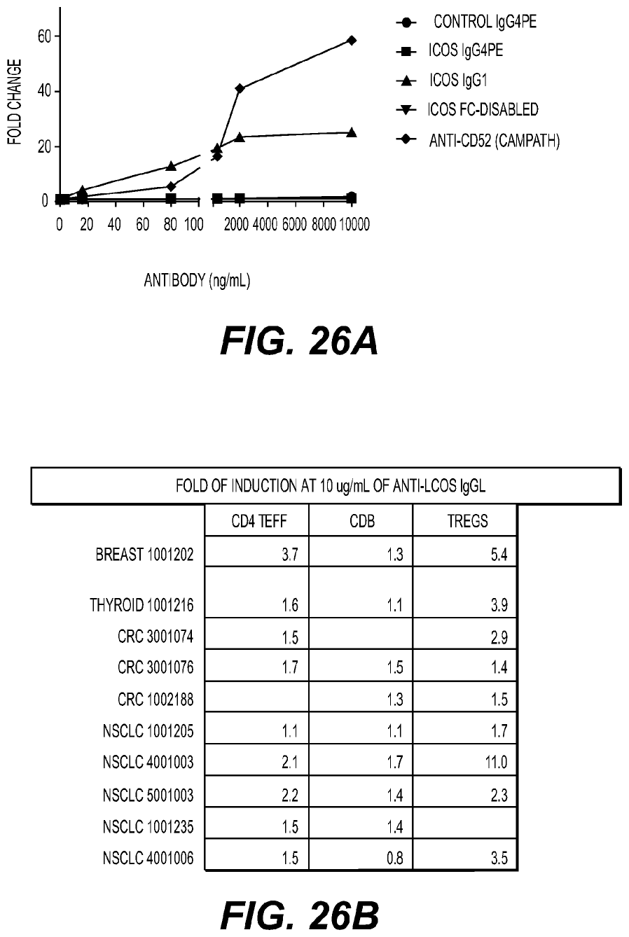

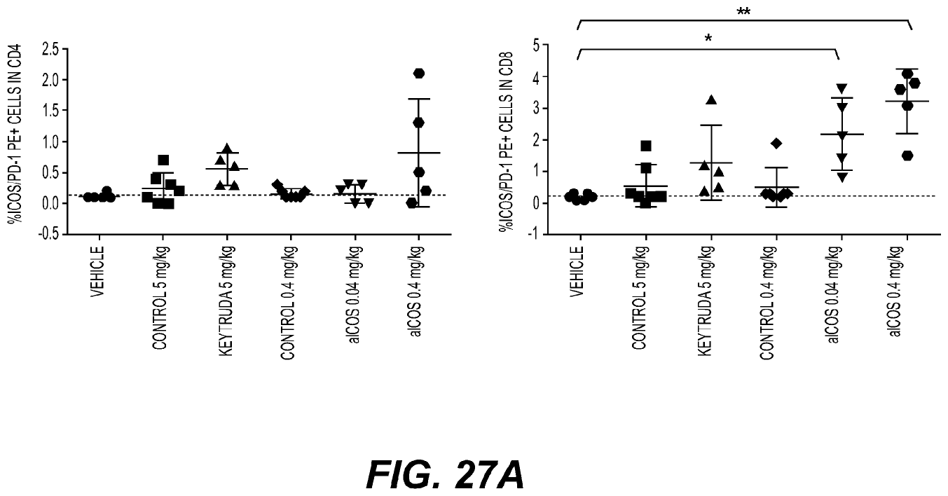

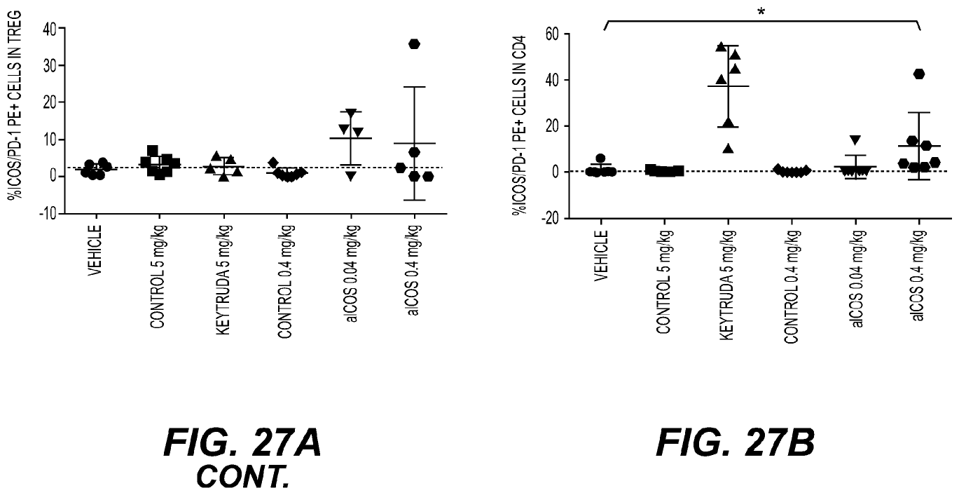

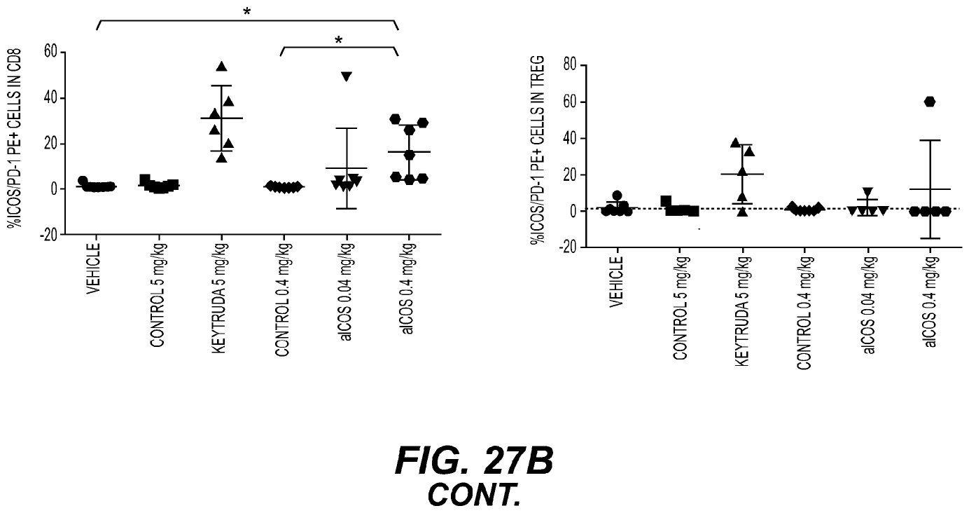

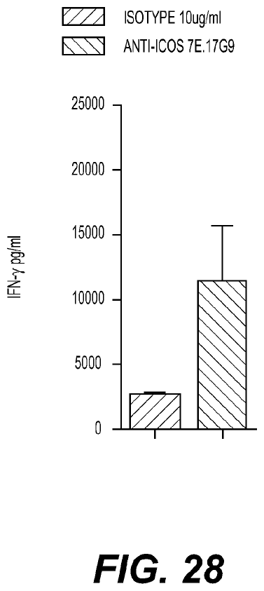

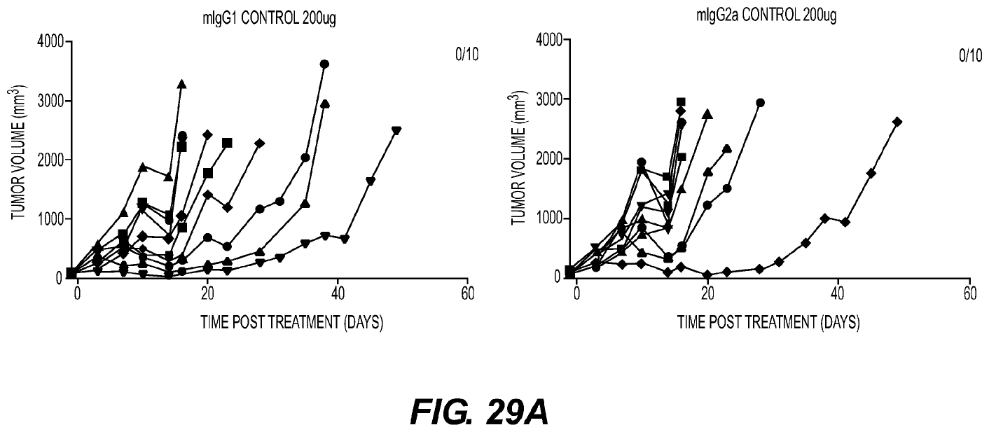

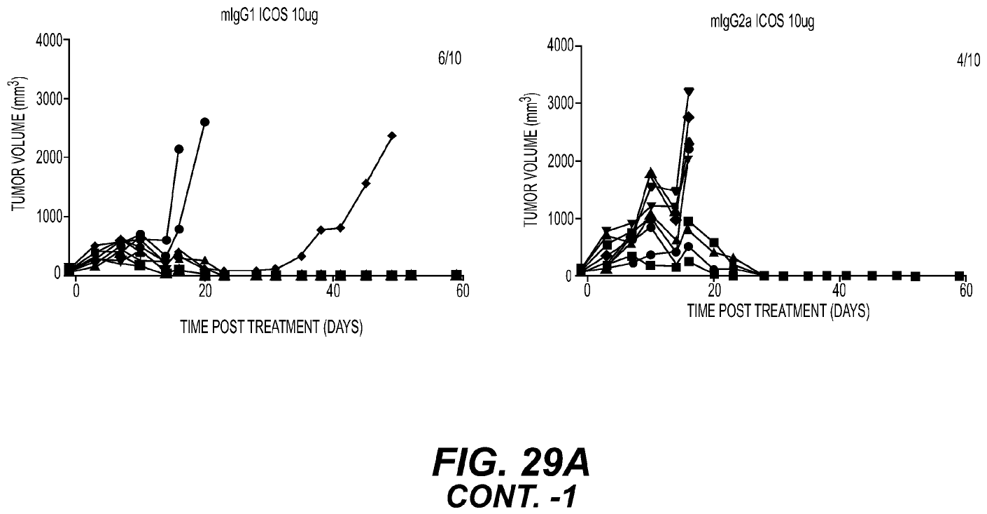

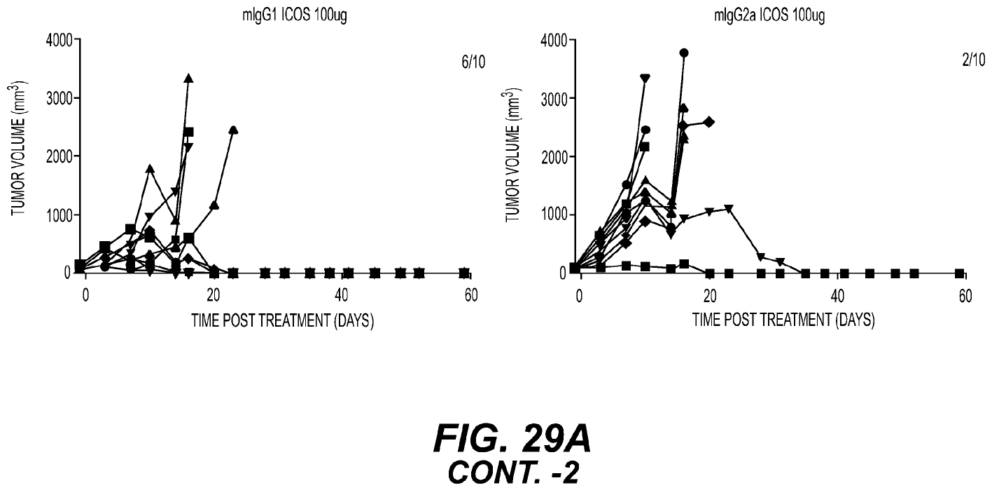

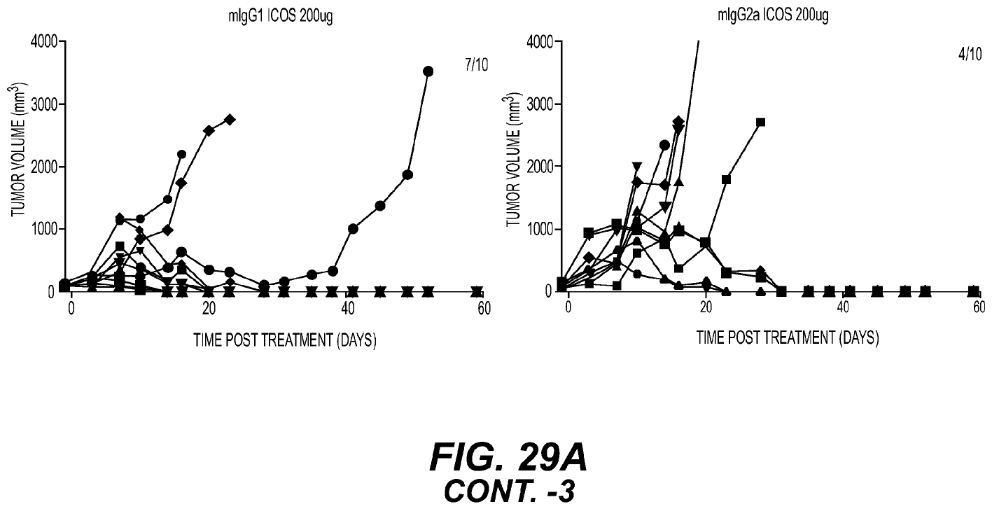

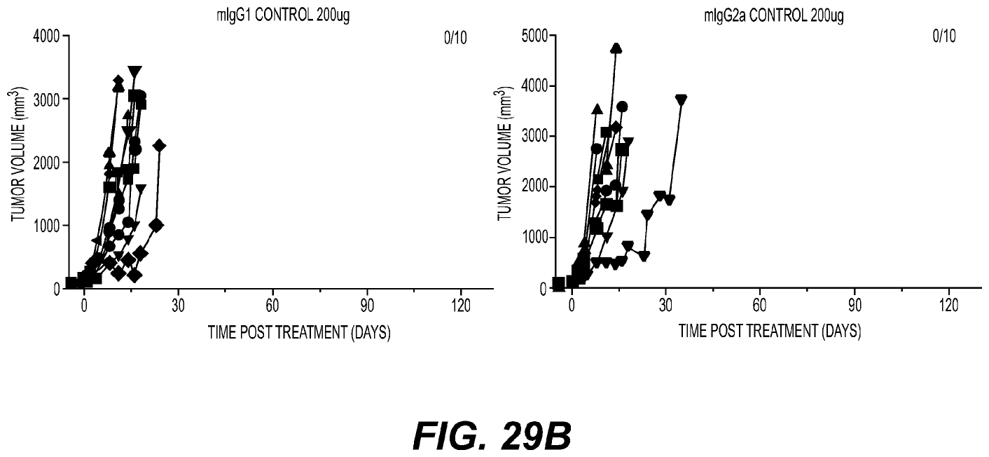

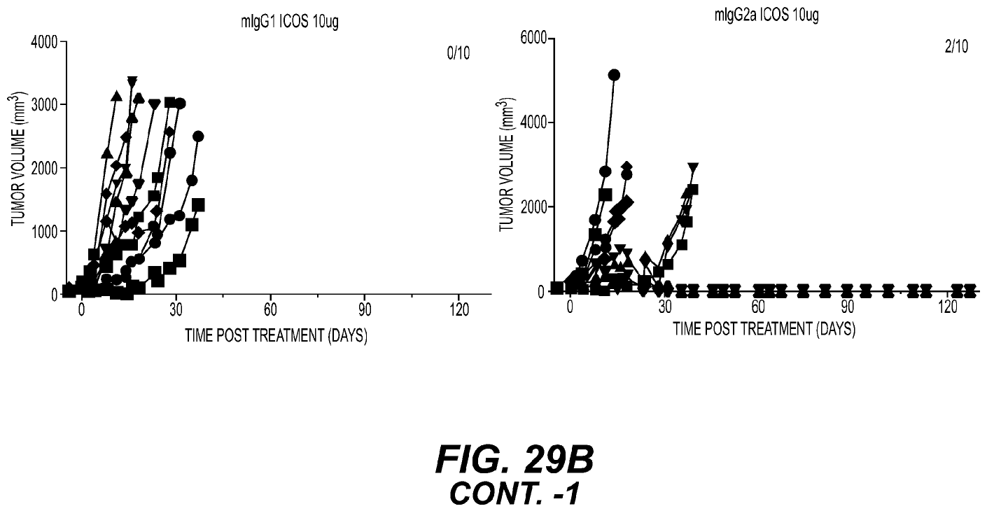

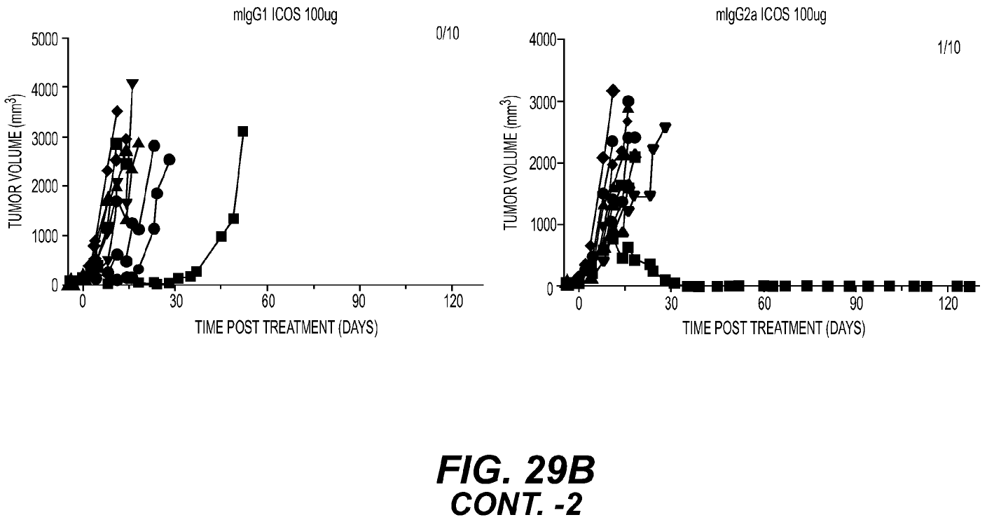

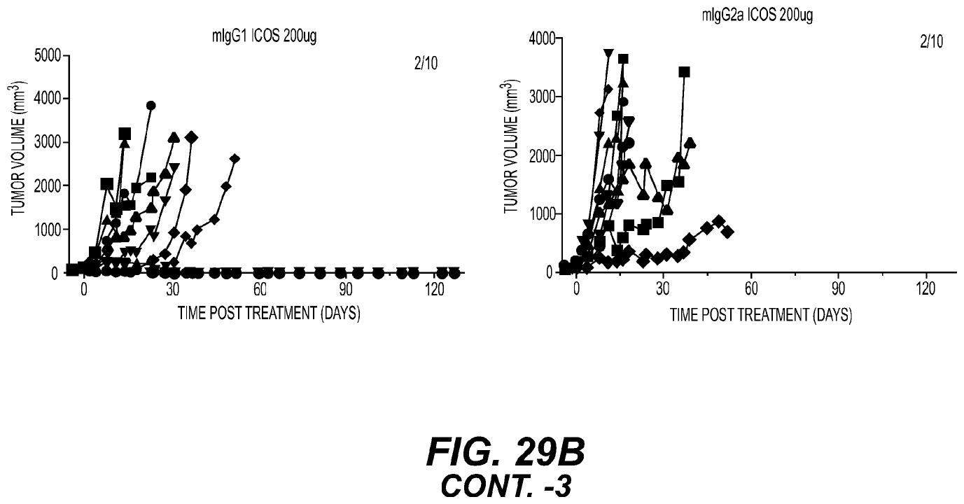

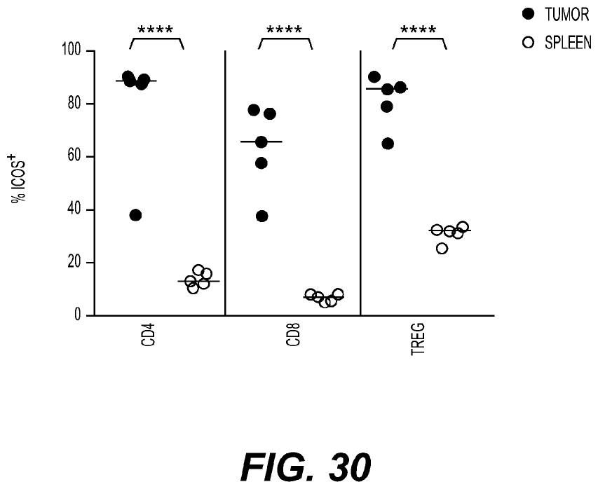

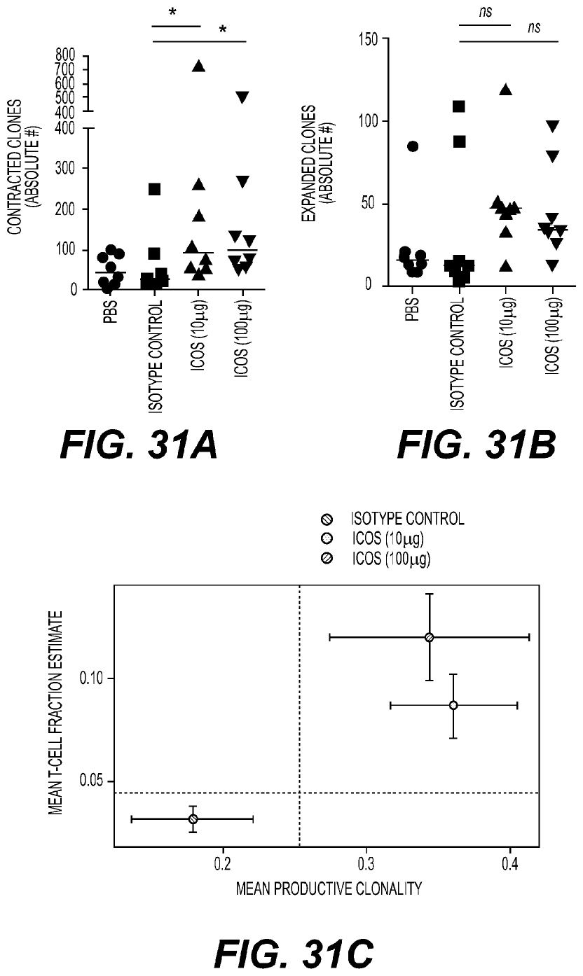

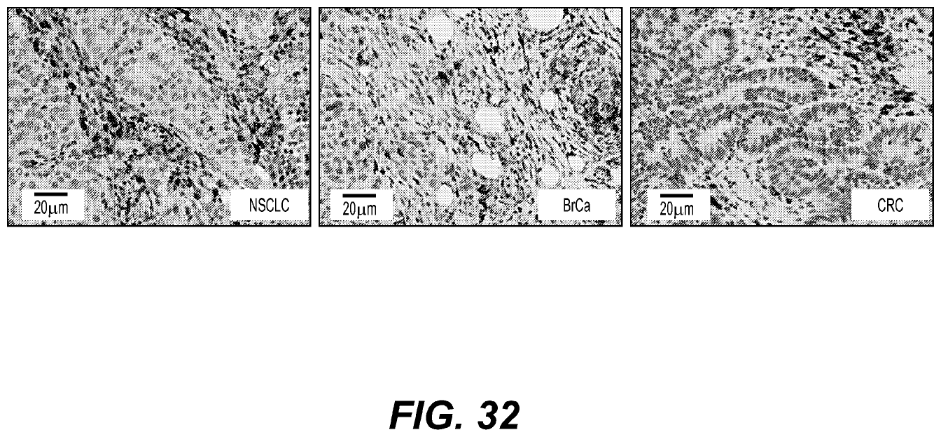

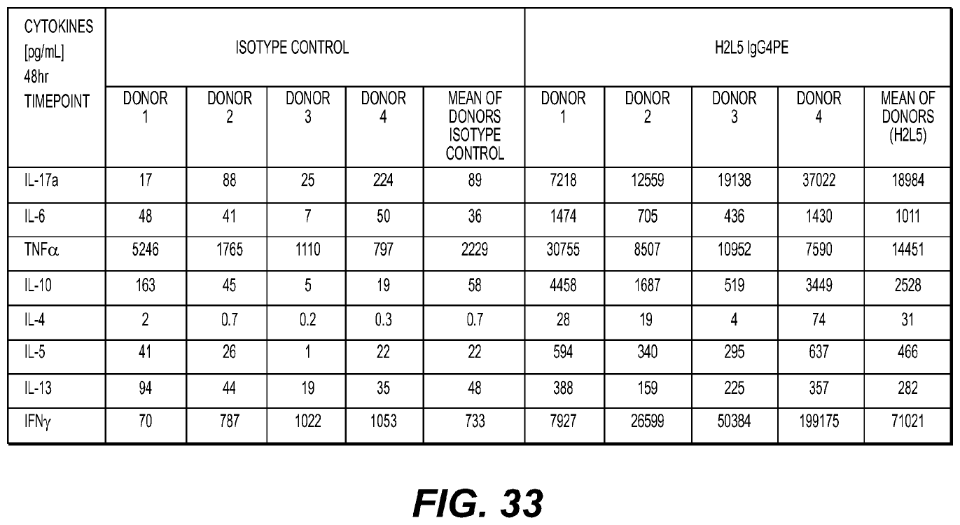

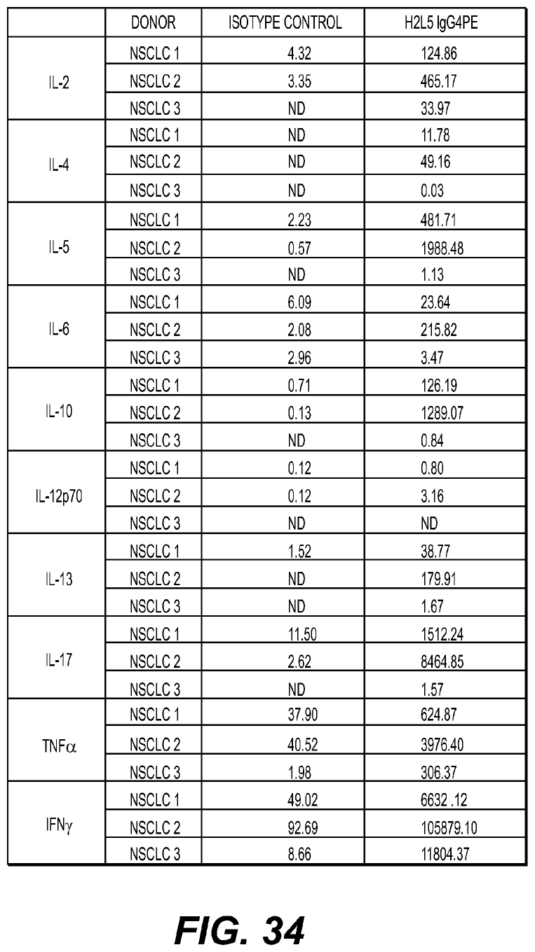

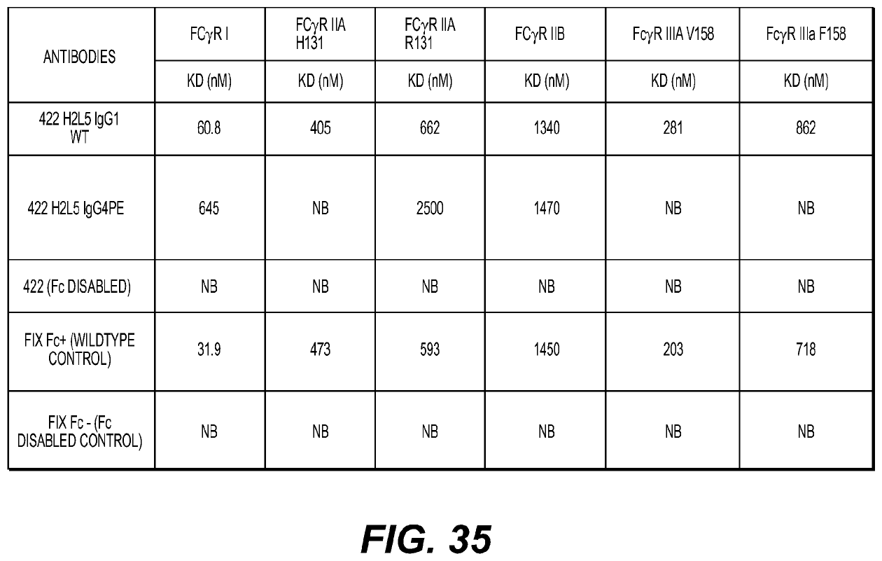

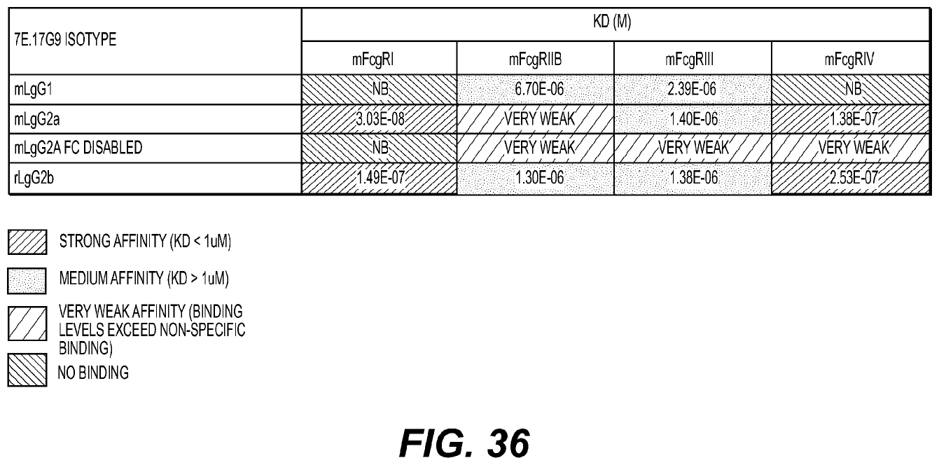

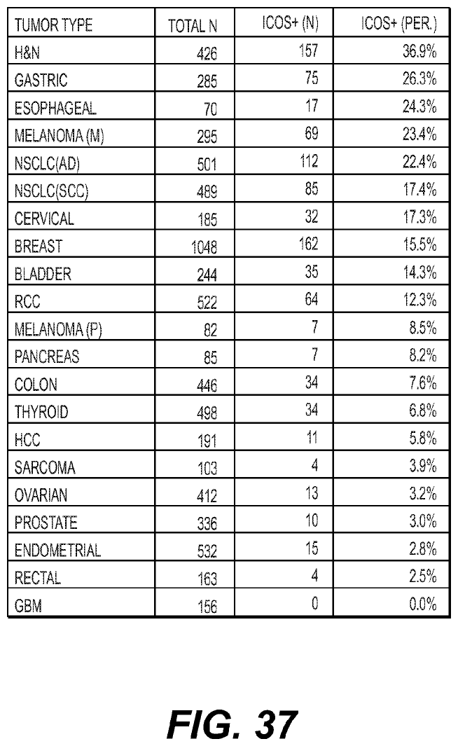

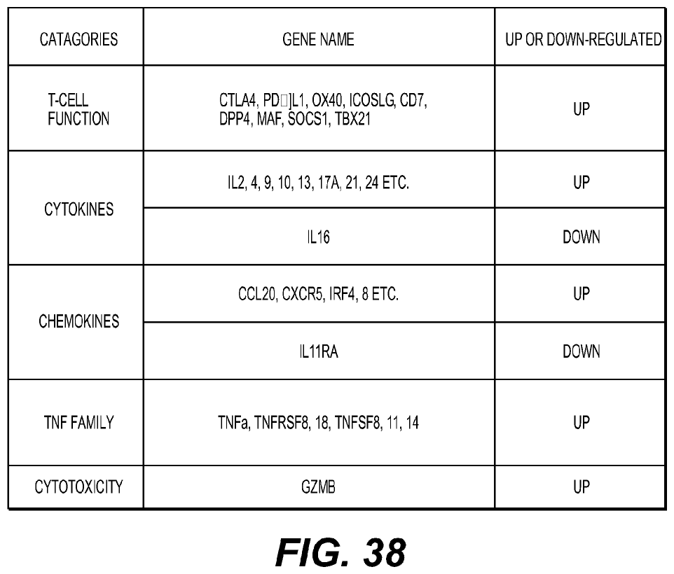

H2L5 bound to human ICOS with an affinity of 1.34 nM (FIG. 15A), which is approximately 17-fold higher than the native ICOS-L/CD275 interaction (FIG. 15B). H2L5 did not bind to murine ICOS or to human CD28 or CTLA-4, the two nearest structurally related protein. This contrasts with the native human ICOS-L, which binds both CTLA-4 and CD28 (Yao, S. et al. Immunity 34(5), 729-40. (2011)). H2L5 blocked ICOS/ICOS-L binding by flow cytometry and competed partially (≤50%) with ICOS-L in binding to ICOS at concentrations above 1 g/mL in MSD immunoassays (FIGS. 22A-22B). H2L5 also bound to both CD4 and CD8 T-cells in activated PBMC samples from healthy human donors (FIG. 15C). ICOS has previously been shown to activate AKT in response to ICOS-L binding in human T cells (Okamoto, N. et al. Biochem Biophys Res Commun. 310(3): 691-702. (2003)); pre-activated primary human CD4 T cells demonstrated an increase in pAKT and pGSK30 in response to treatment with H2L5 (FIG. 15D). H2L5 significantly increased CD4 and CD8 T-cell activation, when used in a plate-bound format together with anti-CD3, as measured by CD69 expression (FIG. 15E) and proliferation (FIG. 15F). Minimal activation was observed with H2L5 in the absence of anti-CD3 treatment, indicating that it does not have superagonist activity under these assay conditions.

The plate-bound H2L5 antibody induced a dose-dependent increase in T_{H}1, T_{H}2 and T_{H}17 cytokines, IFN-γ, TNF-α, IL-17a, IL-10, IL-6 and to a lesser extent IL-2, IL-5 and IL-13 in PBMC from healthy donors (HD) (FIG. 15G, FIG. 23A, FIG. 33). A similar profile of cytokine induction was observed in PBMC from NSCLC patients, with strong induction of IFN-γ, and lower levels of other cytokines including TNF-α, IL-10 and IL-2 (FIG. 15H, FIG. 34). A dose-dependent increase in T-cell activation markers: CD25, OX40 and CD69 on both CD4 and CD8 T cells was also observed with HD following stimulation with plate-bound anti-CD3 and H2L5 (FIG. 23B). With isolated human CD3⁺ T cells, treatment with H2L5 led to a significant increase in the mRNA expression of the T_{H}1 transcription factor T-Bet (FIG. 15I) as well as the cytotoxic molecule Granzyme-B (FIG. 15J). A significant decrease in L-Selectin expression was observed, indicating a transition towards an activated effector phenotype (FIG. 15K). The ability of the plate-bound H2L5 antibody to costimulate T cells isolated ex vivo from disaggregated tumors in the presence of anti-CD3 after 6 days of culture, was also assessed. A concentration dependent and robust increase in IFN-γ was seen in 9/10 donors (FIG. 15L), along with less robust induction of IL-17 and IL-10 compared with healthy PBMC, as well as low-undetectable levels of T_{H}2 cytokines (IL-5 and IL-13) (FIG. 24A-D). Significant increases in activation markers OX40 (FIG. 15M), CD25 (FIG. 15N) and LAG3 (FIG. 25A) were observed on CD8 T cells, in addition to a modest increase in CD8+PD-1+ cells and CD4+CD25+Foxp3+(T_{reg}) cells (FIGS. 25B-25D). Only low percentage of ICOS-L expressing cells were observed with most donors (FIG. 25C)

Altogether, these data show that H2L5 is a potent ICOS agonist, capable of driving T-cell activation and proliferation, but is not a superagonist capable of driving T-cell activation in the absence of TCR stimulation.

### Antibody Isotype and FcγR-Engagement is Critical for H2L5 Function

FcγR-mediated crosslinking is critical for agonist antibody function (Dahal, L. N. et al. Immunol Rev. 268(1): 104-22. (2015); Furness A. J. et al. Trends in Immunology 35(7): 290-298 (2014)). The results described in FIG. 15 utilized plate-bound antibody, which overcomes the need for FcγR cross-linking and suggests that an antibody isotype capable of engaging FcγR and mediating crosslinking is key to achieving optimal ICOS agonism. To formally assess this, we cloned the heavy and light chain variable regions of H2L5 and expressed them as different human IgG isotypes (IgG1, IgG2, IgG4PE and IgG1 Fc-disabled [amino acid (AA) substitutions L235A and G237A) (Bartholomew, M. et al. Immunology 85(1): 41-8 (1995)). The binding of the different H2L5 isotype variants was determined against human FcγRI, FcγRIIa (H131), FcγRIIa (R131), FcγRIIb, FcγRIIIa (V158) and FcγRIIIa (F158) and demonstrated expected patterns of binding (FIG. 35). The IgG4PE contained two AA substitutions from native human IgG4; glutamic acid for leucine at residue 235 (Kabat, E. A., et al. Sequences of Proteins of Immunological Interest, 5th Ed. U.S. Dept. of Health and Human Services, Bethesda, Md., NIH Publication no. 91-3242. (1991)) and substitution of proline for serine at residue 228 (EU numbering) to reduce antigen binding fragment (Fab) arm exchange with native IgG4 (Manjula, P. et al. The Journal of Immunology. 164:1925-1933. (2000), Rispens, T. et al. J. Am. Chem. Soc. 133 (26):10302-10311. (2011)) and decrease binding to activating FcγR and C1q, while retaining binding to the inhibitory FcγRIIb. In PBMC assays the H2L5 IgG1 antibody decreased both CD4 and CD8 T-cell proliferation when added in solution in greater than 50% of donors tested (FIG. 16A). In contrast, IgG2, IgG4PE and Fc-disabled isotype variants of H2L5 did not result in substantial inhibition of either CD4 or CD8 T-cell proliferation in any donors tested, while the H2L5 IgG4PE format resulted in increased proliferation in a subset of the donors (FIG. 16A). We next tested whether the inhibitory effect of H2L5 IgG1 was due to ADCC via NK cells in the PBMC mixture. In PBMCs from 10 healthy donors (HD), the inhibitory effect of H2L5 IgG1 on both CD4 and CD8 populations was lost when NK cells were removed from the PBMC pool (FIG. 16B). The H2L5 isotype variants were also tested in a reporter assay that detects engagement of FcγRIIIa, the primary activating FcγR responsible for NK-mediated ADCC in humans. While the H2L5 IgG1 induced a significant increase in luciferase signaling, when incubated with activated T cells, neither the H2L5 IgG4PE nor H2L5 Fc-disabled antibodies induced FcγRIIIa-mediated signaling (FIG. 26A). Additionally, H2L5 IgG1 induced T-cell death in an NK-dependent manner whilst neither IgG4PE nor Fc-disabled H2L5 resulted in any significant increase in cell death as compared to isotype controls (FIG. 16C).

Previous studies have reported that receptor density may influence the susceptibility of T cells to killing by ADCC, leading to potential preferential depletion of different T-cell subsets, which may differ in the tumor microenvironment compared to the lymphoid tissue (Furness A. J. et al. Trends in Immunology 35(7): 290-298 (2014)). The level of expression of ICOS on CD4, CD8 and T_{reg} freshly isolated from different tumors was determined by flow cytometry. Although there was higher expression observed on T_{reg} vs CD4 and CD8 T cells, this was heterogeneous with some tumors showing overlapping levels between these populations; consequently, ICOS high expression was not a distinct feature of T_{reg} (FIG. 16D). To further evaluate the relative contribution of Fc isotype on potential depletion by ADCC, the CD4, CD8 and T_{reg} cells were purified directly from different cancer patients and the level of ICOS receptor density was correlated with ability of the IgG1 or IgG4PE isotype of H2L5 to stimulate FcγRIIIA in an ex vivo reporter assay (FIG. 16E, FIG. 26B). T cells isolated from tumors did not stimulate FcγRIIIa when incubated with the H2L5 IgG4PE isotype; whereas incubation with H2L5 IgG1 did lead to some (variable) stimulation. In some tumors, FcRγIIIa receptor engagement was seen with T_{reg}, CD4 and CD8, especially at doses of 1-10 μg/mL supporting the idea that selective ADCC deletion of T_{reg} without affecting CD4 and CD8 may not be universally possible in all tumors (e.g. Breast 1001202 patient sample conventional CD4 T cells induced similar stimulation to the T_{reg} at doses of 1-10 μg/ml; FIG. 16F, FIG. 26B).

Based on the above data, the isotype selected for development was the engineered IgG4PE antibody, H2L5.

### H2L5 Induces FcγR-Mediated Agonism of TCR Dependent T-Cell Activation.

H2L5 was tested with isolated human CD4 T cells in both a plate-bound (immobilized antibody) format as well as in solution. H2L5 in the immobilized format, which simulates membrane-bound FcγR-dependent crosslinking, induced significantly greater levels of IFN-γ compared with the soluble antibody (FIG. 17A). The importance of FcγR engagement for optimal H2L5 agonist activity was further confirmed in an activated human PBMC assay, where H2L5 resulted in >2-fold induction of IFN-γ; whereas the Fc-disabled version of H2L5 had no cytokine induction activity compared with the isotype control (FIG. 17B). The IgG4PE and Fc-disabled versions of H2L5 were also tested in a modified mixed lymphocyte reactions (MLR). The H2L5 IgG4PE mAb provided >2-fold induction of IFN-γ whereas the Fc-disabled H2L5 mAb had no activity compared with the isotype control (FIG. 17C). Next, a CD4 T cell/CD14 monocyte donor-matched co-culture assay was utilized to determine whether FcγR-expressing monocytes increased the agonist potential of soluble H2L5. Like the MLR assay format, H2L5 only induced IFN-γ when tested as an IgG4PE isotype; the Fc-disabled antibody showed no significant cytokine induction compared with the isotype control. The addition of monocytes, which are known to express FcγRs including FcγRII isoforms, resulted in a significant increase in H2L5 IgG4PE-induced cytokine production compared with T cells alone. Interaction with FcγRIIB has been shown to be critical for the agonistic activity of other immunomodulatory antibodies targeting TNF-α family receptors as well as CD28 (Bartholomew, M. et al. Immunology 85(1): 41-8 (1995); Bartholomaeus, P. et al. J Immunol. 192(5): 2091-8. (2014)). Conversely, the addition of an FcγR-blocking antibody completely inhibited the H2L5-induced cytokine induction (FIG. 17D). These results indicate that H2L5 can achieve FcγR engagement likely via the FcγRIIB as seen with other IgG4 agonist antibodies (Bartholomaeus, P. et al. J Immunol. 192(5): 2091-8. (2014), Hussain, K. et al. Blood 125 (1): 102-110 (2015)), while avoiding ADCC killing of ICOS+ T cells, as seen with the IgG1 isotype.

To assess its localization and mobilization at the cell surface, H2L5 was fluorescently labeled, added to primary activated human CD3+ T cell cultures, alongside DCs and imaged. Following binding, H2L5 rapidly polarized on the T cell surface. The mobilized T cells began scanning the culture until binding with a dendritic cell (DC) was initiated. In instances where T cells were in cellular contact with DCs, H2L5 accumulated at the point of contact (FIG. 17E). Additional studies using co-cultures of human DC and T-cells demonstrated that H2L5 was rapidly co-localized with CD28 and to a lesser extent CD4 at the polarized caps of activated T cells as well as the subsequent immune synapses that formed upon T-cell binding to DC (FIG. 17F). These results indicate that ICOS induces human T-cell mobilization and is co-located at the immune synapse following T cell activation.

### H2L5 Induces an Effector Memory Phenotype and Antitumor Activity In Vivo

The in vivo functionality of H2L5 was evaluated in a human PBMC engrafted NSG mouse model implanted with A2058 tumors. This model induces a Graft-versus-Host Disease (GVHD) response and has been used previously to study effector and memory T-cell activity (23). In the blood of H2L5-treated mice, the number of human T cells decreased in a dose-dependent manner (FIG. 18A), while a corresponding increase in CD69 expression (representing T-cell activation) was observed (FIG. 18B). The Fc-disabled version of H2L5 showed similar, albeit weaker, trends than H2L5 IgG4PE suggesting that the disappearance of cells was not due to ADCC. H2L5 induced a dose-dependent increase in CD4+CD45R0+CD62L-effector memory (TEM) cells (FIG. 18C), and CD8⁺CD45RO⁻CD62L⁻ terminally differentiated CD8 effector cells (TEMRA) (FIG. 18D). H2L5 was next tested in human PBMC engrafted NSG mice harboring either HCT116 or A549 tumors. Detection of H2L5 binding to ICOS+ T cells (CD4, CD8 and T_{reg}), by a human anti-IgG4 fluorescent labelled antibody, was observed in blood and tumor at doses of 0.4 and to lesser extent 0.04 mg/kg demonstrating target engagement in mice bearing the A549 tumors (FIG. 18E, FIGS. 27A-27B). Mice treated with anti-PD-1 IgG4 antibody (Keytruda) also showed the detection of bound antibody using the same detection reagent. Treatment of mice with H2L5 was associated with an increase in the CD8:T_{reg} ratio in the A549 tumors, comparable to that seen in mice treated with anti-PD-1. (FIG. 18F). H2L5 resulted in significant tumor growth inhibition in both HCT116 and A549 tumor models (FIGS. 18G-18H). In the A549 model where the GVHD response was less severe, tumor growth inhibition resulted in dose-dependent increase in survival beyond 50 days (FIG. 181). These experiments suggest that doses of 0.4 mg/kg, which correlate with successful engagement of the ICOS receptor, result in subsequent pharmacological effects associated with T-cell activation in blood and tumor and reduction of tumor growth.

### The Fc Isotype of Murine Anti-ICOS Antibody Influences Efficacy in Syngeneic Tumors

Studies in the literature using CTLA-4, PD-L1, OX40 and CD40 have shown that selection of the Fc isotype of mAbs can significantly influence efficacy in different tumor models (Dahal, L. N. et al. Immunol Rev. 268(1): 104-22. (2015); Furness A. J. et al. Trends in Immunology 35(7): 290-298 (2014)). To generate a surrogate mouse anti-ICOS antibody equivalent to a human IgG4 in terms of FcγR binding, (FIG. 36) the anti-mouse ICOS antibody 7E-17G9 was cloned into murine (m) IgG1 and mIgG2a isotypes and tested in 2 different tumor models. The 7E-17G9 antibody showed agonistic activity in plate-bound format with anti-CD3 (FIG. 28). In the EMT6 model the mIgG1 antibody showed greater efficacy than the mIgG2a especially at higher doses (>5 mg/kg, 100 μg/mouse) with both survival (FIG. 19A) and tumor growth inhibition (FIG. 29A). However, both isotypes showed only modest dose-dependent efficacy as monotherapy in the CT26 model (FIG. 19B, FIG. 29B). As described above for the human IgG1, the mIgG2a depleting antibody may be less effective than the mIgG1, since it has the potential to deplete both T_{eff} and T_{reg}. A significantly higher CD8:T_{reg} ratio (FIG. 19C) was observed for EMT6 vs CT26, prior to treatment (100 mm³) and both EMT6 and CT26 models showed an increase in the percentage of ICOS positive CD4 and CD8 and T_{reg} cells in tumor vs spleen (FIG. 19D, FIG. 30) but higher percentage of ICOS CD8 positive cells were observed in spleen in EMT6 vs CT26 (80% vs 10%). Although high levels of ICOS expression on T_{reg} from tumor-infiltrating lymphocytes (TILs) were observed for both EMT6 and CT26 tumors, ICOS levels on CD8 TILs were approximately 10-fold higher in EMT6 than CT26 (30,000 vs 3000 MFI). This suggests that high ICOS expression on CD8 in both periphery and tumor may be associated with response with the agonistic activity of the mIgG1 antibody in the EMT6 model (FIGS. 19E-19G). To further explore mechanisms of the agonist anti-ICOS mAb in mice bearing EMT6 breast tumors, effects on TCR diversity were investigated; significant changes in the number of unique circulating TCR clones and a corresponding increase in TCR clonality in the blood of ICOS mAb treated mice was noted (FIGS. 31A-31C). Most clones that expanded in mouse blood in response to ICOS agonist mAb treatment were also found in tumors (FIG. 19H). These findings indicate that a small pool of tumor-reactive T-cell clones expand in response to ICOS mAb treatment.

### Characterization of the ICOS/ICOS-L Pathway in Human Cancers

To further explore the translation of an ICOS agonist mAb as an anti-tumor therapeutic antibody, human solid tumors from the TCGA database were ranked by ICOS mRNA expression (FIG. 26). Highest expression was observed in head and neck, gastric, esophageal, melanoma, NSCLC, cervical and breast cancer. Expression was confirmed in NSCLC by singleplex IHC (FIG. 32). As the H2L5 agonist mAb mode of action is designed to phenocopy ICOS-L activity, the co-expression of mRNA for ICOS and ICOS-L was analyzed in these tumor types (FIG. 20A). ICOS expression was often not co-expressed with ICOS-L, supporting the hypothesis that H2L5 may augment the low level ICOS signaling in these tumors. We also assessed the relative expression of PD-L1 in the same samples. Expression of PD-L1 has been associated with increased T-cell infiltration and used as a predictive biomarker to enrich for patients responding to anti-PD-1/PD-L1 treatment in different indications. Overall there was a clear association between PD-L1 and ICOS expression but this was variable between different indications (FIG. 20A). These results were confirmed by IHC staining for expression of CD4, CD8 and FOXP3 and tended to localize with ICOS in immune infiltrates in NSCLC (FIG. 20B).

The presence of key cell types in the tumor microenvironment was analyzed by flow cytometry in biopsies from different tumor types. Of the CD45+ leukocyte population, CD3 T cells appeared to be the dominant cell type, ranging from 20-80%; other cell types such as B cells, macrophages, monocytes, NK cells and DC were also present (FIG. 20C). These cells types express FcγR including FcγRIIb, which may provide the cross-linking required for agonistic activity of H2L5 in the tumor microenvironment (Furness A. J. et al. Trends in Immunology 35(7): 290-298 (2014)). The composition of the T-cell sub-populations averaged CD4 (68%), CD8 (30%) and T_{reg} (2%) although there was considerable heterogeneity between different tumors. When tumor types were analyzed separately, the heterogeneity between CD8 and T_{reg} was clear with NSCLC and RCC showing a high CD8/T_{reg} ratio (FIG. 20D). Further analysis by multiplex IHC was performed to characterize ICOS expression of different T-cell sub-types. Co-expression of ICOS was observed on a proportion of CD3+PD-1+ cells, especially in head and neck, esophageal, NSCLC and melanoma supporting a rationale for combination treatment with anti-PD-1 therapies (FIGS. 20E-20F).

Next, the effects of H2L5 costimulation on gene expression by purified human T cells was determined using the Human PanCancer-Immune profiling panel to identify an ICOS gene signature. Compared with anti-CD3 alone, 120 genes were differentially induced with 85 upregulated and 35 down-regulated (FIG. 20G). Several immune related genes or pathways were induced by H2L5 compared to anti-CD3 alone including T_{H}1 cytokines, and chemokines, T-cell function and cytotoxicity, and TNF family members (FIG. 38). The top genes identified from EMT6 mouse tumors treated with 7E.17G9 that overlapped with the human ICOS-induced signature are shown in FIG. 20H. This information is being used to guide development of an ICOS transcriptional signature to monitor pharmacodynamic effects of H2L5 in early clinical studies.

ICOS Agonist Treatment Induces PD-1/PD-L1 in Tumors and Demonstrates Increased Activity in Combination with Anti-PD-1 Blockade

PD-L1, a known IFN-γ responsive gene, as well as PD-1, increased significantly in the tumors of ICOS mAb treated mice (FIGS. 21A-21B). Human PBMCs were collected from six cancer patients and treated with H2L5, which resulted in a significant increase in PD-1 expression on both CD4 and CD8 T cells (FIG. 21C). In addition, NSCLC and melanoma patients treated with anti-PD-1 therapies showed an increase in ICOS expression on CD4 T cells in peripheral blood compared with pre-treatment (FIG. 21D). Therefore, we tested whether combination with a PD-1 blocking antibody could augment the antitumor activity of the ICOS agonist mAb. The ICOS agonist mAb (7E17G9 mIgG1 isotype) was dosed alone or in combination with anti-PD-1 antibody in mice with established EMT6 tumors (150 mm³). Combination resulted in an increased antitumor response and long-term survival (90% of mice) as compared with monotherapy treatment with ICOS or PD-1 antibodies alone (FIG. 21E). The combination of H2L5 and anti-PD-1 (pembrolizumab) was also assessed in the humanized mouse model and resulted in enhanced antitumor response to A549 tumors compared with monotherapy alone (FIG. 21F) These data show that the addition of an ICOS agonist antibody significantly improved the antitumor activity induced by a PD-1 antibody.

H2L5 was further tested alone or in combination with pembrolizumab in primary resected tumors from 6 patients with NSCLC in an ex vivo assay. While treatment with H2L5 alone resulted in a significant increase in IFN-γ in 4/6 of the NSCLC tumor samples tested, the combination of H2L5 and pembrolizumab resulted in a significant increase in IFN-γ as compared to pembrolizumab alone and an increase in 5/6 samples as compared to H2L5 treatment alone (FIG. 21G). The H2L5 combination with pembrolizumab was also tested in a modified allogeneic human MLR assay where combination treatment resulted in increased IFN-γ levels as compared to either agent alone in 3/3 different healthy donor pairs (FIG. 21H).

### DISCUSSION

We have presented the first full characterization of the immunological activity and therapeutic potential of a first-in-class, humanized IgG4 anti-ICOS agonist mAb, H2L5. We have demonstrated that the H2L5 IgG4PE agonist antibody induces significant activation and clonal expansion of both CD4 and CD8 T cells in vitro and in vivo. These T cells have increased effector function through increased expression of T_{H}1 cytokines such as IFN-γ, as well as increased production of cytotoxic factors such as Granzyme B. ICOS antibody-activated T cells displayed increased tissue-homing to tumors with significant accumulation and infiltration resulting in antitumor responses. Prior reports using ICOS^{-/-} and ICOS-L^{-/-} mice as well as blocking antibodies to ICOS-L have demonstrated the importance of ICOS for the expansion, survival and function of both CD4 and CD8 TEM cells in mice (4, 24-25). Additionally, patients with common variable immune deficiency, which is characterized by a homozygous loss of ICOS, have been found to have fewer memory T cells, specifically those which are CD62L^{low} (26). Our studies using a novel human ICOS-specific agonist antibody have confirmed the role of ICOS for inducing this population of memory T cells, providing a viable therapeutic approach for targeting this important mechanism in humans.

We show that the engineered form of IgG4 that incorporates the mutations S228P and L235E (EU numbering) relative to the native human IgG4 is the preferred antibody isotype over IgG1 for achieving agonist function against human ICOS. These AA changes prevent heterogeneous exchange with native IgG4 (Rispens, T. et al. J. Am. Chem. Soc. 133 (26): 10302-10311. (2011)). The IgG4PE isotype also has reduced binding to activating FcγR and C1q compared to human IgG1, thereby diminishing the cytotoxic potential of H2L5 that could result in depletion of ICOS-positive T cells through antibody-dependent or complement-dependent mechanisms, respectively (Manjula, P. et al. The Journal of Immunology. 164:1925-1933. (2000)). Our in vitro studies have shown that the IgG1 isotype of H2L5 (the initial isotype of H2L5 planned for development) is able to kill activated CD4 and CD8 T cells expressing high levels of ICOS, as well as reduce their proliferation in an NK-dependent manner; this was not seen not seen with the IgG4PE isotype. Importantly, the IgG4PE isotype retains functional binding to FcγRIIb (the inhibitory FcγR), critical for enabling agonist activity against several stimulatory immune receptors (Bartholomaeus, P. et al. J Immunol. 192(5): 2091-8. (2014); Hussain, K. et al. Blood 125 (1): 102-110 (2015), Aalberse, R. C and Schuurman, J. Immunology 105(1): 9-19. (2002); Schuurman J. and Parren P. W. Curr Opin Immunol. (2016); White A. L. et al. J Immunol. 187(4):1754-63. (2011); White A. L. et al. J Immunol 193 (4): 1828-1835 (2014); Dahal R. et al. Cancer Cell. 29(6):820-31. (2016); Yu X. et al. Cancer Cell 33 (4): 664-675 (2018)), which may also be essential for ICOS agonist activity and associated antitumor effects in humans. The selection of the IgG4PE isotype was further supported by in vivo studies using the anti-murine ICOS 7E17G9 surrogate antibody, where the murine IgG1 isotype showed greater efficacy than the deleting IgG2a antibody in the EMT6 syngeneic model. Murine IgG1 has a similar profile to human IgG4, with low binding to activating FcγR receptors, yet retaining binding to some Fcγ-receptors including, inhibitory FcγRIIB and inducing Fc-dependent crosslinking to improve agonism of the anti-ICOS antibody; whereas the murine IgG2a can bind the activating FcγR, like human IgG1, and is able to mediate effective deletion. Studies performed with CTLA-4, PD-L1, OX40 and CD40 have shown that selection of the Fc isotype of mAbs can significantly influence efficacy in different tumor models; however, this needs to be optimised for each target, depending on relative expression levels on different cell types (e.g. CD8 vs T_{eff} vs T_{reg}) and mode of action of the antibody (agonism/deletion) and epitope specificity (Dahal, L. N. et al. Immunol Rev. 268(1): 104-22. (2015), Furness A. J. et al., Trends in Immunology 35(7): 290-298 (2014), Yu X. et al. Cancer Cell 33 (4): 664-675 (2018)). Ex vivo human tumors contain varying proportions of B cells, macrophages and DCs, known to express FcγRIIB, which is critical to mediate the FcγR crosslinking required for H2L5 in the tumor microenvironment (Furness A. J. et al. Trends in Immunology 35(7): 290-298 (2014), Hussain, K. et al. Blood 125 (1): 102-110 (2015), Aalberse, R. C and Schuurman, J. Immunology 105(1): 9-19. (2002); Schuurman J. and Parren P. W. Curr Opin Immunol. (2016); White A. L. et al. J Immunol. 187(4):1754-63. (2011); White A. L. et al. J Immunol 193 (4): 1828-1835 (2014); Dahal R. et al. Cancer Cell. 29(6):820-31. (2016); Yu X. et al. Cancer Cell 33 (4): 664-675 (2018)). A balance in favour of inhibitory FcγRIIB vs activating FcγR is often seen in the immunosuppressive environment of human tumors, which may favor the cross-linking of H2L5 and enhance its agonist activity (Furness A. J. et al. Trends in Immunology 35(7): 290-298 (2014), Dahal, L. et al. Cancer Research 77 (13) 3619-3631 (2017)).

Given the agonist activity of H2L5 described above, one factor which must be considered, is the expression of ICOS on T_{reg} cells in the tumor microenvironment. The relationship of ICOS positive T-cell subsets on response to the murine 7E. 17E7 IgG1 surrogate antibody in the EMT-6 and CT26 murine tumor models was explored. A higher ratio of ICOS+CD8:T_{reg} was observed at baseline in the EMT6 model vs CT26 in tumors, which may be one factor leading to greater efficacy in EMT6 model observed with 7E.17G9 IgG1. Similarly, in the humanized mouse model tumor reduction by H2L5 was associated with an increased CD8:T_{reg} ratio. In this model, the response to treatment with H2L5 monotherapy was similar to anti-PD-1 treatment. These results suggest that the presence of ICOS-positive T_{reg} does not preclude the ability of an ICOS agonist to provide therapeutic benefit.

Human tumors express varying proportions of CD4 and CD8 T_{eff} and T_{reg}, with considerable variability between tumor types. The percentage of ICOS positive cells and the level of ICOS expression was found to be heterogeneous between different cell types, with a trend for higher ICOS levels on T_{reg} although in many patients there was overlap between expression of ICOS on T_{reg} vs CD4 and CD8 T cells. The IgG1 isotype but not the IgG4 isotype of H2L5 anti-ICOS antibody was able to bind and induce activation of the FcγRIIIA luciferase reporter assay with of ex vivo purified T_{reg}, and to some extent CD8 and CD4 cells. While the IgG1 mediated activity in this assay system was found to correlate with ICOS receptor density, as has been reported with other targets such as CTLA-4, OX40 and GITR (19), the differential expression of ICOS on T_{reg} vs T_{eff} was less prominent. Furthermore, since ICOS expression on T_{eff} is enhanced with anti-PD-1 and anti-CTLA-4 treatment, this suggests that there may not be a large therapeutic window for the IgG1 isotypes to mediate selective depletion of T_{reg} over T_{eff} in vivo. Based on the data described above, strategies for the development of H2L5 include selection of tumor types which have a high CD8:T_{reg} ratio and high ICOS expression on CD8 T cells, (e.g., NSCLC) and development of rational combinations with agents that decrease the abundance of, or limit the function of, T_{reg}.

A rational combination partner supported by our data is a PD-1/PD-L1 blocking antibody. ICOS agonist antibody treatment significantly induced PD-1 on human T cells as well as PD-1 and PD-L1 expression in tumors of treated mice; furthermore, anti-PD-1 treatment was also shown to induce expression of ICOS on CD4 and CD8 T_{eff} cells. Like the combinatorial activity observed in mice, the human ICOS agonist H2L5 in combination with the PD-1 blocking antibody, pembrolizumab, demonstrated increased cytokine production relative to either agent alone in ex vivo human immune cell assays. This robust induction of IFN-γ by H2L5 IgG4PE supports the rationale of anti-PD-1 combination as IFN-γ is known to act on negative feedback by up-regulation of PD-L1 (Mandal, M. et al. Clinical cancer Research 22(10):2329-2334).

Single-agent treatment with anti-PD-1 or anti-PD-L1 antibodies has demonstrated response rates between 15-30% across many solid tumors (e.g. bladder, head and neck, lung) (Hoos, A. Nat. Rev. Drug Disc. 15(4):235-47. (2016)). Emerging clinical data using PD-1 or PD-L1 antibodies in combination with other agents has shown signals of increased activity in some settings, however with substantial added toxicity in some instances (Larkin, J. et al. N Engl J Med. 373:23-34. (2015); Forde P. M., et al. New England Journal of Medicine (2018); Xu, X. et al. Int. J Cancer. 142: 2344-2354 (2018)). Several predictive biomarkers have shown mechanism of response and resistance to anti-PD-1 treatment and support rationale for combinations (Gibney G. T. et al. Lancet Oncology 17(12): 542-551(2016); Tumeh P. C. et al. Nature. 515 (7528): 568-71. (2014); Taube, T. M. et al. Clin. Cancer Research. 20 (19):5064-5074 (2014)). It has been shown the tumor mutational burden, is a correlate of the generation of neoantigens which stimulate expansion of the endogenous tumor specific repertoire and is associated with response to anti-PD-1 therapy (Schumacher, T. N. and Schreiber, R. D. Science 348: 69-74 (2015)). The clonality and degree of T-cell infiltration in tumors has recently been shown to be an important positive predictor for immunotherapy outcomes in cancer (Xu, X. et al. Int. J Cancer. 142: 2344-2354 (2018)). Our findings demonstrate that with the murine surrogate antibody, TCR clones were expanded and shared between both blood and tumor. Furthermore, H2L5, which induces T-cell proliferation, expansion, and tumor infiltration, may be complimentary to other immunotherapy agents with distinct mechanisms of action.

Results described in Example 2 were obtained using the following materials and methods:

### Materials and Methods

### Humanized H2L5 Antibody

H2L5 is a humanized variant of the murine mAb clone 422.2 obtained from the lab of Daniel Olive, Institut Paoli-Calmettes, INSERM (Marseille, France). The murine antibody was generated using standard hybridoma technology by immunizing BALB/c mice intraperitoneally with recombinant human ICOS-Fc using COS7 cells.

### Cell Lines and Primary Cell Cultures

Murine tumor cell lines EMT6 (ATCC # CRL-2755) and CT26 (ATCC # CRL-2638) and human cell lines A549 (ATCC # CCL-185) A2058 (ATCC # CRL-11147), HCT116 (ATCC # CCL-247) were expanded and frozen upon receipt and used at low passage (≤10 passages) for inoculation to mice. Prior to in vivo use, cell lines were tested by PCR and confirmed negative for pathogens including mycoplasma using the mouse/rat comprehensive CLEAR panel (Charles River Research Animal Diagnostic Services).

All patient material was obtained with the appropriate informed written consent in accordance with the GSK human biological sample management (HBSM) policy and SOP. Whole blood in sodium heparin tubes (BD Biosciences) and surgically resected tumor tissues from cancer patients were obtained from Avaden Biosciences (Seattle) shipped overnight by post. Primary T cells or PBMC from healthy human donors were purified from whole blood collected in sodium heparin tubes at the GSK on-site blood donation units with appropriate consent and in accordance with the GSK HBSM policy. PBMC were isolated by density gradient centrifugation through Histopaque. T-cells were isolated by negative selection using Dynabeads™ Untouched™ Human T-cell kit (Life Technologies) or RosetteSep human CD4 or CD8 T-cell enrichment kits (StemCell) for binding and functional assays. Isolated T cells were pre-activated with plate-bound anti-CD3 (clone OKT3, eBioscience) and anti-CD28 (clone CD28.2, eBioscience) for 48-96 hrs to upregulate ICOS expression.

### Mice, Tumor Challenge and Treatment

All studies were conducted in accordance with the GSK Policy on the Care, Welfare and Treatment of Laboratory Animals and were reviewed by the Institutional Animal Care and Use Committee either at GSK or by the ethical review process at the institution where the work was performed. 6-8 week old female BALB/c mice (Harlan/Envigo) were utilized for in vivo studies in a fully accredited AAALAC facility. 5×10⁴ cells/mouse CT26 mouse colon carcinoma or 1×10⁵ EMT6 mouse mammary carcinoma tumor cells were inoculated subcutaneously into the right flank. Prior to initiation of treatment, mice (n=10/treatment group) were randomized with the Study Director software package (Studylog Systems) when the tumors reached 100 mm³ unless otherwise specified. ANOVA was used to ensure similarity between groups (P>0.9).

The study investigator was blinded during the group allocation and assessed the final outcome to ensure that group distributions were acceptable for study initiation (P>0.9).

Based on inter-individual variability in tumor growth rates from 5 separate studies in the CT26 syngeneic model, 10 mice per group were justified as the optimal number necessary to observe an effect size of approximately 0.8 between control and drug-treated groups and to generate statistically significant data.

Tumor bearing mice received the mouse anti-ICOS (clone 7E.17G9) in different isotype backgrounds or H2L5 and/or mouse anti-PD-1 (clone RMP1-14) or an isotype control in saline via intraperitoneal injection twice weekly starting on randomization day for a total of 6 doses. Tumor measurement of greater than 2,000 mm³ for an individual mouse and/or development of open ulcerations resulted in mice being removed from study.

### Binding Studies

The affinity and kinetics of H2L5 binding to rabbit Fc-tagged recombinant extracellular human or cynomolgus ICOS (generated in-house) was determined using a Biacore™ T200 (GE Healthcare™). The ICOS binding data was fitted to a 1:1 kinetics model using the T200 data analysis software. Cell surface binding of H2L5 to both freshly isolated unactivated and CD3/CD28 activated CD4 and CD8 T cells was determined via detection of anti-human IgG, kappa light chain FITC (Sigma) binding to H2L5 by flow cytometry.

### Antibodies

The following anti-human antibodies were used for flow cytometry analysis, CD4 (RPA-T4, BD Biosciences), CD8 (RPA-T8, Biolegend), CD69 (FN50, Biolegend), OX40 (ACT-35, eBioscience), Ki67 (B56, BD Biosciences), ICOS (ISA3, eBioscience). The following anti-mouse antibodies were used for flow cytometry analysis: CD3 (145-2C11, BD Biosciences), CD4 (RM4-5, BD Biosciences), CD8 (53-6.7, BD Biosciences), CD25 (PC61, BD Biosciences), CD44 (IM7, Biolegend), CD62L (MEL14, BD Biosciences), FOXP3 (Fjk-16s, eBioscience), ICOS (C398.4a, Biolegend), Ki67 (16A8, Biolegend). Apoptotis was measured using the Annexin V kit with 7-AAD (Biolegend). For flow cytometry analysis of the human PBMC mouse model the following antibodies were used: CD45 (HI30, BD Biosciences), CD3 (UCHT1, Biolegend), CD4 (SK3, BD Biosciences), CD45RO (UCHL1, Biolegend), CD62L (SK11, BD Biosciences). p-AKT (S473, #4060 and T308, #13038), total Akt (#9272), pGSK3-α (#5558), total GSK3-α (#12456), pS6 (S235/236, #2211 and S240/244, #5364), total S6 (#2317), and pERK (#9101) (all from Cell Signaling Technology) were used for Western Blots.

### ADCC Assays

Whole PBMC or NK depleted PBMC were activated with plate-bound anti-CD3 and anti-CD28 antibodies. Cells were incubated with anti-ICOS antibodies (H2L5 IgG1, H2L5 IgG4PE and H2L5 Fc-disabled) or control antibodies at 10 μg/mL final concentration for 24 hours. Cells were stained with anti-CD8 and CD4 antibodies followed by incubation with NIR Live/Dead dye (Invitrogen). Stained cells were analyzed by flow cytometry (FACSCanto, BD Biosciences) to measure T-cell killing based on NIR Live/Dead cell dye staining.

In the FcγRIIIa engagement reporter bioassay (Promega), anti-CD3/CD28 pre-activated CD4 T cells were incubated with the anti-ICOS and control antibodies for 45 minutes prior to the addition of Jurkat-FcγRIIIA-NFAT-luciferase effector cells at an E:T cell ratio of 6:1. ONE-GLO luciferase reagent was added to each well after 6 hrs of treatment and luminescence intensity measured to determine engagement between the target T cells and the effector cells on a Victor plate reader (Perkin Elmer). CD4, CD8 and T_{reg} populations were purified from either donor PBMC pre-activated with anti-CD3/CD28 or disaggregated tumor cells and tested directly ex vivo at 6:1 E:T ratio in presence of IgG1 or IgG4PE H2L5 antibodies.

### Functional Assays

H2L5 was tested in human PBMC assays either in a plate-bound format with concurrent CD3 stimulation using freshly isolated PBMC or in a soluble format in CD3/CD28 pre-stimulated PBMC as described earlier. For PBMC from cancer patients, an overnight rest step was included prior to treatment initiation. 10 μg/mL soluble pembrolizumab was used in in vitro assays to study effects of combination. Cytokine concentrations in supernatants from these assays were measured using bespoke human multiplex meso-scale detection (MSD) kits (Meso Scale Diagnostics).

Human monocytes were isolated from whole blood of healthy human donors, using CD14 MicroBeads (Miltenyi Biotec) for the T cell:monocyte mixed culture assays. T cell and monocytes were donor matched. CD3/CD28 pre-stimulated T cells and monocytes were mixed at 2:1 ratio in AIM-V serum-free media and cultured together with anti-CD3 dynabeads (Life Technologies), 100 IU of recombinant human IL-2 and 100 ng/ml of M-CSF (Peprotech) prior to incubating with soluble H2L5 or other control antibodies at 37° C. for 4 days. 20 μg/mL human Fc block (B564220) (BD biosciences) or anti-CD32 mAb (MCA1075EL, Clone AT10) (AbD serotec) were used to test the role of FcγR cross linking.

For the MLR assays, monocytes (Lonza, Switzerland) were grown in GM-CSF and IL-4 (Pepro Tech) supplemented LGM-3 media (Lonza) for 9 days for differentiating into mDCs and TNFα (R&D Systems) for an additional day before use in the MLR assay. The mDC-T cell (1:10 ratio) mix was treated with 10 μg/mL soluble H2L5 Fc-disabled or the isotype control antibodies either in the presence of anti-CD3 beads at a 1:10 bead to cell ratio (Life Technologies) or CEFT peptide mix (0.02 μg/mL) (JPT Peptide Technologies) for 4 days before collecting the supernatants for cytokine analysis by MSD.

Primary patient tumors were dissociated using GentleMACS (Miltenyi Biotec) tissue dissociator. TIL were expanded in IL-2 supplemented RPMI media (Baldan et al., 2015) before treating with anti-CD3 plus H2L5. Alternatively, tumor dissociated cells were directly cultured ex vivo for up to 6 days following stimulation with anti-CD3 plus H2L5 with 100 ng/ml IL-2 added after 24 hours.

For PBMC assays testing different H2L5 isotypes, anonymized leukocyte cones from healthy donors were obtained from the National Blood Service at Southampton General Hospital, UK and used within 4 hours. Use of human samples was approved by local ethical committees in accordance with the Declaration of Helsinki. PBMC were isolated by density gradient centrifugation (Lymphoprep) and cultured in RPMI medium 1640 (Life Technologies) supplemented with glutamine (2 mM), sodium pyruvate (1 mM), penicillin, and streptomycin (100 IU/mL) at 37° C. in 5% CO₂.

Proliferation assays were performed as detailed previously (35). Briefly, fresh PBMC were labelled with 1 μM carboxyfluorescein succinimidyl ester (CFSE) and cultured at high density (1×10⁷/mL) for 48 hours prior to antibody stimulations. For the PBMC stimulation, cells were transferred into round-bottomed 96-well plates at 1×10⁵ per well and stimulated with 1 μg/ml OKT3 (plate-bound) and 5 μg/ml (soluble) H2L5 mAbs. On day 6, cells were labelled with anti-CD8-e450 (SK-1, eBioscience) and anti-CD4-APC (RPA-T4, Insight Biotechnology), and proliferation assessed by CFSE dilution on a FACSCantoII flow cytometer (BD Biosciences). Results are expressed as % divided cells compared to the unstimulated cells. NK depletion was performed using CD56 micro beads (Miltenyi Biotec) according to the manufacturer's instructions post 48 hours high density culture (Hussain et al. Blood 2014).

### Immunofluorescence Studies

Unstimulated and CD3/CD28 stimulated T cells were Fc blocked with 20 μg/mL human Fc block (B564220) (BD biosciences) or anti-CD32 mAb (MCA1075EL, Clone AT10) (AbD serotec) to test the role of FcγR cross linking and then treated with 6 μg/mL cold labeled antibody (anti-ICOS or IgG4PE isotype control) on ice for 1 hr. Cells were washed in cold buffer and transferred to 37° C. for various times (0, 5, 15, 30 minutes and 1 hour) to allow protein trafficking before fixing with freshly prepared 4% paraformaldehyde (Sigma). Samples 1 or 2 hours after the initial pulse at 37° C. were re-pulsed with Alexa Fluor 647 labeled anti-ICOS for 30 minutes at 37° C., washed and fixed in paraformaldehyde. The cells were transferred to Poly-L-lysine coated coverslips for 15 minutes and then mounted on slides in ProLong Gold with DAPI (Invitrogen). Analysis of the samples was performed using a ZEISS LSM510 Meta Confocal microscope with a 63× oil immersion lens.

### Human T-Cell Gene Expression

Whole blood was obtained from healthy volunteer donors (n=6) at the GSK on-site Blood Donation Unit and T cells were purified using RosetteSep™ Human T-Cell Enrichment Cocktail (Stemcell Technologies) as described above. The cells were re-suspended (5×10⁶ cells/mL) in AIM-V culture media (Gibco) and incubated in 96-well plates (Falcon) that were sequentially pre-coated with 0.6 μg/mL of mouse anti-human CD3 mAb (eBioscience) and 10 μg/mL of anti-human ICOS or corresponding isotype control mAbs—mouse IgG2 α κ (eBioscience) and IgG4PE. After 24 hours of incubation at 37° C. and 5% CO₂, cells were pelleted, suspended in RLT buffer (Qiagen), and stored at −80° C. for RNA isolation. Total RNA was extracted using the RNeasy Mini QIAcube Kit (Qiagen). RNA expression levels were determined by NanoString nCounter Analysis System. 50 ng of RNA was used in each reaction for gene signature using NanoString Human PanCancer Immune profiling CodeSet according to the manufacturer's instructions. Raw data was normalized using built-in positive controls and house-keeping genes (nCounter Expression Data Analysis Guide, NanoString). ArrayStudio (OmicSoft) and GraphPad Prism (GraphPad Software) were used for further analysis and graphs.

### ICOS/ICOS-L Competition Assay

MSD plates were incubated overnight at 4° C. with 10 μg/mL recombinant ICOS protein (R&D Systems) diluted in PBS. Plates were washed and blocked before adding isotype control or H2L5 in a 7-point dose curve. After overnight incubation and washes, the plates were incubated with 1 μg/mL human ICOS ligand (B7-H2) (R&D Systems) followed by incubation with 10 μg/mL biotinylated anti-human ICOS ligand (B7-H2) (R&D Systems) antibody. Sulfo-tagged streptavidin at 10 μg/mL in Diluent 100 was used for detection of the biotinylated ligand. The plates were read immediately following MSD Read buffer addition on a MSD MESO Quick Plex SQ 120 and data analyzed on MSD workbench software. Flow cytometry was also used to investigate competition between cell surface ICOS expressed by anti-CD3/CD28 activated T cells and ICOS-L by H2L5. Activated T cells were incubated with different concentrations of recombinant ICOS-L and then incubated with H2L5 and MFI of ICOS CD4+ and CD8+ ICOS cells determined.

### Human PBMC Mouse Model

Adult immunodeficient NOD/SCID/IL-2Rγnull (NSG) mice (Jackson Labs) were injected with human PBMC (20×10⁶ per mouse) by intravenous injection via the tail vein. Mice were implanted with human tumor cell lines A2058, A549, HCT116 (1×10⁶) 1-3 days post human PBMC injection; mice were administrated isotype control or anti-human ICOS antibodies at doses ranging from 0.004 mg/kg to 1.2 mg/kg by intraperitoneal injection twice weekly for 3 weeks. Tumor bearing mice received the mouse anti-ICOS (clone 7E. 17G9) in different isotype backgrounds or H2L5 and/or Pembrolizumab (Merck; NDC #0006-3026-02) antibodies or isotype controls in saline via intraperitoneal injection twice weekly starting on randomization day for a total of 6 doses. Tumor measurement of greater than 2,000 mm³ for an individual mouse and/or development of open ulcerations resulted in mice being removed from study.

Spleens and whole blood were collected post-euthanization at 24 hrs post 2^{nd} or 4^{th} dose of antibodies. Splenocytes were isolated by mechanical dissociation followed by RBC lysis with LCK lysis buffer (Lonza) and antibody staining whereas whole blood was stained with the appropriate antibodies before RBC lysis with FACSlyse (BD Biosciences). All samples were evaluated by flow cytometry on FACScanto (BD) as described below.

### Western Blotting

Activated T cells were treated with H2L5 or an isotype control for up to 48 hours. CD4+ T cells were prestimulated with CD3/CD28 Dynabeads® (ThermoFisher) at a cell-to-bead ratio of 1:20 for 48 hours, allowed to rest in the absence of stimulation for 24 hours, and then treated with isotype control antibody or H2L5 (10 μg/mL) in the presence of plate-bound anti-CD3 antibody. Cells were lysed with cell lysis buffer (Cell Signaling Technologies) containing protease and phosphatase inhibitors (Roche). 25-30 μg of protein was run on 4-12% Bis-Tris gels (Invitrogen) and transferred onto nitrocellulose membranes (Invitrogen). Membranes were blocked using LI-COR Odyssey Blocking Buffer and subsequently immunoblotted using the primary and secondary antibodies and scanned on a LI-COR Odyssey imaging system.

### FACS Analysis

Non-specific binding on activated T-cells was blocked by incubation with human or mouse Fc block (Miltenyi Biotec) as appropriate prior to the incubation with detection antibodies to cell surface markers conjugated to different fluorophores on ice for 30 minutes. For intracellular staining, the cells were fixed and permeabilized using the Transcription Factor Buffer set (BD biosciences). After compensation, data were acquired on FACS Canto II or Fortessa (BD biosciences) and analyzed with FACSDiva (BD) or Flowjo (Treestar) software.

### Immunohistochemistry

Immunohistochemical detection of ICOS in non-small cell lung cancer (NSCLC), breast cancer (BrCA) TNBrCa, and colorectal cancer (CRC), was performed using a rabbit anti-human CD278 mAb (clone SP98; Spring Biosciences) on a Leica Bond RX with associated platform reagents. DAB (3, 3′-diaminobenzidine) was used for target detection. Sections were counter stained with Hematoxylin (All scale bars=20 μm).

Clarient MultiOmyx platform (Neogenomics, California), a multiplexed immunofluorescence (IF) assay was used to evaluate expression of ICOS, PD-1, CD3, CD4 and CD8 among other T-cell markers on FFPE tumor tissues obtained from vendors vetted by GSK HBS group as described above. The iterative process included a round of staining with a Cy3 and Cy5 conjugated antibody each and imaging, followed by dye inactivation, background fluorescence imaging and subtraction of the background before the repeating this cycle for all markers in the panel.

### Statistical Analysis

One-way ANOVA or Student's t-tests were used as specified in the figure legend. Data were analyzed with GraphPad Prism software (GraphPad) and p values of <0.05 were considered statistically significant. (*P≤0.05; **P≤0.01; ***P≤0.005; ****P≤0.0001).

1. A method of treating cancer in a patient in need thereof, the method comprising administering to the patient an effective amount of an agent directed to human ICOS and an effective amount of an agent directed to human PD1 or human PD-L1 sequentially, wherein administration of the agent directed to human ICOS is followed by administration of the agent directed to human PD1 or human PD-L1.

2. The method of claim 1, wherein the agent directed to human ICOS is an anti-ICOS antibody or antigen binding portion thereof.

3. The method of claim 2, wherein the anti-ICOS antibody is an ICOS agonist.

4. The method of claim 2, wherein the anti-ICOS antibody comprises a V_{H} domain comprising an amino acid sequence at least 90% identical to the amino acid sequence set forth in SEQ ID NO:7; and a V_{L} domain comprising an amino acid sequence at least 90% identical to the amino acid sequence as set forth in SEQ ID NO:8.

5. The method of claim 2, wherein the anti-ICOS antibody comprises a V_{H} domain comprising the amino acid sequence set forth in SEQ ID NO:7 and a V_{L} domain comprising the amino acid sequence as set forth in SEQ ID NO:8.

6. The method of claim 1, wherein the agent directed to human PD1 is an anti-PD1 antibody or antigen binding portion thereof.

7. The method of claim 6, wherein the anti-PD1 antibody is a PD1 antagonist.

8. The method of claim 6, wherein the anti-PD1 antibody is pembrolizumab.

9. The method of claim 6, wherein the anti-PD1 antibody is nivolumab.

10. The method of claim 1, wherein the agent directed to human PD-L1 is an anti-PD-L1 antibody or antigen binding portion thereof.

11. The method of claim 10, wherein the anti-PD-L1 antibody is a PD1 antagonist.

12. The method of claim 1, wherein the agent directed to human ICOS or anti-ICOS antibody or antigen binding portion thereof is administered once every week, once every two weeks, once every three weeks, or once every four weeks.

13. The method of claim 1, wherein the agent directed to human PD1 or human PD-L1 or anti-PD1 antibody or antigen binding portion thereof or anti-PD-L1 antibody or antigen binding portion thereof is administered once every week, once every two weeks, once every three weeks, or once every four weeks.

14. The method of claim 1, wherein the cancer is selected from the group consisting of colorectal cancer (CRC), gastric, esophageal, cervical, bladder, breast, head and neck, ovarian, melanoma, renal cell carcinoma (RCC), EC squamous cell, non-small cell lung carcinoma, mesothelioma, pancreatic, and prostate cancer.

15. The method of claim 1, wherein the agent directed to human ICOS, or anti-ICOS antibody or antigen binding portion thereof, is administered as an intravenous (IV) infusion.

16. The method of claim 1, wherein the agent directed to human PD1 or human PDL1, or anti-PD1 antibody or antigen binding portion thereof or anti-PDL1 antibody or antigen binding portion thereof, is administered as an intravenous (IV) infusion.

17. The method of claim 1, wherein the start of administration of the agent directed to human PD1 or human PDL1, or anti-PD1 antibody or antigen binding portion thereof or anti-PDL1 antibody or antigen binding portion thereof, is initiated at a time point selected from 1 week, 2 weeks, 3 weeks, and 4 weeks after the start of the administration of the agent directed to human ICOS, or anti-ICOS antibody or antigen binding portion thereof.

18. The method of claim 1, wherein the agent directed to human ICOS, or the anti-ICOS antibody or antigen binding portion thereof, and the agent directed to human PD1 or human PDL1, or the anti-PD1 antibody or antigen binding portion thereof or the anti-PDL1 antibody or antigen binding portion thereof, are administered to said human until said human shows disease progression or unacceptable toxicity.

19.-40. (canceled)

41. A method of treating cancer comprising administering an anti-ICOS antibody or antigen binding portion thereof and an anti-PD1 antibody or antigen binding portion thereof, wherein the anti-ICOS antibody or antigen binding portion thereof and an anti-PD1 antibody or antigen binding portion thereof are sequentially administered, and wherein administration of the anti-ICOS antibody or antigen binding portion thereof is followed by administration of the anti-PD1 antibody or antigen binding portion thereof.

42. A method of treating cancer comprising administering an anti-ICOS antibody or antigen binding portion thereof and an anti-PDL1 antibody or antigen binding portion thereof, wherein the anti-ICOS antibody or antigen binding portion thereof and an anti-PDL1 antibody or antigen binding portion thereof are sequentially administered, and wherein administration of the anti-ICOS antibody or antigen binding portion thereof is followed by administration of the anti-PDL1 antibody or antigen binding portion thereof.
